SEQUENCE LISTING (188 sequences)

SEQ ID NO: 1 - protein, 163 aa, Chlamydomonas reinhardtii
MNTKYNKEFLLYLAGFVDGDGSIIAQIKPNQSYKFKHQLSLAFQVTQKTQRRWFLDKLVDEIGVGYVRDRGSVSDYILSEIKPLHNFLTQLQPFLKLKQKQANLVLKIIWRLPSAKESPDKFLEVCTWVDQIAALNDSKTRKTTSETVRAVLDSLSEKKKSSP

SEQ ID NO: 2 - DNA, 22 bp, Homo sapiens
ttctctgtggtgagaaaattta

SEQ ID NO: 3 - DNA, 22 bp, Homo sapiens
ttcactattttgaaatatacag

SEQ ID NO: 4 - DNA, 22 bp, Homo sapiens
tattttgaaatatacagcacaa

SEQ ID NO: 5 - DNA, 22 bp, Homo sapiens
taactttgttcatattactatg

SEQ ID NO: 6 - DNA, 22 bp, Homo sapiens
actttgttcatattactatgca

SEQ ID NO: 7 - DNA, 22 bp, Homo sapiens
catattactatgcaatagaaca

SEQ ID NO: 8 - DNA, 22 bp, Homo sapiens
cactagaacttattactccttt

SEQ ID NO: 9 - DNA, 22 bp, Homo sapiens
tttcagttgatgaacaggcagt

SEQ ID NO: 10 - DNA, 22 bp, Homo sapiens
agttttggatcaagaataatat

SEQ ID NO: 11 - DNA, 22 bp, Homo sapiens
aaaaatattttgaaagggaata

SEQ ID NO: 12 - DNA, 22 bp, Homo sapiens
ccaaataatttattacaatgtt

SEQ ID NO: 13 - DNA, 22 bp, Homo sapiens
atctttcttttaatcaataaat

SEQ ID NO: 14 - DNA, 22 bp, Homo sapiens
ttttaatcaataaatatattca

SEQ ID NO: 15 - DNA, 22 bp, Homo sapiens
accttccatttaaaatcagctt

SEQ ID NO: 16 - DNA, 22 bp, Homo sapiens
tcagcttttatattgagtattt

SEQ ID NO: 17 - DNA, 22 bp, Homo sapiens
gcttttatattgagtatttttt

SEQ ID NO: 18 - DNA, 22 bp, Homo sapiens
taaaatgttgtgtgtacatgct

SEQ ID NO: 19 - DNA, 22 bp, Homo sapiens
atgttgtgtgtacatgctaggt

SEQ ID NO: 20 - DNA, 22 bp, Homo sapiens
gctaggtgtgtatattaatttt

SEQ ID NO: 21 - DNA, 22 bp, Homo sapiens
atttgttacttgaaactaaact

SEQ ID NO: 22 - DNA, 22 bp, Homo sapiens
ctaaactctgcaaatgcaggaa

SEQ ID NO: 23 - DNA, 22 bp, Homo sapiens
gtgatatctttgtcagtataac

SEQ ID NO: 24 - DNA, 22 bp, Homo sapiens
aaaaaatatacgctatatctct

SEQ ID NO: 25 - DNA, 22 bp, Homo sapiens
atctgttttacataatccatct

SEQ ID NO: 26 - DNA, 22 bp, Homo sapiens
ctgttttacataatccatctat

SEQ ID NO: 27 - DNA, 22 bp, Homo sapiens
ctatttttcttgatccatatgc

SEQ ID NO: 28 - DNA, 22 bp, Homo sapiens
catatgcttttacctgcaggcg

SEQ ID NO: 29 - DNA, 22 bp, Homo sapiens
aaattacttttgactgttgttg

SEQ ID NO: 30 - DNA, 22 bp, Homo sapiens
tgactgttgttgtcatcattat

SEQ ID NO: 31 - DNA, 22 bp, Homo sapiens
ttgttgtcatcattatattact

SEQ ID NO: 32 - DNA, 22 bp, Homo sapiens
ttgtcatcattatattactaga

SEQ ID NO: 33 - DNA, 22 bp, Homo sapiens
atcattatattactagaaagaa

SEQ ID NO: 34 - DNA, 22 bp, Homo sapiens
aaaattatcataatgataatat

SEQ ID NO: 35 - DNA, 22 bp, Homo sapiens
atggactttttgtgtcaggatg

SEQ ID NO: 36 - DNA, 22 bp, Homo sapiens
ggactttttgtgtcaggatgag

SEQ ID NO: 37 - DNA, 22 bp, Homo sapiens
ggagctggtttatctgataaac

SEQ ID NO: 38 - DNA, 22 bp, Homo sapiens
attgaatctgtgacagagggaa

SEQ ID NO: 39 - DNA, 22 bp, Homo sapiens
agggaagcatcgtaacagcaag

SEQ ID NO: 40 - DNA, 22 bp, Homo sapiens
gggcagtgtgtatttcggcttt

SEQ ID NO: 41 - DNA, 22 bp, Homo sapiens
tatattctattgacaaaatgcc

SEQ ID NO: 42 - DNA, 22 bp, Homo sapiens
taattgttggtacttattgaca

SEQ ID NO: 43 - DNA, 22 bp, Homo sapiens
tgttggtacttattgacatttt

SEQ ID NO: 44 - DNA, 22 bp, Homo sapiens
ttttatggtttatgttaatagg

SEQ ID NO: 45 - DNA, 22 bp, Homo sapiens
agtttttttttaatactgtgac

SEQ ID NO: 46 - DNA, 22 bp, Homo sapiens
tttaatactgtgactaacctat

SEQ ID NO: 47 - DNA, 22 bp, Homo sapiens
tttcacctctcgtatccacgat

SEQ ID NO: 48 - DNA, 22 bp, Homo sapiens
tcacctctcgtatccacgatca

SEQ ID NO: 49 - DNA, 22 bp, Homo sapiens
ctcgtatccacgatcactaaga

SEQ ID NO: 50 - DNA, 22 bp, Homo sapiens
ccaaatactttgttcatgttta

SEQ ID NO: 51 - DNA, 22 bp, Homo sapiens
ggaacatccttgtggggacaag

SEQ ID NO: 52 - DNA, 22 bp, Homo sapiens
aatttgctcttgaaaaggtttc

SEQ ID NO: 53 - DNA, 22 bp, Homo sapiens
ctaattgatttgtaggacatta

SEQ ID NO: 54 - DNA, 22 bp, Homo sapiens
ttccctgacacataaaaggtgt

SEQ ID NO: 55 - DNA, 22 bp, Homo sapiens
ccctgacacataaaaggtgtct

SEQ ID NO: 56 - DNA, 22 bp, Homo sapiens
ctttctgtcttgtatcctttgg

SEQ ID NO: 57 - DNA, 22 bp, Homo sapiens
atcctttggatatgggcatgtc

SEQ ID NO: 58 - DNA, 22 bp, Homo sapiens
tggatatgggcatgtcagtttc

SEQ ID NO: 59 - DNA, 22 bp, Homo sapiens
gatatgggcatgtcagtttcat

SEQ ID NO: 60 - DNA, 22 bp, Homo sapiens
gaaattttcacatggagctttt

SEQ ID NO: 61 - DNA, 22 bp, Homo sapiens
tttctttctttgccagtacaac

SEQ ID NO: 62 - DNA, 22 bp, Homo sapiens
tctttgccagtacaactgcatg

SEQ ID NO: 63 - DNA, 22 bp, Homo sapiens
tttggtatcttacaggaactcc

SEQ ID NO: 64 - DNA, 22 bp, Homo sapiens
aagaatatttcatgagagatta

SEQ ID NO: 65 - DNA, 22 bp, Homo sapiens
gaatatttcatgagagattata

SEQ ID NO: 66 - DNA, 22 bp, Homo sapiens
tgagagattataagcagggtga

SEQ ID NO: 67 - DNA, 22 bp, Homo sapiens
aaggcactaacattaaagaacc

SEQ ID NO: 68 - DNA, 22 bp, Homo sapiens
tcaacagcagtaaagaaatttt

SEQ ID NO: 69 - DNA, 22 bp, Homo sapiens
ttctttttttcatatactaaaa

SEQ ID NO: 70 - DNA, 22 bp, Homo sapiens
ctaaaatatatacttgtggcta

SEQ ID NO: 71 - DNA, 22 bp, Homo sapiens
tgaatatcttcaatatatttta

SEQ ID NO: 72 - DNA, 22 bp, Homo sapiens
caattataaatgattgttttgt

SEQ ID NO: 73 - DNA, 22 bp, Homo sapiens
atgattgttttgtaggaaagac

SEQ ID NO: 74 - DNA, 22 bp, Homo sapiens
tcatattttgtacaaaataaac

SEQ ID NO: 75 - DNA, 22 bp, Homo sapiens
atacgtgtattgcttgtactac

SEQ ID NO: 76 - DNA, 22 bp, Homo sapiens
gtattgcttgtactactcactg

SEQ ID NO: 77 - DNA, 22 bp, Homo sapiens
actgaatctacacaactgccct

SEQ ID NO: 78 - DNA, 22 bp, Homo sapiens
tgaatctacacaactgccctta

SEQ ID NO: 79 - DNA, 22 bp, Homo sapiens
caactgcccttatgacatttac

SEQ ID NO: 80 - DNA, 22 bp, Homo sapiens
ggtaaatacatgaaaaatgctt

SEQ ID NO: 81 - DNA, 22 bp, Homo sapiens
ttgccttgcttactgcttattg

SEQ ID NO: 82 - DNA, 22 bp, Homo sapiens
gcttactgcttattgctagtac

SEQ ID NO: 83 - DNA, 22 bp, Homo sapiens
tagtactgaacaaatgttagaa

SEQ ID NO: 84 - DNA, 22 bp, Homo sapiens
actgaacaaatgttagaactga

SEQ ID NO: 85 - DNA, 22 bp, Homo sapiens
aagatttatttaatgactttga

SEQ ID NO: 86 - DNA, 22 bp, Homo sapiens
cagtatttcatgtctaaataga

SEQ ID NO: 87 - DNA, 22 bp, Homo sapiens
ggtttttcttcactgctggcca

SEQ ID NO: 88 - DNA, 22 bp, Homo sapiens
caatctgaaataaaaagaaaaa

SEQ ID NO: 89 - DNA, 22 bp, Homo sapiens
ctgctcccagtataaaatacag

SEQ ID NO: 90 - DNA, 22 bp, Homo sapiens
aagaacgtttcattggctttga

SEQ ID NO: 91 - DNA, 22 bp, Homo sapiens
acttcctattcaagggaatttt

SEQ ID NO: 92 - DNA, 22 bp, Homo sapiens
tgttttttcttgaataaaaaaa

SEQ ID NO: 93 - DNA, 22 bp, Homo sapiens
ttttcttgaataaaaaaaaaat

SEQ ID NO: 94 - DNA, 22 bp, Homo sapiens
ttgttttctttaccacttccac

SEQ ID NO: 95 - DNA, 22 bp, Homo sapiens
acaatgtatatgattgttactg

SEQ ID NO: 96 - DNA, 22 bp, Homo sapiens
tgtatatgattgttactgagaa

SEQ ID NO: 97 - DNA, 22 bp, Homo sapiens
cttgtccaggcatgagaatgag

SEQ ID NO: 98 - DNA, 22 bp, Homo sapiens
tgtccaggcatgagaatgagca

SEQ ID NO: 99 - DNA, 22 bp, Homo sapiens
aatcgttttttaaaaaattgtt

SEQ ID NO: 100 - DNA, 22 bp, Homo sapiens
ttctaccatgtattgctaaaca

SEQ ID NO: 101 - DNA, 22 bp, Homo sapiens
taccatgtattgctaaacaaag

SEQ ID NO: 102 - DNA, 22 bp, Homo sapiens
tataatgtcatgaataagagtt

SEQ ID NO: 103 - DNA, 22 bp, Homo sapiens
atgtcatgaataagagtttggc

SEQ ID NO: 104 - DNA, 22 bp, Homo sapiens
ttttcctttttgcaaaaaccca

SEQ ID NO: 105 - DNA, 22 bp, Homo sapiens
ttcctttttgcaaaaacccaaa

SEQ ID NO: 106 - DNA, 22 bp, Homo sapiens
agttcttaggcaactgtttctc

SEQ ID NO: 107 - DNA, 22 bp, Homo sapiens
tctctctcagcaaacacattac

SEQ ID NO: 108 - DNA, 22 bp, Homo sapiens
taagtataatcaaggatataaa

SEQ ID NO: 109 - DNA, 22 bp, Homo sapiens
agtagccatacattaaaaagga

SEQ ID NO: 110 - DNA, 22 bp, Homo sapiens
aggaaatatacaaaaaaaaaaa

SEQ ID NO: 111 - DNA, 22 bp, Homo sapiens
agaaaccttacaagaatagttg

SEQ ID NO: 112 - DNA, 22 bp, Homo sapiens
caagaatagttgtctcagttaa

SEQ ID NO: 113 - DNA, 22 bp, Homo sapiens
atctattttataccaaataagt

SEQ ID NO: 114 - DNA, 22 bp, Homo sapiens
ttataccaaataagtcactcaa

SEQ ID NO: 115 - DNA, 22 bp, Homo sapiens
tttgttttggcactacgcagcc

SEQ ID NO: 116 - DNA, 22 bp, Homo sapiens
taaggataattgaaagagagct

SEQ ID NO: 117 - DNA, 22 bp, Homo sapiens
agaaaagtaacaaaacataaga

SEQ ID NO: 118 - DNA, 22 bp, Homo sapiens
ttaaagttggcatttatgcaat

SEQ ID NO: 119 - DNA, 22 bp, Homo sapiens
agttggcatttatgcaatgcca

SEQ ID NO: 120 - DNA, 22 bp, Homo sapiens
aacatgtttttaatacaaatag

SEQ ID NO: 121 - DNA, 22 bp, Homo sapiens
tacattgatgtaaatatggttt

SEQ ID NO: 122 - DNA, 22 bp, Homo sapiens
atatcttttatatttgtgaatg

SEQ ID NO: 123 - DNA, 22 bp, Homo sapiens
cttttatatttgtgaatgatta

SEQ ID NO: 124 - DNA, 22 bp, Homo sapiens
tgtgaatgattaagaaaaataa

SEQ ID NO: 125 - DNA, 22 bp, Homo sapiens
aattgttatacattaaagtttt

SEQ ID NO: 126 - DNA, 22 bp, Homo sapiens
aaagttttttcacttgtaacag

SEQ ID NO: 127 - DNA, 22 bp, Homo sapiens
taacagctttcaagcctttcta

SEQ ID NO: 128 - DNA, 22 bp, Homo sapiens
ggtatttaggtattaaagtact

SEQ ID NO: 129 - DNA, 22 bp, Homo sapiens
tactaccttttgaaaaaacaag

SEQ ID NO: 130 - DNA, 22 bp, Homo sapiens
ggaatttctttgtaaaataaac

SEQ ID NO: 131 - DNA, 22 bp, Homo sapiens
aacctgcatttaaaggccttga

SEQ ID NO: 132 - DNA, 22 bp, Homo sapiens
tgagcttgaatacagaagacct

SEQ ID NO: 133 - DNA, 22 bp, Homo sapiens
tgattgtggtcaagccatctct

SEQ ID NO: 134 - DNA, 22 bp, Homo sapiens
ctattctgagtacagagcatac

SEQ ID NO: 135 - protein, 364 aa, Artificial Sequence
  Description of Artificial Sequence Synthetic polypeptide
MAPKKKRKVHMNTKYNKEFLLYLAGFVDGDGSIYAWISPSQTCKFKHRLMLRFIVSQKTQRRWFLDKLVDEIGVGYVQDCGSVSEYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYACILPTQRQKFKHGLTLYFRVTQKTQRRWFLDKLVDEIGVGYVLDFGSVSCYSLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 136 - protein, 364 aa, Artificial Sequence
  Description of Artificial Sequence Synthetic polypeptide
MAPKKKRKVHMNTKYNKEFLLYLAGFVDGDGSIFASIRPRQTSKFKHALALFFVVGQKTQRRWFLDKLVDEIGVGYVYDRGSVSVYQLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIIACIRPHQAYKFKHQLCLSFCVYQKTQRRWFLDKLVDEIGVGYVTDAGSVSSYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 137 - protein, 364 aa, Artificial Sequence
  Description of Artificial Sequence Synthetic polypeptide
MAPKKKRKVHMNTKYNKEFLLYLAGFVDGDGSIFACIQPDQRAKFKHTLRLSFEVGQKTQRRWFLDKLVDEIGVGYVNDSGSVSKYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYATIQPTQCAKFKHQLTLRFSVSQKTQRRWFLDKLVDEIGVGYVCDKGSVSEYMLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 138 - protein, 364 aa, Artificial Sequence
  Description of Artificial Sequence Synthetic polypeptide
MAPKKKRKVHMNTKYNKEFLLYLAGFVDGDGSIYACILPVQRCKFKHGLSLRFMVSQKTQRRWFLDKLVDEIGVGYVYDCGSVSEYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIFASIVPDQRSKFKHGLALRFNVVQKTQRRWFLDKLVDEIGVGYVYDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 139 - DNA, 1791 bp, Artificial Sequence
  Description of Artificial Sequence Synthetic polynucleotide
cacaggtgtccactcccagttcaattacagctcttaaggctagagtacttaatacgactcactataggctagcctcgagccgccaccatggcaccgaagaagaagcgcaaggtgcatatgaatacaaaatataataaagagttcttactctacttagcagggtttgtagacggtgacggttccatctatgcctggatcagtccttcgcaaacgtgtaagttcaagcacaggctgatgctccggttcattgtctcgcagaagacacagcgccgttggttcctcgacaagctggtggacgagatcggtgtgggttacgtgcaggactgtggcagcgtctccgagtaccggctgtccgagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacgcgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctatgcctgtatccttccgactcagcgtcagaagttcaagcacgggctgacgctctatttccgggtcactcagaagacacagcgccgttggttcctcgacaagctggtggacgagatcggtgtgggttacgtgctggactttggcagcgtctcctgttactctctgtcccagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccctaaacagtctctccgagaagaagaagtcgtccccctagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataagatcttgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcg

SEQ ID NO: 140 - DNA, 1791 bp, Artificial Sequence
  Description of Artificial Sequence Synthetic polynucleotide
cacaggtgtccactcccagttcaattacagctcttaaggctagagtacttaatacgactcactataggctagcctcgagccgccaccatggcaccgaagaagaagcgcaaggtgcatatgaatacaaaatataataaagagttcttactctacttagcagggtttgtagacggtgacggttccatctttgcctctatccggcctcggcaaacgagtaagttcaagcacgcgctggctctctttttcgtggtcgggcagaagacacagcgccgttggttcctcgacaagctggtggacgagatcggtgtgggttacgtgtatgaccgtggcagcgtctccgtgtaccagctgtcccagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacgcgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatcattgcctgtatccggcctcatcaagcttataagttcaagcaccagctgtgtctctctttctgtgtctatcagaagacacagcgccgttggttcctcgacaagctggtggacgagatcggtgtgggttacgtgacggacgctggcagcgtctcctcttaccggctgtccgagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccctaaacagtctctccgagaagaagaagtcgtccccctagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataagatcttgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcg

SEQ ID NO: 141 - DNA, 31 bp, Artificial Sequence
  Description of Artificial Sequence Synthetic primer
gaaagaaaatgccaatagtccaaaatagttg

SEQ ID NO: 142 - DNA, 25 bp, Artificial Sequence
  Description of Artificial Sequence Synthetic primer
catattcaaaggacaccacaagttg

SEQ ID NO: 143 - DNA, 1791 bp, Artificial Sequence
  Description of Artificial Sequence Synthetic polynucleotide
cacaggtgtccactcccagttcaattacagctcttaaggctagagtacttaatacgactcactataggctagcctcgagccgccaccatggcaccgaagaagaagcgcaaggtgcatatgaatacaaaatataataaagagttcttactctacttagcagggtttgtagacggtgacggttccatctttgcctgtatccagcctgatcaaagggcgaagttcaagcacacgctgcggctctctttcgaggtcgggcagaagacacagcgccgttggttcctcgacaagctggtggacgagatcggtgtgggttacgtgaatgactctggcagcgtctccaagtacaggctgtcccagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacgcgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctatgccactatccagcctactcaatgtgcgaagttcaagcaccagctgactctccgtttctcggtctctcagaagacacagcgccgttggttcctcgacaagctggtggacgagatcggtgtgggttacgtgtgtgacaagggcagcgtctccgagtacatgctgtccgagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccctaaacagtctctccgagaagaagaagtcgtccccctagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataagatcttgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcg

SEQ ID NO: 144 - DNA, 1791 bp, Artificial Sequence
  Description of Artificial Sequence Synthetic polynucleotide
cacaggtgtccactcccagttcaattacagctcttaaggctagagtacttaatacgactcactataggctagcctcgagccgccaccatggcaccgaagaagaagcgcaaggtgcatatgaatacaaaatataataaagagttcttactctacttagcagggtttgtagacggtgacggttccatctatgcctgtatcttgccggtgcagcgttgtaagttcaagcacgggctgtctctccgattcatggtcagtcagaagacacagcgccgttggttcctcgacaagctggtggacgagatcggtgtgggttacgtgtatgactgtggcagcgtctccgagtacaggctgtccgagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacgcgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctttgcctctatcgtgccggatcagcgtagtaagttcaagcacggtctggctctcaggttcaatgtcgttcagaagacacagcgccgttggttcctcgacaagctggtggacgagatcggtgtgggttacgtgtatgaccagggcagcgtctccgagtacaggctgtccgagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccctaaacagtctctccgagaagaagaagtcgtccccctagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataagatcttgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcg

SEQ ID NO: 145 - DNA, 23 bp, Artificial Sequence
  Description of Artificial Sequence Synthetic primer
ctaaccgagagggtgcttttttc

SEQ ID NO: 146 - DNA, 30 bp, Artificial Sequence
  Description of Artificial Sequence Synthetic primer
gtgtttaggtcaactaatgtgtttattttg

SEQ ID NO: 147 - protein, 364 aa, Artificial Sequence
  Description of Artificial Sequence Synthetic polypeptide
MAPKKKRKVHMNTKYNKEFLLYLAGFVDGDGSIFACIHPSQAYKFKHRLTLHFTVTQKTQRRWFLDKLVDEIGVGYVQDVGSVSQYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSISATIAPAQYGKFKHYLGLRFYVSQKTQRRWFLDKLVDEIGVGYVSDQGSVSRYCLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 148 - protein, 364 aa, Artificial Sequence
  Description of Artificial Sequence Synthetic polypeptide
MAPKKKRKVHMNTKYNKEFLLYLAGFVDGDGSIYACIRPTQSVKFKHDLLLCFDVSQKTQRRWFLDKLVDEIGVGYVYDRGSVSSYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIEPRQQSKFKHQLRLGFSVYQKTQRRWFLDKLVDEIGVGYVRDTGSVSCYCLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 149 - DNA, 22 bp, Artificial Sequence
  Description of Artificial Sequence Synthetic oligonucleotide
ttctgtgatgtgaggacatata

SEQ ID NO: 150 - DNA, 22 bp, Artificial Sequence
  Description of Artificial Sequence Synthetic oligonucleotide
actaatgaaacaccactccaca

SEQ ID NO: 151 - DNA, 26 bp, Artificial Sequence
  Description of Artificial Sequence Synthetic primer
gtcttatcagtcaagagatcatattg

SEQ ID NO: 152 - DNA, 27 bp, Artificial Sequence
  Description of Artificial Sequence Synthetic primer
gtgtcagtaatctctatccctttcatg

SEQ ID NO: 153 - DNA, 214 bp, Mus sp.
agaatttaaatattaacaaactataacactatgattaaatgcttgatattgagtagttattttaatagcctaagtctggaaattaaatactagtaagagaaacttctagaatttaaatattaacaaactataacactatgattaaatgcttgatattgagtagttattttaatagcctaagtctggaaattaaatactagtaagagaaacttct

SEQ ID NO: 154 - DNA, 80 bp, Mus sp.
tttaatagcctaagtctggaaatactccacaggtgatttcagccactttatgaactgctggaagcaaaaatgagatcttt

SEQ ID NO: 155 - DNA, 181 bp, Mus sp.
ttagttagaatttaaatattaacaaactataacactatgattaaatgcttgatattgagtagttattttaatagcctaagtctggaaattaaatactagtaagagaaacttctgtgatgtgaccactccacaggtgatttcagccactttatgaactgctggaagcaaaaatgagatcttt

SEQ ID NO: 156 - DNA, 691 bp, Mus sp.
tataacactatgattaaatgcttgatattgagtagttattttatgtgtcataccttcttggattgtctgtataaatgaattgattttttttcaccaactccaagtatacttaacattttaacataataatttaaaatatccttattccattatgttcattttttaagttgtagatatgatttagctcacagcatacatatatacacatgtattacatatgcatatattatatatatggcagacatatgttttcactaccatatttcacttttgaattatgaatatatgtttaatttctgccatatttccttccctacattgacttctattaatttagtatttcagtagttctaacacattaataataacctagactcaatacagtaatctaacaattatatttgtgcctgtaattctaagttagttaaattcataggttgtgtttctcatagttggccatttgtgaaatataataatatccgaaaagaaagttcaaaaatgtcatgacttcatatagagttattgaaacagtgcccttactttcattctggccatgctagtgacttgatcattcttgtattttacagctaaaacactaccaaaagtgtcaaatccatgatctacatgtttgaccactccacaggtgatttcagccactttatgaactgctggaagcaaaaatgagatcttt

SEQ ID NO: 157 - DNA, 112 bp, Mus sp.
ttgagtagttattttaatagcctaagtctggaaattaaatactagtaagagaaacttctgtgatgtgcacaggtgatttcagccactttatgaactgctggaagcaaaaatg

SEQ ID NO: 158 - DNA, 90 bp, Mus sp.
gatattgagtagttattttaatagcctaagtctggaaattaaatactagtagatttcagccactttatgaactgctggaagcaaaaatga

SEQ ID NO: 159 - DNA, 182 bp, Mus sp.
aatactagtaagagaaacttctgtgatgtgaggactccacaggtgatttcagccactttatgaactgctggaagcaaaaatgagatctttgcaacatgaagcagttgctcagttcattaaactgtgttcaatatttcagccataacatacattagagaatgatttatattgttcaaacattt

SEQ ID NO: 160 - DNA, 172 bp, Mus sp.
aatactagtaagagaaacttctgtgatgtgaggacatttcagccactttatgaactgctggaagcaaaaatgagatctttgcaacatgaagcagttgctcagttcattaaactgtgttcaatatttcagccataacatacattagagaatgatttatattgttcaaacattt

SEQ ID NO: 161 - DNA, 154 bp, Mus sp.
aatactagtaagagaagatttcagccactttatgaactgctggaagcaaaaatgagatctttgcaacatgaagcagttgctcagttcattaaactgtgttcaatatttcagccataacatacattagagaatgatttatattgttcaaacattt

SEQ ID NO: 162 - DNA, 81 bp, Mus sp.
tttaatagcctaagtctggaaattaaatactagtaagagagtgatttcagccactttatgaactgctggaagcaaaaatga

SEQ ID NO: 163 - DNA, 235 bp, Mus sp.
ttagttagaatttaaatattaacaaactataacactatgattaaatgcttgatattgagtagttattttaatagcctaagtctggaaattaaatactagttcagccactttatgaactgctggaagcaaaaatgagatctcattaaactgtgttcaatatttcagccataacatacattagagaatgatttatattgttcaaacatttggtgctctatttttgcatgacgtggga

SEQ ID NO: 164 - DNA, 334 bp, Mus sp.
ttagttagaatttaaatattaacaaactataacactatgattaaatgcttgatattgagtagttattttaatagcctaagtctggaaattaaatactagtaagagaaacttctgtgatgtgaggacatataaagactaatttttttgttgattctaaaaatccacaggtgatttcagccactttatgaactgctggaagcaaaaatgagatctttgcaacatgaagcagttgctcagttcattaaactgtgttcaatatttcagccataacatacattagagaatgatttatattgttcaaacatttggtgctctatttttgcatgacgtggga

SEQ ID NO: 165 - DNA, 203 bp, Mus sp.
ttagttagaatttaaatattaacaaactataacactatgattaaatgcttgatattgagtagttattttaatagcctaagtctggaaattaaatactagtaagagaaacttctgtgatgtgaggacatataaagactaattttttcactccacaggtgatttcagccactttatgaactgctggaagcaaaaatgagatcttt

SEQ ID NO: 166 - DNA, 478 bp, Mus sp.
ttattttaatagcctaagtctggaaattaaatactagtaagagaaacttctgtgatgtgaggacatataaagactaatttttttgttgattctaaaaatcccatgttgtatacttattctttttaaatctgaaaatatattaatcatatattgcctaaatgtcttaataatgtttcactgtaggtaagttaaaatgtatcacatatataataaacatagttattaatgcatagatattcagtaaaattatgacttctaaatttctgtctaaatataatatgccctgtaatataatagaaattattcataagaatacatatatattgctttatcagatattctactttgtttagatctctaaattacataaacttttatttaccttcttcttgatatgaatgaaactcatcaaatatgcgtgttagtgtaaatgaacttctatttaaactccacaggtgatttcagccactttatgaac

SEQ ID NO: 167 - DNA, 703 bp, Mus sp.
ttagttagaatttaaatattaacaaactataacactatgattaaatgcttgatattgagtagttattttaatagcctaagtctggaaattaaatactagtaagagaaacttctgtgatgtgaggacatataaagactaatttttttgttgattctaaaaatcccatgttgtatacttattctttttaaatctgaaaatatattaatcatatattgcctaaatgtcttaataatgtttcactgtaggtaagttaaaatgtatcacatatataataaacatagttattaatgcatagatattcagtaaaattatgacttctaaatttctgtctaaatataatatgccctgtaatataatagaaattattcataagaatacatatatattgctttatcagatattctactttgtttagatctctaaattacataaacttttatttaccttcttcttgatatgaatgaaactcatcaaatatgcgtgttagtgtaaatgaacttctatttaattttgaggctctgcaaagttctccacaggtgatttcagccactttatgaactgctggaagcaaaaatgagatctttgcaacatgaagcagttgctcagttcattaaactgtgttcaatatttcagccataacatacattagagaatgatttatattgttcaaacatttggtgctctatttttgcatgacgtggga

SEQ ID NO: 168 - DNA, 615 bp, Mus sp.
aatactagtaagagaaacttctgtgatgtgaggacatataaagactaatttttttgttgattctaaaaatcccatgttgtatacttattctttttaaatctgaaaatatattaatcatatattgcctaaatgtcttaataatgtttcactgtaggtaagttaaaatgtatcacatatataataaacatagttattaatgcatagatattcagtaaaattatgacttctaaatttctgtctaaatataatatgccctgtaatataatagaaattattcataagaatacatatatattgctttatcagatattctactttgtttagatctctaaattacataaacttttatttaccttcttcttgatatgaatgaaactcatcaaatatgcgtgttagtgtaaatgaacttctatttaattttgaggctctgcaaagttctttgaaagagcaacaaaatggcttcaccactccacaggtgatttcagccactttatgaactgctggaagcaaaaatgagatctttgcaacatgaagcagttgctcagttcattaaactgtgttcaatatttcagccataacatacattagagaatgatttatattgttcaaacattt

SEQ ID NO: 169 - DNA, 776 bp, Mus sp.
ttagttagaatttaaatattaacaaactataacactatgattaaatgcttgatattgagtagttattttaatagcctaagtctggaaattaaatactagtaagagaaacttctgtgatgtgaggacatataaagactaatttttttgttgattctaaaaatcccatgttgtatacttattctttttaaatctgaaaatatattaatcatatattgcctaaatgtcttaataatgtttcactgtaggtaagttaaaatgtatcacatatataataaacatagttattaatgcatagatattcagtaaaattatgacttctaaatttctgtctaaatataatatgccctgtaatataatagaaattattcataagaatacatatatattgctttatcagatattctactttgtttagatctctaaattacataaacttttatttaccttcttcttgatatgaatgaaactcatcaaatatgcgtgttagtgtaaatgaacttctatttaattttgaggctctgcaaagttctttgaaagagcaacaaaatggcttcaactatctgagtgacactgtgaaggagatggccaagaaagcaccttcagaaatatgccatttcagccactttatgaactgctggaagcaaaaatgagatctttgcaacatgaagcagttgctcagttcattaaactgtgttcaatatttcagccataacatacattagagaatgatttatattgttcaaacatttggtgctctatttttgcatgacgtggga

SEQ ID NO: 170 - DNA, 635 bp, Mus sp.
gtctggaaattaaatactagtaagagaaacttctgtgatgtgaggacatataaagactaatttttttgttgattctaaaaatcccatgttgtatacttattctttttaaatctgaaaatatattaatcatatattgcctaaatgtcttaataatgtttcactgtaggtaagttaaaatgtatcacatatataataaacatagttattaatgcatagatattcagtaaaattatgacttctaaatttctgtctaaatataatatgccctgtaatataatagaaattattcataagaatacatatatattgctttatcagatattctactttgtttagatctctaaattacataaacttttatttaccttcttcttgatatgaatgaaactcatcaaatatgcgtgttagtgtaaatgaacttctatttaattttgaggctctgcaaagttctttgaaagagcaacaaaatggcttcaactatctgagtgacactgtgaaggagatggccaagaaagcaccttcagaaatatgccagaaatatctgtcagaatttgaagagattgaggggcactggaagaaactttcctcccagttggtggaaaacaccactccacaggtgatttcagccactttat

SEQ ID NO: 171 - DNA, 714 bp, Mus sp.
tggaaattaaatactagtaagagaaacttctgtgatgtgaggacatataaagactaatttttttgttgattctaaaaatcccatgttgtatacttattctttttaaatctgaaaatatattaatcatatattgcctaaatgtcttaataatgtttcactgtaggtaagttaaaatgtatcacatatataataaacatagttattaatgcatagatattcagtaaaattatgacttctaaatttctgtctaaatataatatgccctgtaatataatagaaattattcataagaatacatatatattgctttatcagatattctactttgtttagatctctaaattacataaacttttatttaccttcttcttgatatgaatgaaactcatcaaatatgcgtgttagtgtaaatgaacttctatttaattttgaggctctgcaaagttctttgaaagagcaacaaaatggcttcaactatctgagtgacactgtgaaggagatggccaagaaagcaccttcagaaatatgccagaaatatctgtcagaatttgaagagattgaggggcactggaagaaactttcctcccagttggtggaaagctgccaaaagctagaagaacatatgaataaacttcgaaaatttcaggtaagccgaggtttggcctttaaactatatttttccactccacaggtgatttcagccactttatgaac

SEQ ID NO: 172 - DNA, 1322 bp, Mus sp.
cctaagtctggaaattaaatactagtaagagaaacttctgtgatgtgaggacatataaagactaatttttttgttgattctaaaaatcccatgttgtatacttattctttttaaatctgaaaatatattaatcatatattgcctaaatgtcttaataatgtttcactgtaggtaagttaaaatgtatcacatatataataaacatagttattaatgcatagatattcagtaaaattatgacttctaaatttctgtctaaatataatatgccctgtaatataatagaaattattcataagaatacatatatattgctttatcagatattctactttgtttagatctctaaattacataaacttttatttaccttcttcttgatatgaatgaaactcatcaaatatgcgtgttagtgtaaatgaacttctatttaattttgaggctctgcaaagttctttgaaagagcaacaaaatggcttcaactatctgagtgacactgtgaaggagatggccaagaaagcaccttcagaaatatgccagaaatatctgtcagaatttgaagagattgaggggcactggaagaaactttcctcccagttggtggaaagctgccaaaagctagaagaacatatgaataaacttcgaaaatttcaggtaagccgaggtttggcctttaaactatattttttcacatagcaattaattggaaaatgtgatgggaaacagatattttacccagagtccttcaaagatattgatgatatcaaaagccaaatctatttcaaaggattgcaacttgcctatttttcctatgaaaacagtaatgtgtcataccttcttggattgtctgtataaatgaattgattttttttcaccaactccaagtatacttaacattttaacataataatttaaaatatccttattccattatgttcattttttaagttgtagatatgatttagctcacagcatacatatatacacatgtattacatatgcatatattatatatatggcagacatatgttttcactaccatatttcacttttgaattatgaatatatgtttaatttctgccatatttccttccctacattgacttctattaatttagtatttcagtagttctaacacattaataataacctagactcaatacagtaatctaacaattatatttgtgcctgtaattctaagttagttaaattcataggttgtgtttctcatagttggccatttgtgaaatataataatatccgaaaagaaagttcaaaaatgtcatgacttcatatagacaggtgatttcagccactttatg

SEQ ID NO: 173 - DNA, 6948 bp, Artificial Sequence
  Description of Artificial Sequence Synthetic polynucleotide
gggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatcttcaatattgggtattagtcatcgctattaccatgatgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctagcgcagtcagtgcttctgacacaacagtctcgaacttaagctgcagaagttggtcgtgaggcactgggcaggtaagtatcaaggttacaagacaggtttaaggacaccaatagaaactgggcttgtcgagacagagaagactcttgcgtttctgataggcacctattggtcttactgacatccactttgcctttctctccacaggtaattgtgagcggataacaattgatgtcgcacaggccacggattaggcaccccaggcttgacactttatgcttccggctcgtatattgtgtggaattgtgagcggataacaatttcacacaggagatatatatatgggctaggccaccatggcaccgaagaagaagcgcaaggtgcatatgaatacaaaatataataaagagttcttactctacttagcagggtttgtagacggtgacggttccatctttgcctgtatccatcctagtcaagcgtataagttcaagcaccggctgactctccatttcacggtcactcagaagacacagcgccgttggttcctcgacaagctggtggacgagatcggtgtgggttacgtgcaggacgtgggcagcgtctcccagtaccggctgtcccagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacgcgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctctgccactatcgctccggctcagtatggtaagttcaagcactatctggggctccggttctatgtcagtcagaagacacagcgccgttggttcctcgacaagctggtggacgagatcggtgtgggttacgtgagtgaccagggcagcgtctccaggtactgtctgtcccagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccctaaggtaccagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttgtattagtcatcgctattaccatgatgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctagcgcagtcagtgcttctgacacaacagtctcgaacttaagctgcagaagttggtcgtgaggcactgggcaggtaagtatcaaggttacaagacaggtttaaggacaccaatagaaactgggcttgtcgagacagagaagactcttgcgtttctgataggcacctattggtcttactgacatccactttgcctttctctccacaggtaattgtgagcggataacaattgatgtcgcacaggccacggattaggcaccccaggcttgacactttatgcttccggctcgtatattgtgtggaattgtgagcggataacaatttcacacaggagatatatatatgggctaggccaccatggcaccgaagaagaagcgcaaggtgcatatgaatacaaaatataataaagagttcttactctacttagcagggtttgtagacggtgacggttccatctatgcctgtatcaggccgacgcagagtgtgaagttcaagcacgatctgctgctctgtttcgatgtctctcagaagacacagcgccgttggttcctcgacaagctggtggacgagatcggtgtgggttacgtgtatgaccgtggcagcgtctcctcgtacaggctgtccgagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacgcgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctgggcctcgatcgagcctaggcaacagtctaagttcaagcaccagctgcggctcgggttctcggtctatcagaagacacagcgccgttggttcctcgacaagctggtggacgagatcggtgtgggttacgtgcgtgacactggcagcgtctcctgttactgtctgtcccagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccctaaggtaccagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagcctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcgctattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatactttcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgcggataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgata

SEQ ID NO: 174 - DNA, 35 bp, Mus sp.
catttcatatttagtgacataagatatgaagtatg

SEQ ID NO: 175 - DNA, 27 bp, Mus sp.
gtgtcagtaatctctatccctttcatg

SEQ ID NO: 176 - DNA, 330 bp, Mus sp.
catttcatatttagtgacataagatatgaagtatgattattcagccactttatgaactgctggaagcaaaaatgagatctttgcaacatgaagcagttgctcagttcattaaactgtgttcaatatttcagccataacatacattagagaatgatttatattgttcaaacatttggtgctctatttttgcatgacgtgggattaaacacagcaccaacaatcaaacaattgcaaagatgtattacaagtattttttctttttaaaacaggaaagtatacttatatttccattgtccaaaccatcatgaaagggatagagattactgacac

SEQ ID NO: 177 - DNA, 381 bp, Mus sp.
catttcatatttagtgacataagatatgaagtatgattattaaaattaaatcacattattttattataattactttactccacaggtgatttcagccactttatgaactgctggaagcaaaaatgagatctttgcaacatgaagcagttgctcagttcattaaactgtgttcaatatttcagccataacatacattagagaatgatttatattgttcaaacatttggtgctctatttttgcatgacgtgggattaaacacagcaccaacaatcaaacaattgcaaagatgtattacaagtattttttctttttaaaacaggaaagtatacttatatttccattgtccaaaccatcatgaaagggatagagattactgacac

SEQ ID NO: 178 - DNA, 378 bp, Mus sp.
catttcatatttagtgacataagatatgaagtatgattattaaaattaaatcacattattttattataattactttacacaggtgatttcagccactttatgaactgctggaagcaaaaatgagatctttgcaacatgaagcagttgctcagttcattaaactgtgttcaatatttcagccataacatacattagagaatgatttatattgttcaaacatttggtgctctatttttgcatgacgtgggattaaacacagcaccaacaatcaaacaattgcaaagatgtattacaagtattttttctttttaaaacaggaaagtatacttatatttccattgtccaaaccatcatgaaagggatagagattactgacac

SEQ ID NO: 179 - DNA, 1083 bp, Artificial Sequence
  Description of Artificial Sequence Synthetic polynucleotide
gaaagaaaatgccaatagtccaaaatagttgctttatctttcttttaatcaataaatatattcattttaaagggaaaaattgcaaccttccatttaaaatcagcttttatattgagtatttttttaaaatgttgtgtgtacatgctaggtgtgtatattaatttttatttgttacttgaaactaaactctgcaaatgcaggaaactatcagagtgatatctttgtcagtataaccaaaaaatatacgctatatctctataatctgttttacataatccatctatttttcttgatccatatgcttttacctgcaggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggtaagtctttgatttgttttttcgaaattgtatttatcttcagcacatctggactctttaacttcttaaagatcaggttctgaagggtgatggaaattacttttgactgttgttgtcatcattatattactagaaagaaaattatcataatgataatattagagcacggtgctatggactttttgtgtcaggatgagagagtttgcctggacggagctggtttatctgataaactgcaaaatataattgaatctgtgacagagggaagcatcgtaacagcaaggtgttttgtggctttggggcagtgtgtatttcggctttatgttggaacctttccagaaggagaacttgtggcatacttagctaaaatgaagttgctagaaatatccatcatgataaaattacagttctgttttcctaaagacaattttgtagtgctgtagcaatatttctatatattctattgacaaaatgccttctgaaatagtccagaggccaaaacaatgcagagttaattgttggtacttattgacattttatggtttatgttaatagggaaacagcatatggatgataaccagtgtgtagtttaatttcaacttgtggtgtcctttgaatatg

SEQ ID NO: 180 - DNA, 875 bp, Homo sapiens
  modified_base (29)..(846): a, c, t, g, unknown or other
aaaatgttgtgtgtacatgctaggtgtgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagttaattgttggtacttattgacatttt

SEQ ID NO: 181 - DNA, 29 bp, Artificial Sequence
  Description of Artificial Sequence Synthetic oligonucleotide
aaaatgttgtgtgtacttattgacatttt

SEQ ID NO: 182 - DNA, 29 bp, Artificial Sequence
  Description of Artificial Sequence Synthetic oligonucleotide
aaaatgttgtgtgtacttattgacatttt

SEQ ID NO: 183 - DNA, 19 bp, Artificial Sequence
  Description of Artificial Sequence Synthetic oligonucleotide
aaaatgttattgacatttt

SEQ ID NO: 184 - DNA, 1384 bp, Artificial Sequence
  Description of Artificial Sequence Synthetic polynucleotide
ctaaccgagagggtgcttttttccctgacacataaaaggtgtctttctgtcttgtatcctttggatatgggcatgtcagtttcatagggaaattttcacatggagcttttgtatttctttctttgccagtacaactgcatgtggtagcacactgtttaatcttttctcaaataaaaagacatggggcttcatttttgttttgcctttttggtatcttacaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggtagggcgacagatctaataggaatgaaaacattttagcagactttttaagctttctttagaagaatatttcatgagagattataagcagggtgaaaggcactaacattaaagaacctatcaaccattaatcaacagcagtaaagaaattttttatttctttttttcatatactaaaatatatacttgtggctagttagtggttttctgctattttaaacttgaagtttgctttaaaaatcacccatgattgcttaaaggtgaatatcttcaatatattttaacttcaacaagctgaatctcagttgtttttcaagaagattttagaaagcaattataaatgattgttttgtaggaaagacagatctttgcttagttttaaaaatagctatgaatatgactatgaagctaaaaaaagtgatagtgtcacttacctctagtttcaccacatttgtgaatacattcttgaaggggaacttgagccaaagaggtacaagtttaatggggaaaacaaaacctcaaaaaggttactgtcaaattcaatcatcatttaaatttcccttggaatgtattgaaggcacagaaagccaaatgcgtgctgctgcagttggaaagcctagagagtttataaatgggattttgtattatgcttccagttgttgatgttaatgtgtcttgtttcgtaaaggaagacttggcctttatttaccaaatgagactattgttatgaacaatgaaaacttcgttcttttgccaagctcttgcatcccacccatcatccacataataggtggattttaatattcaggaagctagaacaactcattgatgaatatctttcgttaagatgtattaaaaagaagattttggaattatgtcagttgtctttgcccacctcctctttccctctttattcatgttacattattcagaaagtagatacaattcatattttgtacaaaataaacacattagttgacctaaacac

SEQ ID NO: 185 - DNA, 1253 bp, Homo sapiens
  modified_base (29)..(1223): a, c, t, g, unknown or other
ctttctttgccagtacaactgcatgtggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaattcatattttgtacaaaataaacacatt

SEQ ID NO: 186 - DNA, 30 bp, Artificial Sequence
  Description of Artificial Sequence Synthetic oligonucleotide
ctttctttgccagtacaaaataaacacatt

SEQ ID NO: 187 - DNA, 15 bp, Artificial Sequence
  Description of Artificial Sequence Synthetic oligonucleotide
caaaataaacacatt

SEQ ID NO: 188 - protein, 9 aa, Homo sapiens
LAGLIDADG

(19) United States
(12) Patent Application Publication
(10) Pub. No.: US 2017/0106055 A1
(43) Pub. Date: Apr. 20, 2017

(54) Dystrophin Gene Exon Deletion Using Engineered Nucleases

(71) Applicant: Precision Biosciences, Inc., Durham, NC (US)

(72) Inventors: Derek JANTZ, Durham, NC (US); James Jefferson SMITH, Durham, NC (US); Michael G. NICHOLSON, Chapel Hill, NC (US)

(21) Appl. No.: 15/124,984

(22) PCT Filed: Mar. 12, 2015
(86) PCT No.: PCT/US15/20205

§ 371 Date: Sep. 9, 2016

Related U.S. Application Data
(60) Provisional application No. 61/951,648, filed on Mar. 12, 2014.

(51) Int. Cl. A61K 38/46; C12N 9/22; C12N 7/00

(52) U.S. Cl. CPC A61K 38/465; C12N 7/00; C12N 9/22; C12N 2750/14143

(57) ABSTRACT

The invention relates to the field of molecular biology and recombinant nucleic acid technology. In particular, the invention relates to methods of treating patients with Duchenne Muscular Dystrophy comprising the removal of at least one exon from the dystrophin gene using engineered nucleases to restore the normal reading frame. Further disclosed are engineered nucleases suitable for using the methods.

### RELATED APPLICATIONS

This application claims benefit of priority to U.S. Provisional Patent Application No. 61/951,648, filed Mar. 12, 2014, the entire disclosure of which is hereby incorporated by reference.

### FIELD OF THE INVENTION

The invention relates to the field of molecular biology and recombinant nucleic acid technology. In particular, the invention relates to a method of treating a patient with Duchenne Muscular Dystrophy comprising the removal of at least one exon from the dystrophin gene using engineered nucleases.

### BACKGROUND OF THE INVENTION

Duchenne Muscular Dystrophy is a rare, X-linked muscle degenerative disorder that affects about 1 in every 3500 boys worldwide. The disease is caused by mutations in the dystrophin (DMD) gene, which is the largest known gene. DMD spans 2.2 Mb of the X chromosome and encodes predominantly a 14-kb transcript derived from 79 exons. The full-length dystrophin protein, as expressed in skeletal muscle, smooth muscle, and cardiomyocytes, is 3685 amino acids and has a molecular weight of 427 kD. The severe Duchenne phenotype is generally associated with the loss of full length dystrophin protein from skeletal and cardiac muscle, which leads to debilitating muscle degeneration and, ultimately, heart failure. A large number of different DMD mutations have been described, many of them resulting in either the severe Duchenne Muscular Dystrophy or the milder Becker Muscular Dystrophy. The Leiden University Medical Center maintains a database of mutations in the DMD gene (http://www.dmd.nl).

There are several therapeutic strategies being pursued for the treatment of Duchenne Muscular Dystrophy. First, “gene replacement” strategies are an active area of research (Oshima, et al. (2009) J of the Am. Soc. of Gene Ther. 17:73-80; Liu, et al. (2005) Mol. Ther. 11:245-256; Lai, et al. (2006) Hum Gene Ther. 17:1036-1042; Odom et al. (2008) Mol. Ther. 16:1539-1545). This approach involves delivering a functional copy of the DMD gene to patients using a viral delivery vector, typically adeno-associated virus (AAV). The large size of the DMD gene makes it incompatible with the limited carrying capacity of common viral vectors, however. This necessitates the use of a “micro-dystrophin” gene in which most of the repetitive central portion of the gene is removed to leave only the minimal functional protein. It is not clear, however, that expression of “micro-dystrophin” is sufficient for clinical benefit. In addition, this approach suffers from the possibility of random gene integration into the patient genome, which could lead to insertional mutagenesis, and the potential for immune reactions against the delivery vector.

A second approach to treating Duchenne Muscular Dystrophy involves the transplantation of healthy muscle precursor cells into patient muscle fibres (Peault et al. (2007) Mol. Ther. 15:867-877; Skuk, et al. (2007) Neuromuscul. Disord. 17:38-46). This approach suffers from inefficient migration of the transplanted myoblasts and the potential for immune rejection by the patient.

A third approach involves suppression of nonsense mutations using PTC 124 (Welch, et al. (2007) Nature 447:87-91). This would require lifelong dosing of the drug, however, and the approach is yet to show any significant clinical benefit.

A fourth, and more promising, potential treatment for Duchenne Muscular Dystrophy is called “Exon Skipping” (Williams, et al. (2008) BMC Biotechnol. 8:35; Jearawiriyapaisarn et al. (2008) Mol Ther. 16:1624-1629; Yokota, et al. (2007) Acta Myol. 26:179-184; van Deutekom et al. (2001) Hum. Mol. Gen. 10:1547-1554; Benedetti et al. (2013) FEBS J. 280:4263-80; Rodino-Klapac (2013) Curr Neurol Neurosci Rep. 13:332; Verhaart and Aartsma-Rus (2012) Curr Opin Neurol. 25:588-96). In general, the N- and C-terminal portions of the dystrophin gene are essential for its role as a “scaffold” protein that maintains membrane integrity in muscle fibres whereas the central “rod domain”, which comprises 24 spectrin-like repeats, is at least partially dispensible. Indeed, the severe Duchenne phenotype is typically associated with mutations in the dystrophin gene that introduce frameshifts and/or premature termination codons, resulting in a truncated form of the dystrophin protein lacking the essential C-terminal domain. Mutations in the central rod domain, including large deletions of whole exons, typically result in the much milder Becker phenotype if they maintain the reading frame such that the C-terminal domain of the protein is intact.

Duchenne Muscular Dystrophy is most frequently caused by the deletion of one or more whole exon(s), resulting in reading frame shift. For example, Exon 45 is frequently deleted in Duchenne patients. Because Exon 45 is 176 bp long, which is not divisible by three, deleting the exon shifts Exons 46-79 into the wrong reading frame. The same can be said of Exon 44, which is 148 bp in length. However, if Exons 44 and 45 are deleted, the total size of the deletion is 324 bp, which is divisible by three. Thus, the deletion of both exons does not result in a reading frame shift. Because these exons encode a portion of the non-essential rod domain of the dystrophin protein, deleting them from the protein is expected to result in a mild Becker-like phenotype. Thus, a patient with the Duchenne phenotype due to the deletion of one or more exon(s) can, potentially, be treated by eliminating one or more adjacent exons to restore the reading frame. This is the principle behind “Exon Skipping,” in which modified oligonucleotides are used to block splice acceptor sites in dystrophin pre-mRNA so that one or more specific exons are absent from the processed transcript. The approach has been used to restore dystrophin gene expression in the mdx mouse model by skipping Exon 23, which harbored a disease-inducing nonsense mutation (Mann, et al. (2001) Proc. Nat. Acad. Sci. USA 98:42-47). Oligonucleotide analogs which induce skipping of Exon 51 have also shown promise in early human clinical trials (Benedetti et al. (2013) FEBS J. 280:4263-80). The major limitations with this approach are: (1) the exon-skipping process is inefficient, resulting in relatively low levels of functional dystrophin expression; and (2) the exon-skipping oligonucleotide has a relatively short half-life so the affect is transient, necessitating repeated and life-long dosing. Thus, while Exon-Skipping approaches have shown some promise in clinical trials, the improvements in disease progression have been minimal and variable.

The present invention improves upon current Exon-Skipping approaches by correcting gene expression at the level of the genomic DNA rather than pre-mRNA. The invention is a permanent treatment for Duchenne Muscular Dystrophy that involves the excision of specific exons from the DMD coding sequence using a pair of engineered, site-specific endonucleases. By targeting a pair of such endonucleases to sites in the intronic regions flanking an exon, it is possible to permanently remove the intervening fragment containing the exon from the genome. The resulting cell, and its progeny, will express mutant dystrophin in which a portion of the non-essential spectrin repeat domain is removed but the essential N- and C-terminal domains are intact.

Methods for producing engineered, site-specific endonucleases are known in the art. For example, zinc-finger nucleases (ZFNs) can be engineered to recognize and cut pre-determined sites in a genome. ZFNs are chimeric proteins comprising a zinc finger DNA-binding domain fused to the nuclease domain of the FokI restriction enzyme. The zinc finger domain can be redesigned through rational or experimental means to produce a protein which binds to a pre-determined DNA sequence ˜18 basepairs in length. By fusing this engineered protein domain to the FokI nuclease, it is possible to target DNA breaks with genome-level specificity. ZFNs have been used extensively to target gene addition, removal, and substitution in a wide range of eukaryotic organisms (reviewed in S. Durai et al., Nucleic Acids Res 33, 5978 (2005)). Likewise, TAL-effector nucleases (TALENs) can be generated to cleave specific sites in genomic DNA. Like a ZFN, a TALEN comprises an engineered, site-specific DNA-binding domain fused to the FokI nuclease domain (reviewed in Mak, et al. (2013) Curr Opin Struct Biol. 23:93-9). In this case, however, the DNA binding domain comprises a tandem array of TAL-effector domains, each of which specifically recognizes a single DNA basepair. A limitation that ZFNs and TALENs have for the practice of the current invention is that they are heterodimeric, so that the production of a single functional nuclease in a cell requires co-expression of two protein monomers. Because the current invention requires two nucleases, one to cut on either side of the exon of interest, this would necessitate co-expressing four ZFN or TALEN monomers in the same cell. This presents significant challenges in gene delivery because traditional gene delivery vectors have limited carrying capacity. It also introduces the possibility of “mis-dimerization” in which the monomers associate inappropriately to make unintended dimeric endonuclease species that might recognize and cut off-target locations in the genome. This can, potentially, be minimized by generating orthogonal obligate heterodimers in which the FokI nuclease domains of the four monomers are differentially engineered to dimerize preferentially with the intended partner monomer.

Compact TALENs are an alternative endonuclease architecture that avoids the need for dimerization (Beurdeley, et al. (2013) Nat Commun. 4:1762). A Compact TALEN comprises an engineered, site-specific TAL-effector DNA-binding domain fused to the nuclease domain from the I-TevI homing endonuclease. Unlike FokI, I-TevI does not need to dimerize to produce a double-strand DNA break so a Compact TALEN is functional as a monomer. Thus, it is possible to co-express two Compact TALENs in the same cell to practice the present invention.

Engineered endonucleases based on the CRISPR/Cas9 system are also know in the art (Ran, et al. (2013) Nat Protoc. 8:2281-2308; Mali et al. (2013) Nat Methods. 10:957-63). A CRISPR endonuclease comprises two components: (1) a caspase effector nuclease, typically microbial Cas9; and (2) a short “guide RNA” comprising a ˜20 nucleotide targeting sequence that directs the nuclease to a location of interest in the genome. By expressing multiple guide RNAs in the same cell, each having a different targeting sequence, it is possible to target DNA breaks simultaneously to multiple sites in in the genome. Thus, CRISPR/Cas9 nucleases are suitable for the present invention. The primary drawback of the CRISPR/Cas9 system is its reported high frequency of off-target DNA breaks, which could limit the utility of the system for treating human patients (Fu, et al. (2013) Nat Biotechnol. 31:822-6).

In the preferred embodiment of the invention, the DNA break-inducing agent is an engineered homing endonuclease (also called a “meganuclease”). Homing endonucleases are a group of naturally-occurring nucleases which recognize 15-40 base-pair cleavage sites commonly found in the genomes of plants and fungi. They are frequently associated with parasitic DNA elements, such as group 1 self-splicing introns and inteins. They naturally promote homologous recombination or gene insertion at specific locations in the host genome by producing a double-stranded break in the chromosome, which recruits the cellular DNA-repair machinery (Stoddard (2006), Q. Rev. Biophys. 38: 49-95). Homing endonucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif (see Chevalier et al. (2001), Nucleic Acids Res. 29(18): 3757-3774). The LAGLIDADG homing endonucleases with a single copy of the LAGLIDADG motif form homodimers, whereas members with two copies of the LAGLIDADG motif are found as monomers.

I-CreI (SEQ ID NO: 1) is a member of the LAGLIDADG family of homing endonucleases which recognizes and cuts a 22 basepair recognition sequence in the chloroplast chromosome of the algae Chlamydomonas reinhardtii. Genetic selection techniques have been used to modify the wild-type I-CreI cleavage site preference (Sussman et al. (2004), J. Mol. Biol. 342: 31-41; Chames et al. (2005), Nucleic Acids Res. 33: e178; Seligman et al. (2002), Nucleic Acids Res. 30: 3870-9, Arnould et al. (2006), J. Mol. Biol. 355: 443-58). More recently, a method of rationally-designing mono-LAGLIDADG homing endonucleases was described which is capable of comprehensively redesigning I-CreI and other homing endonucleases to target widely-divergent DNA sites, including sites in mammalian, yeast, plant, bacterial, and viral genomes (WO 2007/047859).

As first described in WO 2009/059195, I-CreI and its engineered derivatives are normally dimeric but can be fused into a single polypeptide using a short peptide linker that joins the C-terminus of a first subunit to the N-terminus of a second subunit (Li, et al. (2009) Nucleic Acids Res. 37:1650-62; Grizot, et al. (2009) Nucleic Acids Res. 37:5405-19.) Thus, a functional “single-chain” meganuclease can be expressed from a single transcript. By delivering genes encoding two different single-chain meganucleases to the same cell, it is possible to simultaneously cut two different sites. This, coupled with the extremely low frequency of off-target cutting observed with engineered meganucleases makes them the preferred endonuclease for the present invention.

The use of engineered meganucleases for treatment of Duchenne Muscular Dystrophy was previously disclosed in WO 2011/141820 (the '820 application). In this patent application, the authors discuss the possibility of using engineered meganucleases to correct defects in the DMD gene via three different mechanisms (see WO 2011/141820 FIG. 1). First, the authors contemplate the use of an engineered meganuclease to insert a transgenic copy of DMD or micro-DMD into a “safe harbor” locus, such as AAVS1, where it will be expressed constitutively without affecting endogenous gene expression. Second, the authors propose that a meganuclease might be made to cleave the genome at a site near a deleterious mutation in DMD and that this DNA break would stimulate homologous recombination between the mutant DMD gene in the genome and a healthy copy of the gene provided in trans such that the mutation in the genome would be corrected. Third, the authors of the '820 application propose that an engineered meganuclease can be made to insert foreign DNA into an intron in the DMD gene and that such a meganuclease could be used insert the essential C-terminal domain of dystrophin into an early intron upstream of a mutation causing disease. Significantly, in contemplating the myriad uses of meganucleases for manipulating the DMD gene, the authors of the '820 application do not contemplate the use of two meganucleases simultaneously in the same cells, nor do they propose the removal of any DNA sequence as in the present invention.

Finally, Ousterout et al. demonstrated that a DNA break can be targeted to the DMD coding sequence using a TALEN and that the break is frequently repaired via the mutagenic non-homologous end joining pathway, resulting in the introduction of small insertions and/or deletions (“indels”) that can change the reading frame of the gene (Ousterout et al. (2013) Mol Ther. 21:1718-26). They demonstrated the possibility of restoring DMD gene expression in a portion of mutant cells by delivering a DNA break to the exon immediately following the mutation and relying on mutagenic DNA repair to restore the reading frame in some percentage of cells. Unlike the present invention, this approach involved a single nuclease and was targeted to the coding sequence of the gene.

### SUMMARY OF THE INVENTION

The present invention is a method of treating Duchenne Muscular Dystrophy comprising delivering a pair of engineered nucleases, or genes encoding engineered nucleases, to the muscle cells of a patient such that the two nucleases excise one or more exons of the DMD gene to restore the normal reading frame. Cells so treated will express a shortened form of the dystrophin protein in which a portion of the central spectrin repeat domain is absent but the N- and C-terminal domains are intact. This will, in many cases, reduce the severity of the disease to the mild Becker phenotype.

Thus, in one embodiment, the invention provides a general method for treating Duchenne Muscular Dystrophy using a pair of nucleases. In another embodiment, the invention provides engineered meganucleases suitable for practicing the method. In a third embodiment, the invention provides engineered Compact TALENs suitable for practicing the method. In a fourth embodiment, the invention provides CRISPRs for practicing the method. In a fifth embodiment, the invention provides vectors and techniques for delivering engineered nucleases to patient cells.

### BRIEF DESCRIPTION OF THE FIGURES

FIG. 1. Structure of the DMD gene. 79 exons are drawn to indicate reading frame. The essential Actin-binding and Dystroglycan-binding domains, which span approximately Exons 2-8 and 62-70, respectively, are indicated.

FIG. 2. Strategies for deleting exons from the DMD gene using different types of nucleases. 2A) Strategy for deleting an exon using a pair of CRISPRs. A pair of “guide RNAs” (“gRNAs”) are used which are complementary to a pair of recognition sites flanking the exon of interest. As drawn in this figure, the gRNAs can be complementary to recognition sequences that are distal to the conserved “GG” motif and the site of Cas9 DNA cleavage. In this orientation, the CRISPR recognition sequences are largely conserved following DNA cleavage, excision of the intervening fragment of genomic DNA, and re-joining of the chromosome ends. 2B) An alternative scheme for deleting an exon using a pair of CRISPRs in which the gRNAs are complementary to recognition sequences that are proximal to the exon. In this orientation, the CRISPR recognition sequences are largely deleted following DNA cleavage, excision of the intervening fragment of genomic DNA, and re-joining of the chromosome ends. It is contemplated in the invention could also comprise a hybrid of the schemes shown in 2A and 2B. 2C) Strategy for deleting an exon using a pair of compact TALENs (cTALENs). A pair of TAL effector DNA-binding domains (“TALEs”) are used which bind to a pair of recognition sites flanking the exon of interest. As drawn in this figure, the TALEs can bind to recognition sequences that are distal to the conserved “CNNNG” motif that is recognized and cut by the I-TevI cleavage domain (“TevI-CD”). In this orientation, the cTALEN recognition sequences are largely conserved following DNA cleavage, excision of the intervening fragment of genomic DNA, and re-joining of the chromosome ends. Also, the cTALENs in this figure are shown with the TALE and TevI-CD domains in an N- to C-orientation. It is also possible to generate cTALENs with these two domains in a C- to N-orientation. 2D) An alternative scheme for deleting an exon using a pair of cTALENS in which the TALE domains bind to recognition sequences that are proximal to the exon. In this orientation, the cTALEN recognition sequences are largely deleted following DNA cleavage, excision of the intervening fragment of genomic DNA, and re-joining of the chromosome ends. Also, the cTALENs in this figure are drawn with the TALE and TevI-CD domains in a C- to N-orientation. It is contemplated in the invention could also comprise a hybrid of the schemes shown in 2C and 2D. 2E) Strategy for deleting an exon from the DMD gene using a pair of single-chain meganucleases. The meganucleases are drawn as two-domain proteins (MGN-N: the N-terminal domain; and MGN-C: the C-terminal domain) joined by a linker. In the figure, the C-terminal domain is drawn as binding to the half of the recognition sequence that is closest to the exon. In some embodiments, however, the N-terminal domain can bind to this half of the recognition sequence. The central four basepairs of the recognition sequence are shown as “NNNN”. These four basepairs become single-strand 3′ “overhangs” following cleavage by the meganuclease. The subset of preferred four basepair sequences that comprise this region of the sequence are identified in WO/2010/009147. DNA cleavage by the pair of meganucleases generates a pair of four basepair 3′ overhangs at the chromosome ends. If these overhangs are complementary, they can anneal to one another and be directly re-ligated, resulting in the four basepair sequence being retained in the chromosome following exon excision. Because meganucleases cleave near the middle of the recognition sequence, half of each recognition sequence will frequently be retained in the chromosome following excision of the exon. The other half of each recognition sequence will removed from the genome with the exon.

FIG. 3. Excision of DMD Exon 44 using the DYS-1/2 and DYS-3/4 meganucleases. 3A) Sequence of DMD Exon 44 and flanking regions. The Exon sequence is underlined. Recognition sites for the DYS-1/2 and DYS-3/4 meganucleases are shaded in gray with the central four basepairs (which become the 3′ overhang following cleavage by the meganuclease) in bold. Annealing sites for a pair of PCR primers used for analysis are italicized. 3B) Agarose gel electrophoresis analysis of HEK-293 cells co-expressing DYS-1/2 and DYS-3/4. Genomic DNA was isolated from the cells and evaluated by PCR using the primers indicated in (3A). PCR products were resolved on an agarose gel and it was found that HEK-293 cells co-expressing the two meganucleases yielded a pair of PCR bands whereas wild-type HEK-293 cells yielded only the larger band. 3C) sequences from three plasmids harboring the smaller PCR product from (3B). The three sequences are shown aligned to the wild-type human sequence. The locations of the DYS-1/2 and DYS-3/4 recognition sequences are shaded in gray with the central four basepairs in bold.

FIG. 4. Excision of DMD Exon 45 using the DYS-5/6 and DYS-7/8 meganucleases. 4A) Sequence of DMD Exon 45 and flanking regions. The Exon sequence is underlined. Recognition sites for the DYS-5/6 and DYS-7/8 meganucleases are shaded in gray with the central four basepairs (which become the 3′ overhang following cleavage by the meganuclease) in bold. Annealing sites for a pair of PCR primers used for analysis are italicized. 4B) Agarose gel electrophoresis analysis of HEK-293 cells co-expressing DYS-5/6 and DYS-7/8. Genomic DNA was isolated from the cells and evaluated by PCR using the primers indicated in (4A). PCR products were resolved on an agarose gel and it was found that HEK-293 cells co-expressing the two meganucleases yielded a pair of PCR bands whereas wild-type HEK-293 cells yielded only the larger band. 4C) sequences from 16 plasmids harboring the smaller PCR product from (4B). The sequences are shown aligned to the wild-type human sequence. The locations of the DYS-5/6 and DYS-7/8 recognition sequences are shaded in gray with the central four basepairs in bold.

FIG. 5. Evaluation of the MDX-1/2 and MDX-13/14 meganucleases in a reporter assay in CHO cells. A) Schematic of the assay. For each of the two meganucleases, we produced a CHO cell line in which a reporter cassette was integrated stably into the genome of the cell. The reporter cassette comprised, in 5′ to 3′ order: an SV40 Early Promoter; the 5′ 2/3 of the GFP gene; the recognition site for either MDX-1/2 (SEQ ID NO: 149) or the recognition site for MDX-13/14 (SEQ ID NO: 150); the recognition site for the CHO-23/24 meganuclease (WO/2012/167192); and the 3′ 2/3 of the GFP gene. Cells stably transfected with this cassette did not express GFP in the absence of a DNA break-inducing agent. When a DNA break was induced at either of the meganuclease recognition sites, however, the duplicated regions of the GFP gene recombined with one another to produce a functional GFP gene. The percentage of GFP-expressing cells could then be determined by flow cytometry as an indirect measure of the frequency of genome cleavage by the meganucleases. B,C) The two CHO reporter lines were transfected with mRNA encoding the MDX-1/2 (A), MDX-13/14 (B), or CHO-23/34 (A and B) meganucleases. 1.5e6 CHO cells were transfected with 1e6 copies of mRNA per cell using a Lonza Nucleofector 2 and program U-024 according to the manufacturer's instructions. 48 hours post-transfection, the cells were evaluated by flow cytometry to determine the percentage of GFP-positive cells compared to an untransfected (Empty) negative control. The assay was performed in triplicate and standard deviations are shown. The MDX-1/2 and MDX-13/14 meganucleases were found to produce GFP+ cells in their respective cell lines at frequencies significantly exceeding both the negative (Empty) control and the CHO-23/24 postive control, indicating that the nucleases are able to efficiently recognize and cut their intended target sequences in a cell.

FIG. 6. Sequence alignments from 20 C2C12 mouse myoblast clones in which a portion of the DMD gene was deleted by co-transfection with the MDX-1/2 and MDX-13/14 meganucleases. The location of DMD Exon 23 is shown as are the locations and sequences of the MDX-1/2 and MDX-13/14 target sites. Each of the 20 sequences (SEQ ID NO: 153-172) was aligned to a reference wild-type DMD sequence and deletions relative to the reference are shown as hollow bars.

FIG. 7. Vector map of the pAAV-MDX plasmid. This “packaging” plasmid was used in conjunction with an Ad helper plasmid to produce AAV virus capable of simultaneously delivering the genes encoding the MDX-1/2 and MDX-13/14 meganucleases.

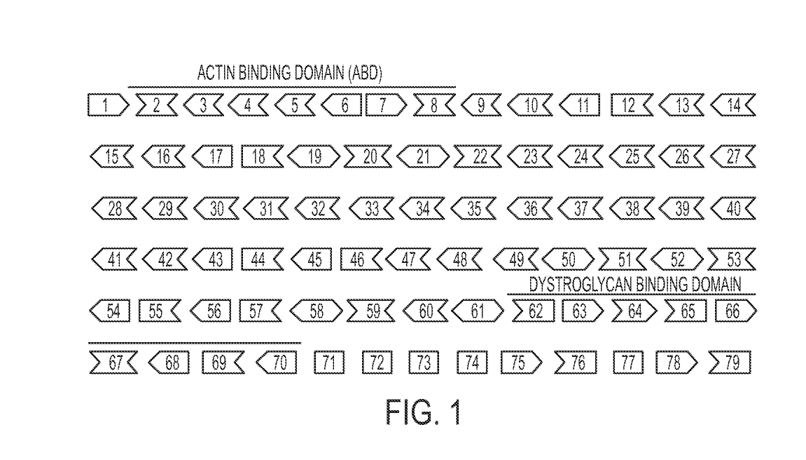

### DETAILED DESCRIPTION OF THE INVENTION

### 1.1 References and Definitions

The patent and scientific literature referred to herein establishes knowledge that is available to those of skill in the art. The issued U.S. patents, allowed applications, published foreign applications, and references, including GenBank database sequences, that are cited herein are hereby incorporated by reference to the same extent as if each was specifically and individually indicated to be incorporated by reference.

As used herein, the term “meganuclease” refers to an endonuclease that is derived from I-CreI. The term meganuclease, as used herein, refers to an engineered variant of I-CreI that has been modified relative to natural I-CreI with respect to, for example, DNA-binding specificity, DNA cleavage activity, DNA-binding affinity, or dimerization properties. Methods for producing such modified variants of I-CreI are known in the art (e.g. WO 2007/047859). A meganuclease may bind to double-stranded DNA as a homodimer, as is the case for wild-type I-CreI, or it may bind to DNA as a heterodimer. A meganuclease may also be a “single-chain meganuclease” in which a pair of DNA-binding domains derived from I-CreI are joined into a single polypeptide using a peptide linker.

As used herein, the term “single-chain meganuclease” refers to a polypeptide comprising a pair of meganuclease subunits joined by a linker. A single-chain meganuclease has the organization: N-terminal subunit—Linker—C-terminal subunit. The two meganuclease subunits, each of which is derived from I-CreI, will generally be non-identical in amino acid sequence and will recognize non-identical DNA sequences. Thus, single-chain meganucleases typically cleave pseudo-palindromic or non-palindromic recognition sequences. A single chain meganuclease may be referred to as a “single-chain heterodimer” or “single-chain heterodimeric meganuclease” although it is not, in fact, dimeric. For clarity, unless otherwise specified, the term “meganuclease” can refer to a dimeric or single-chain meganuclease.

As used herein, the term “Compact TALEN” refers to an endonuclease comprising a DNA-binding domain with 16-22 TAL domain repeats fused in any orientation to any portion of the I-TevI homing endonuclease.

As used herein, the term “CRISPR” refers to a caspase-based endonuclease comprising a caspase, such as Cas9, and a guide RNA that directs DNA cleavage of the caspase by hybridizing to a recognition site in the genomic DNA.

As used herein, with respect to a protein, the term “recombinant” means having an altered amino acid sequence as a result of the application of genetic engineering techniques to nucleic acids which encode the protein, and cells or organisms which express the protein. With respect to a nucleic acid, the term “recombinant” means having an altered nucleic acid sequence as a result of the application of genetic engineering techniques. Genetic engineering techniques include, but are not limited to, PCR and DNA cloning technologies; transfection, transformation and other gene transfer technologies; homologous recombination; site-directed mutagenesis; and gene fusion. In accordance with this definition, a protein having an amino acid sequence identical to a naturally-occurring protein, but produced by cloning and expression in a heterologous host, is not considered recombinant.

As used herein, the term “wild-type” refers to any naturally-occurring form of a meganuclease. The term “wild-type” is not intended to mean the most common allelic variant of the enzyme in nature but, rather, any allelic variant found in nature. Wild-type homing endonucleases are distinguished from recombinant or non-naturally-occurring meganucleases.

As used herein, the term “recognition sequence” refers to a DNA sequence that is bound and cleaved by an endonuclease. In the case of a meganuclease, a recognition sequence comprises a pair of inverted, 9 basepair “half sites” which are separated by four basepairs. In the case of a single-chain meganuclease, the N-terminal domain of the protein contacts a first half-site and the C-terminal domain of the protein contacts a second half-site. Cleavage by a meganuclease produces four basepair 3′ “overhangs”. “Overhangs”, or “sticky ends” are short, single-stranded DNA segments that can be produced by endonuclease cleavage of a double-stranded DNA sequence. In the case of meganucleases and single-chain meganucleases derived from I-CreI, the overhang comprises bases 10-13 of the 22 basepair recognition sequence. In the case of a Compact TALEN, the recognition sequence comprises a first CNNNGN sequence that is recognized by the I-TevI domain, followed by a non-specific spacer 4-16 basepairs in length, followed by a second sequence 16-22 bp in length that is recognized by the TAL-effector domain (this sequence typically has a 5′ T base). Cleavage by a Compact TALEN produces two basepair 3′ overhangs. In the case of a CRISPR, the recognition sequence is the sequence, typically 16-24 basepairs, to which the guide RNA binds to direct Cas9 cleavage. Cleavage by a CRISPR produced blunt ends.

As used herein, the term “target site” or “target sequence” refers to a region of the chromosomal DNA of a cell comprising a recognition sequence for a meganuclease.

As used herein, the term “homologous recombination” or “HR” refers to the natural, cellular process in which a double-stranded DNA-break is repaired using a homologous DNA sequence as the repair template (see, e.g. Cahill et al. (2006), Front. Biosci. 11:1958-1976). The homologous DNA sequence may be an endogenous chromosomal sequence or an exogenous nucleic acid that was delivered to the cell.

As used herein, the term “non-homologous end-joining” or “NHEJ” refers to the natural, cellular process in which a double-stranded DNA-break is repaired by the direct joining of two non-homologous DNA segments (see, e.g. Cahill et al. (2006), Front. Biosci. 11:1958-1976). DNA repair by non-homologous end joining is error-prone and frequently results in the untemplated addition or deletion of DNA sequences at the site of repair.

As used herein, the term “re-ligation” refers to a process in which two DNA ends produced by a pair of double-strand DNA breaks are covalently attached to one another with the loss of the intervening DNA sequence but without the gain or loss of any additional DNA sequence. In the case of a pair of DNA breaks that are produced with single-strand overhangs, re-ligation can proceed via annealing of complementary overhangs followed by covalent attachment of 5′ and 3′ ends by a DNA ligase. Re-ligation is distinguished from NHEJ in that it it does not result in the untemplated addition or removal of DNA from the site of repair.

As used herein, unless specifically indicated otherwise, the word “or” is used in the inclusive sense of “and/or” and not the exclusive sense of “either/or.”

### 2.1 Principle of Exon Deletion

The present invention is based, in part, on the hypothesis that certain deletions in the DMD gene that give rise to the Duchenne phenotype can be compensated for by deleting (an) additional exon(s) immediately up- or downstream of the mutation. The DMD-Leiden Database indicates that most of the mutations that cause Duchenne Muscular Dystrophy are deletions of one or more whole exons that cause a shift in reading frame. In many cases, the reading frame can be restored by eliminating the exon immediately before or after the mutation. As shown in Table 1, 29 different Duchenne-causing mutations, representing ˜65% of patients, can be compensated for by deleting a single exon adjacent to the mutation. For example, a patient with disease due to the deletion of DMD Exon 45, which occurs in approximately 7% of patients, can be treated with a therapeutic that deletes Exon 46. Notably, a therapeutic capable of deleting Exon 51 or Exon 45 could be used to treat 15% and 13% of patients, respectively.

TABLE 1

| | | Frequency |
|---|---|---|
| | Additional | in DMD-Leiden |
| Exon(s) deleted in patient | Exon to delete | Database (%) |
| 44, 44-47 | 43 | 5 |
| 35-43, 45, 45-54 | 44 | 8 |
| 18-44, 44, 46-47, 46-48, 46-49, | 45 | 13 |
| 46-51, 46-53 | | |
| 45 | 46 | 7 |
| 51, 51-55 | 50 | 5 |
| 50, 45-50, 48-50, 49-50, 52, | 51 | 15 |
| 52-63 | | |
| 51, 53, 53-55 | 52 | 3 |
| 45-52, 48-52, 49-52, 50-52, | 53 | 9 |
| 52 | | |

### 2.2 Nucleases for Deleting Exons

It is known in the art that it is possible to use a site-specific nuclease to make a DNA break in the genome of a living cell and that such a DNA break can result in permanent modification of the genome via mutagenic NHEJ repair or via HR with a transgenic DNA sequence. The present invention, however, involves co-expression of a pair of nucleases in the same cell. Surprisingly, we found that a pair of nucleases targeted to DNA sites in close proximity to one another (less than 10,000 basepairs apart) can excise the intervening DNA fragment from the genome. Also surprisingly, we found that DNA excision using a pair of nucleases frequently proceeds via a mechanism involving the single-stranded DNA overhangs generated by the nucleases. In experiments involving a pair of meganucleases that generate complementary (i.e. identical) DNA overhangs, it was found that the overhang sequence was frequently conserved following fragment excision and repair of the resulting chromosome ends (see Examples 1 and 2). The mechanism of DNA repair, in this case, appears to direct re-ligation of the broken ends, which has not been observed in mammalian cells. Such precise deletion and re-ligation was not observed when using a pair of meganucleases that generated non-identical overhangs (see Example 3). Thus, in a preferred embodiment, the pair of nucleases used for DMD exon excision are selected to generate complementary overhangs.

To excise an exon efficiently, the pair of nuclease cut sites need to be relatively close together. In general, the closer the two sites are to one another, the more efficient the process will be. Thus, the preferred embodiment of the invention uses a pair of nucleases that cut sequences that are less than 10,000 basepairs or, more preferably, 5,000 basepairs or, still more preferably, less than 2,500 basepairs, or, most preferably, less than 1,500 basepairs apart.

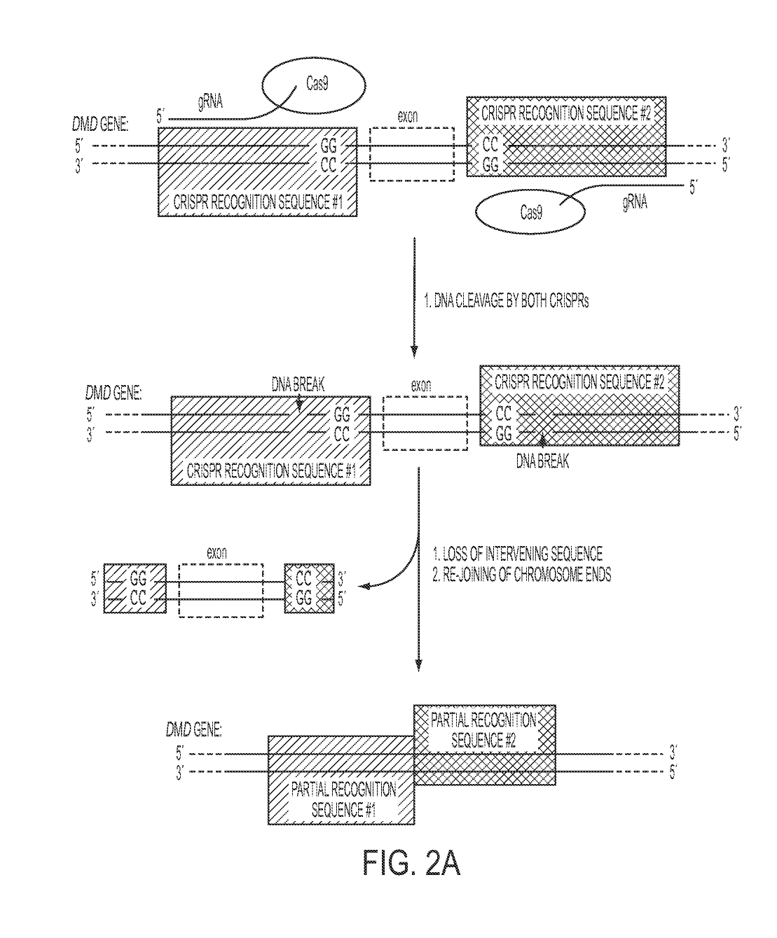

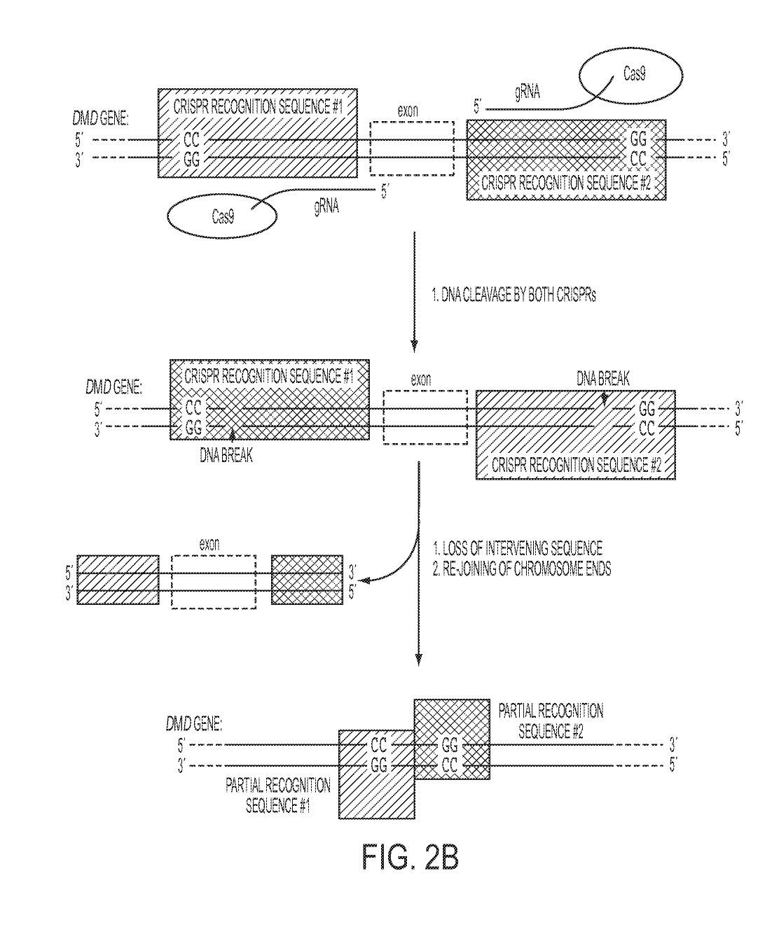

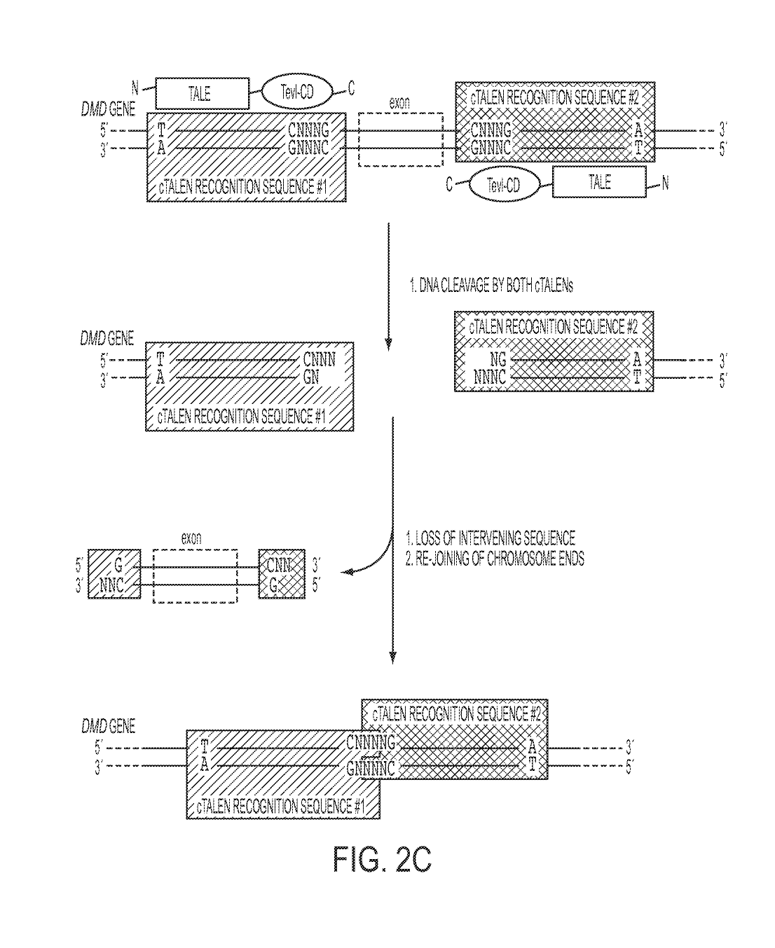

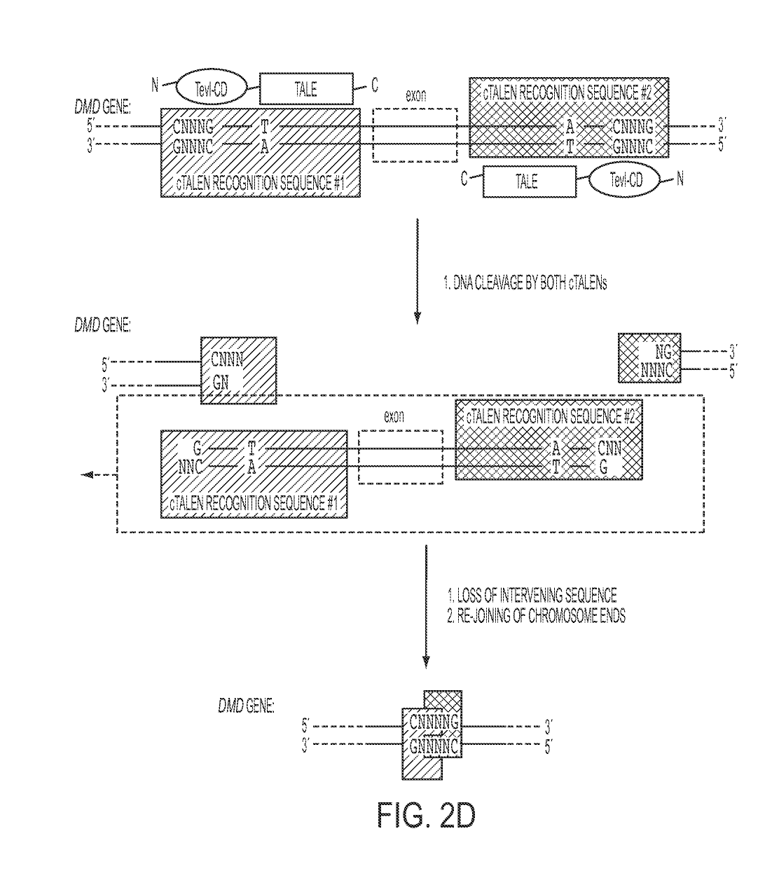

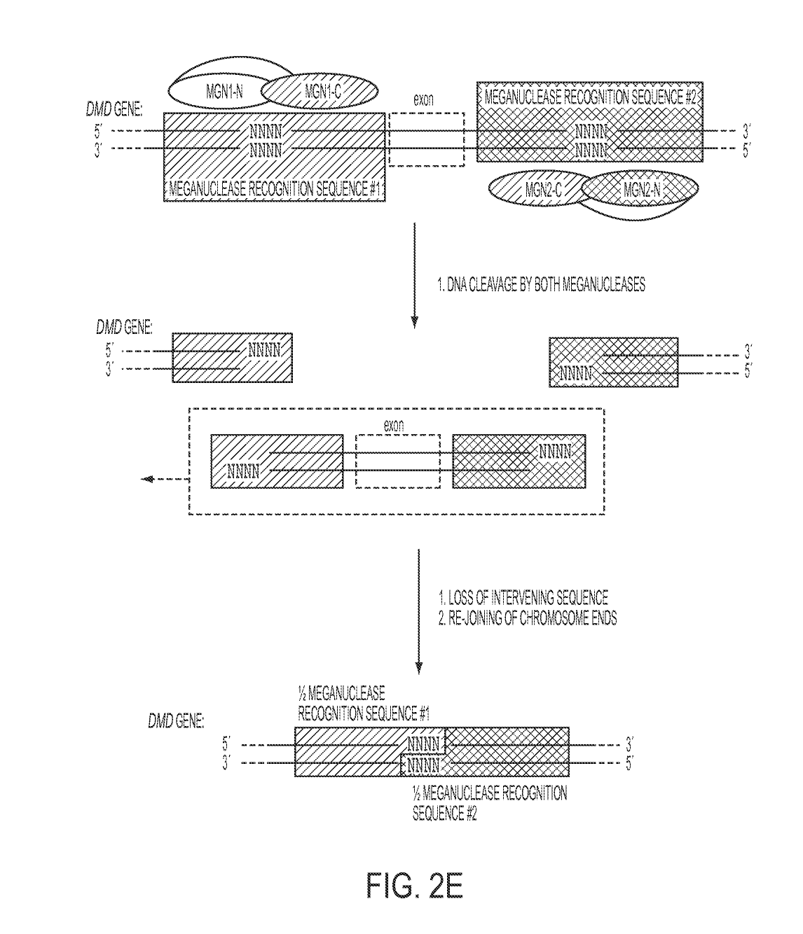

As shown in FIG. 2, a variety of different types of nuclease are useful for practicing the invention. FIGS. 2A and 2B show examples of how the invention can be practiced using a pair of CRISPR nucleases. In this case, the invention can be practiced by delivering three genes to the cell: one gene encoding the Cas9 protein and one gene encoding each of the two guide RNAs. CRISPRs cleave DNA to leave blunt ends which are not generally re-ligated cleanly such that the final product will generally have additional insertion and/or deletion (“indel”) mutations in the sequence. In an alternative embodiment, a “CRISPR Nickase” may be used, as reported in Ran, et al. (2013) Cell. 154:1380-9. To practice this embodiment, it is necessary to express four guide RNAs in the cell, two of which are complementary to the sequence upstream of the exon and two of which are complementary to the sequence downstream of the exon. In this embodiment, the two pairs of guide RNAs hybridize with complementary strands in the target region and each member of the pair produces a single strand DNA nick on one of the strands. The result is a pair of nicks (equivalent to a double-strand break) that can be off-set from one another to yield a single-strand overhang that is advantageous for practicing the invention. Methods for making CRISPRs and CRISPR Nickases that recognize pre-determined DNA sites are known in the art, for example Ran, et al. (2013) Nat Protoc. 8:2281-308.

In alternative embodiments, as diagrammed in FIGS. 2C and 2D, the nuclease pair can be Compact TALENs. A compact TALEN comprises a TAL-effector DNA-binding domain (TALE) fused at its N- or C-terminus to the cleavage domain from I-TevI, comprising at least residues 1-96 and preferably residues 1-182 of I-TevI. The I-TevI cleavage domain recognizes and cuts DNA sequences of the form 5′-CNbNNtG-3′, where “b” represents the site of cleavage of the bottom strand and “t” represents the site of cleavage of the top strand and where“N” is any of the four bases. A Compact TALEN, thus, cleaves to produce two basepair 3′ overhangs. In a preferred embodiment, the Compact TALEN pair used for exon excision is selected to have complementary overhangs that can directly re-ligate. Methods for making TALE domains that bind to pre-determined DNA sites are known in the art, for example Reyon et al. (2012) Nat Biotechnol. 30:460-5.

In the preferred embodiment, as diagrammed in FIG. 2E, the nucleases used to practice the invention are a pair of Single-Chain Meganucleases. A Single-Chain Meganuclease comprises an N-terminal domain and a C-terminal domain joined by a linker peptide. Each of the two domains recognizes half of the Recognition Sequence and the site of DNA cleavage is at the middle of the Recognition Sequence near the interface of the two subunits. DNA strand breaks are offset by four basepairs such that DNA cleavage by a meganuclease generates a pair of four basepair, 3′ single-strand overhangs. In a preferred embodiment, single-chain meganucleases are selected which cut Recognition Sequences with complementary overhangs, as in Examples 1 and 2. Example recognition sequences for DMD Exons 44, 45, and 51 are listed in Tables 2-7. To excise Exon 44, for example, a first meganuclease can be selected which cuts a Recognition Sequence from Table 2, which lists Recognition Sequences upstream of Exon 44. A second meganuclease can then be selected which cuts a Recognition sequences from Table 3, which lists Recognition Sequences downstream of Exon 44. Co-expression of the two meganucleases in the same cell will thus excise Exon 44. Preferably, meganucleases are selected which cut DNA to leave complementary single strand overhangs. For example, SEQ ID NO: 19, if cut by a meganuclease, leaves the overhang sequence: 5′-GTAC-3′. Likewise, SEQ ID NO: 42 if cut by a meganuclease, leaves the overhang sequence: 5′-GTAC-3′. Thus, co-expressing a first meganuclease which cleaves SEQ ID NO: 19 with a second meganuclease which cleaves SEQ ID NO:42 will excise DMD Exon 44 from the genome of a human cell such that complementary overhangs are produced which can be repaired via direct re-ligation.

TABLE 2
Example Meganuclease Recognition Sequences
Upstream of DMD Exon 44

| Recognition Sequence | SEQ ID NO: | Overhang |
|---|---|---|
| TTCTCTGTGGTGAGAAAATTTA | 2 | GTGA |
| TTCACTATTTTGAAATATACAG | 3 | TTGA |
| TATTTTGAAATATACAGCACAA | 4 | ATAT |
| TAACTTTGTTCATATTACTATG | 5 | TCAT |
| ACTTTGTTCATATTACTATGCA | 6 | ATAT |
| CATATTACTATGCAATAGAACA | 7 | ATGC |
| CACTAGAACTTATTACTCCTTT | 8 | TTAT |
| TTTCAGTTGATGAACAGGCAGT | 9 | ATGA |
| AGTTTTGGATCAAGAATAATAT | 10 | TCAA |
| AAAAATATTTTGAAAGGGAATA | 11 | TTGA |
| CCAAATAATTTATTACAATGTT | 12 | TTAT |
| ATCTTTCTTTTAATCAATAAAT | 13 | TTAA |
| TTTTAATCAATAAATATATTCA | 14 | ATAA |
| ACCTTCCATTTAAAATCAGCTT | 15 | TTAA |
| TCAGCTTTTATATTGAGTATTT | 16 | ATAT |
| GCTTTTATATTGAGTATTTTTT | 17 | TTGA |
| TAAAATGTTGTGTGTACATGCT | 18 | GTGT |
| ATGTTGTGTGTACATGCTAGGT | 19 | GTAC |
| GCTAGGTGTGTATATTAATTTT | 20 | GTAT |
| ATTTGTTACTTGAAACTAAACT | 21 | TTGA |
| CTAAACTCTGCAAATGCAGGAA | 22 | GCAA |
| GTGATATCTTTGTCAGTATAAC | 23 | TTGT |
| AAAAAATATACGCTATATCTCT | 24 | ACGC |
| ATCTGTTTTACATAATCCATCT | 25 | ACAT |
| CTGTTTTACATAATCCATCTAT | 26 | ATAA |
| CTATTTTTCTTGATCCATATGC | 27 | TTGA |
| CATATGCTTTTACCTGCAGGCG | 28 | TTAC |

TABLE 3
Example Meganuclease Recognition Sequences
Downstream of DMD Exon 44

| Recognition Sequence | SEQ ID NO: | Overhang |
|---|---|---|
| AAATTACTTTTGACTGTTGTTG | 29 | TTGA |
| TGACTGTTGTTGTCATCATTAT | 30 | TTGT |
| TTGTTGTCATCATTATATTACT | 31 | TCAT |
| TTGTCATCATTATATTACTAGA | 32 | TTAT |
| ATCATTATATTACTAGAAAGAA | 33 | TTAC |
| AAAATTATCATAATGATAATAT | 34 | ATAA |
| ATGGACTTTTTGTGTCAGGATG | 35 | TTGT |
| GGACTTTTTGTGTCAGGATGAG | 36 | GTGT |
| GGAGCTGGTTTATCTGATAAAC | 37 | TTAT |
| ATTGAATCTGTGACAGAGGGAA | 38 | GTGA |
| AGGGAAGCATCGTAACAGCAAG | 39 | TCGT |
| GGGCAGTGTGTATTTCGGCTTT | 40 | GTAT |
| TATATTCTATTGACAAAATGCC | 41 | TTGA |
| TAATTGTTGGTACTTATTGACA | 42 | GTAC |
| TGTTGGTACTTATTGACATTTT | 43 | TTAT |
| TTTTATGGTTTATGTTAATAGG | 44 | TTAT |

TABLE 4
Example Meganuclease Recognition Sequences
Upstream of DMD Exon 45

| Recognition Sequence | SEQ ID NO: | Overhang |
|---|---|---|
| AGTTTTTTTTTAATACTGTGAC | 45 | TTAA |
| TTTAATACTGTGACTAACCTAT | 46 | GTGA |
| TTTCACCTCTCGTATCCACGAT | 47 | TCGT |
| TCACCTCTCGTATCCACGATCA | 48 | GTAT |
| CTCGTATCCACGATCACTAAGA | 49 | ACGA |
| CCAAATACTTTGTTCATGTTTA | 50 | TTGT |
| GGAACATCCTTGTGGGGACAAG | 51 | TTGT |
| AATTTGCTCTTGAAAAGGTTTC | 52 | TTGA |
| CTAATTGATTTGTAGGACATTA | 53 | TTGT |
| TTCCCTGACACATAAAAGGTGT | 54 | ACAT |
| CCCTGACACATAAAAGGTGTCT | 55 | ATAA |
| CTTTCTGTCTTGTATCCTTTGG | 56 | TTGT |
| ATCCTTTGGATATGGGCATGTC | 57 | ATAT |
| TGGATATGGGCATGTCAGTTTC | 58 | GCAT |
| GATATGGGCATGTCAGTTTCAT | 59 | ATGT |
| GAAATTTTCACATGGAGCTTTT | 60 | ACAT |
| TTTCTTTCTTTGCCAGTACAAC | 61 | TTGC |
| TCTTTGCCAGTACAACTGCATG | 62 | GTAC |
| TTTGGTATCTTACAGGAACTCC | 63 | TTAC |

TABLE 5
Example Meganuclease Recognition Sequences
Downstream of DMD Exon 45

| Recognition Sequence | SEQ ID NO: | Overhang |
|---|---|---|
| AAGAATATTTCATGAGAGATTA | 64 | TCAT |
| GAATATTTCATGAGAGATTATA | 65 | ATGA |
| TGAGAGATTATAAGCAGGGTGA | 66 | ATAA |
| AAGGCACTAACATTAAAGAACC | 67 | ACAT |
| TCAACAGCAGTAAAGAAATTTT | 68 | GTAA |
| TTCTTTTTTTCATATACTAAAA | 69 | TCAT |
| CTAAAATATATACTTGTGGCTA | 70 | ATAC |
| TGAATATCTTCAATATATTTTA | 71 | TCAA |
| CAATTATAAATGATTGTTTTGT | 72 | ATGA |
| ATGATTGTTTTGTAGGAAAGAC | 73 | TTGT |
| TCATATTTTGTACAAAATAAAC | 74 | GTAC |

TABLE 6
Example Meganuclease Recognition Sequences
Upstream of DMD Exon 51

| Recognition Sequence | SEQ ID NO: | Overhang |
|---|---|---|
| ATACGTGTATTGCTTGTACTAC | 75 | TTGC |
| GTATTGCTTGTACTACTCACTG | 76 | GTAC |
| ACTGAATCTACACAACTGCCCT | 77 | ACAC |
| TGAATCTACACAACTGCCCTTA | 78 | ACAA |
| CAACTGCCCTTATGACATTTAC | 79 | TTAT |
| GGTAAATACATGAAAAATGCTT | 80 | ATGA |
| TTGCCTTGCTTACTGCTTATTG | 81 | TTAC |
| GCTTACTGCTTATTGCTAGTAC | 82 | TTAT |
| TAGTACTGAACAAATGTTAGAA | 83 | ACAA |
| ACTGAACAAATGTTAGAACTGA | 84 | ATGT |
| AAGATTTATTTAATGACTTTGA | 85 | TTAA |
| CAGTATTTCATGTCTAAATAGA | 86 | ATGT |
| GGTTTTTCTTCACTGCTGGCCA | 87 | TCAC |
| CAATCTGAAATAAAAAGAAAAA | 88 | ATAA |
| CTGCTCCCAGTATAAAATACAG | 89 | GTAT |
| AAGAACGTTTCATTGGCTTTGA | 90 | TCAT |
| ACTTCCTATTCAAGGGAATTTT | 91 | TCAA |
| TGTTTTTTCTTGAATAAAAAAA | 92 | TTGA |
| TTTTCTTGAATAAAAAAAAAAT | 93 | ATAA |
| TTGTTTTCTTTACCACTTCCAC | 94 | TTAC |
| ACAATGTATATGATTGTTACTG | 95 | ATGA |
| TGTATATGATTGTTACTGAGAA | 96 | TTGT |
| CTTGTCCAGGCATGAGAATGAG | 97 | GCAT |
| TGTCCAGGCATGAGAATGAGCA | 98 | ATGA |
| AATCGTTTTTTAAAAAATTGTT | 99 | TTAA |
| TTCTACCATGTATTGCTAAACA | 100 | GTAT |
| TACCATGTATTGCTAAACAAAG | 101 | TTGC |
| TATAATGTCATGAATAAGAGTT | 102 | ATGA |
| ATGTCATGAATAAGAGTTTGGC | 103 | ATAA |
| TTTTCCTTTTTGCAAAAACCCA | 104 | TTGC |
| TTCCTTTTTGCAAAAACCCAAA | 105 | GCAA |

TABLE 7
Example Meganuclease Recognition Sequences
Downstream of DMD Exon 51

| Recognition Sequence | SEQ ID NO: | Overhang |
|---|---|---|
| AGTTCTTAGGCAACTGTTTCTC | 106 | GCAA |
| TCTCTCTCAGCAAACACATTAC | 107 | GCAA |
| TAAGTATAATCAAGGATATAAA | 108 | TCAA |
| AGTAGCCATACATTAAAAAGGA | 109 | ACAT |
| AGGAATATACAAAAAAAAAAAA | 110 | ACAA |
| AGAAACCTTACAAGAATAGTTG | 111 | ACAA |
| CAAGAATAGTTGTCTCAGTTAA | 112 | TTGT |
| ATCTATTTTATACCAAATAAGT | 113 | ATAC |
| TTATACCAAATAAGTCACTCAA | 114 | ATAA |
| TTTGTTTTGGCACTACGCAGCC | 115 | GCAC |
| TAAGGATAATTGAAAGAGAGCT | 116 | TTGA |
| AGAAAAGTAACAAAACATAAGA | 117 | ACAA |
| TTAAAGTTGGCATTTATGCAAT | 118 | GCAT |
| AGTTGGCATTTATGCAATGCCA | 119 | TTAT |
| AACATGTTTTTAATACAAATAG | 120 | TTAA |
| TACATTGATGTAAATATGGTTT | 121 | GTAA |
| ATATCTTTTATATTTGTGAATG | 122 | ATAT |
| CTTTTATATTTGTGAATGATTA | 123 | TTGT |
| TGTGAATGATTAAGAAAAATAA | 124 | TTAA |
| AATTGTTATACATTAAAGTTTT | 125 | ACAT |
| AAAGTTTTTTCACTTGTAACAG | 126 | TCAC |
| TAACAGCTTTCAAGCCTTTCTA | 127 | TCAA |
| GGTATTTAGGTATTAAAGTACT | 128 | GTAT |
| TACTACCTTTTGAAAAAACAAG | 129 | TTGA |
| GGAATTTCTTTGTAAAATAAAC | 130 | TTGT |
| AACCTGCATTTAAAGGCCTTGA | 131 | TTAA |
| TGAGCTTGAATACAGAAGACCT | 132 | ATAC |
| TGATTGTGGTCAAGCCATCTCT | 133 | TCAA |
| CTATTCTGAGTACAGAGCATAC | 134 | GTAC |

### 2.3 Methods for Delivering and Expressing Nucleases

Treating Duchenne Muscular Dystrophy using the invention requires that a pair of nucleases be expressed in a muscle cell. The nucleases can be delivered as purified protein or as RNA or DNA encoding the nucleases. In one embodiment, the nuclease proteins or mRNA or vector encoding the nucleases are supplied to muscle cells via intramuscular injection (Maltzahn, et al. (2012) Proc Natl Acad Sci USA. 109:20614-9) or hydrodynamic injection (Taniyama et al. (2012) Curr Top Med Chem. 12:1630-7; Hegge, et al. (2010) Hum Gene Ther. 21:829-42). To facilitate cellular uptake, the proteins or nucleic acid(s) can be coupled to a cell penetrating peptide to facilitate uptake by muscle cells. Examples of cell pentrating peptides known in the art include poly-arginine (Jearawiriyapaisarn, et al. (2008) Mol Ther. 16:1624-9), TAT peptide from the HIV virus (Hudecz et al. (2005), Med. Res. Rev. 25: 679-736), MPG (Simeoni, et al. (2003) Nucleic Acids Res. 31:2717-2724), Pep-1 (Deshayes et al. (2004) Biochemistry 43: 7698-7706, and HSV-1 VP-22 (Deshayes et al. (2005) Cell Mol Life Sci. 62:1839-49. Alternatively, cell penetration can be facilitated by liposome encapsulation (see, e.g., Lipofectamine™, Life Technologies Corp., Carlsbad, Calif.). The liposome formulation can be used to facilitate lipid bilayer fusion with a target cell, thereby allowing the contents of the liposome or proteins associated with its surface to be brought into the cell.

In some embodiments, the genes encoding a pair of nucleases are delivered using a viral vector. Such vectors are known in the art and include lentiviral vectors, adenoviral vectors, and adeno-associated virus (AAV) vectors (reviewed in Vannucci, et al. (2013 New Microbiol. 36:1-22). In some embodiments, the viral vectors are injected directly into muscle tissue. In alternative embodiments, the viral vectors are delivered systemically. Example 3 describes a preferred embodiment in which the muscle is injected with a recombinant AAV virus encoding a pair of single-chain meganucleases. It is known in the art that different AAV vectors tend to localize to different tissues. Muscle-tropic AAV serotypes include AAV1, AAV9, and AAV2.5 (Bowles, et al. (2012) Mol Ther. 20:443-55). Thuse, these serotypes are preferred for the delivery of nucleases to muscle tissue.

If the nuclease genes are delivered in DNA form (e.g. plasmid) and/or via a viral vector (e.g. AAV) they must be operably linked to a promoter. In some embodiments, this can be a viral promoter such as endogenous promoters from the viral vector (e.g. the LTR of a lentiviral vector) or the well-known cytomegalovirus- or SV40 virus-early promoters. In a preferred embodiment, the nuclease genes are operably linked to a promoter that drives gene expression preferentially in muscle cells. Examples of muscle-specific promoters include C5-12 (Liu, et al. (2004) Hum Gene Ther. 15:783-92), the muscle-specific creatine kinase (MCK) promoter (Yuasa, et al. (2002) Gene Ther. 9:1576-88), or the smooth muscle 22 (SM22) promoter (Haase, et al. (2013) BMC Biotechnol. 13:49-54). In some embodiments, the nuclease genes are under the control of two separate promoters. In alternative embodiments, the genes are under the control of a single promoter and are separated by an internal-ribosome entry site (IRES) or a 2 A peptide sequence (Szymczak and Vignali (2005) Expert Opin Biol Ther. 5:627-38).

It is envisioned that a single treatment will permanently delete exons from a percentage of patient cells. In preferred embodiments, these cells will be myoblasts or other muscle precurser cells that are capable of replicating and giving rise to whole muscle fibers that express functional (or semi-functional) dystrophin. If the frequency of exon deletion is low, however, it may be necessary to perform multiple treatments on each patient such as multiple rounds of intramuscular injections.

### EXAMPLES

This invention is further illustrated by the following examples, which should not be construed as limiting. Those skilled in the art will recognize, or be able to ascertain, using no more than routine experimentation, numerous equivalents to the specific substances and procedures described herein. Such equivalents are intended to be encompassed in the scope of the claims that follow the examples below.

### Example 1

### Deletion of DMD Exon 44 Using a Pair of Engineered, Single-Chain Meganucleases

1. Meganucleases that Recognize SEQ ID NO: 19 and SEQ ID NO: 42

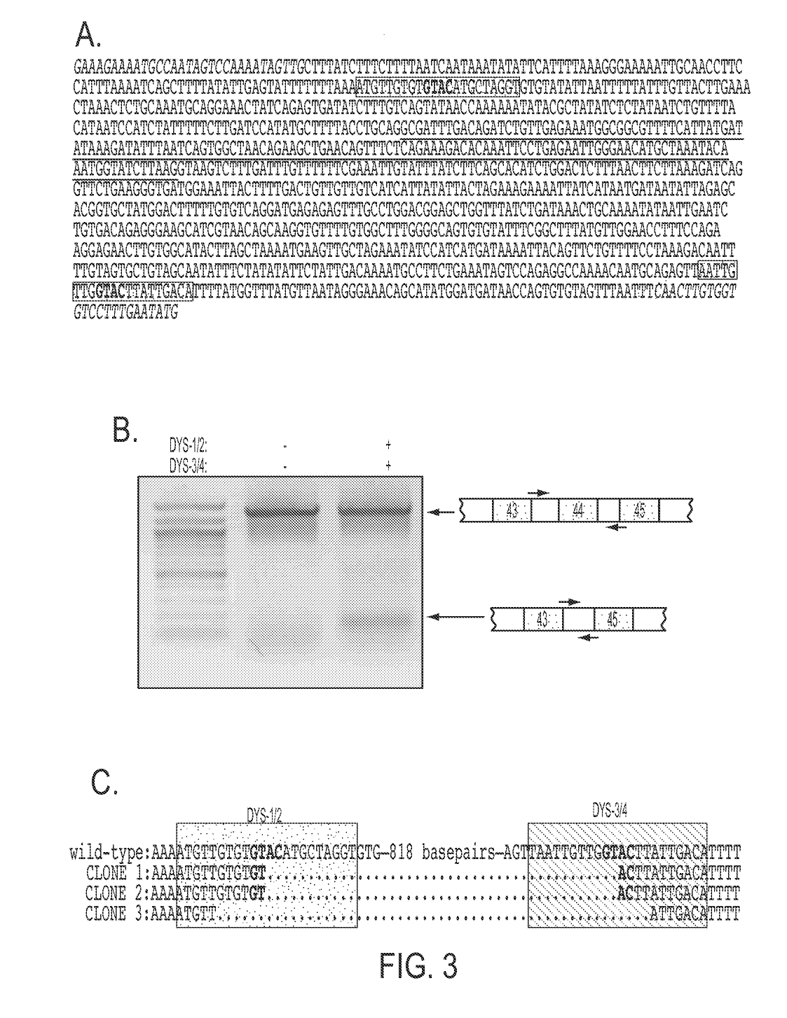

An engineered meganuclease (SEQ ID NO: 135) was produced which recognizes and cleaves SEQ ID NO: 19. This meganuclease is called “DYS-1/2”. A second engineered meganuclease (SEQ ID NO: 136) was produced which recognizes and cleaves SEQ ID NO: 42. This meganuclease is called “DYS-3/4” (FIG. 3A). Each meganuclease comprises an N-terminal nuclease-localization signal derived from SV40, a first meganuclease subunit, a linker sequence, and a second meganuclease subunit.

### 2. Deletion of DMD Exon 44 in HEK-293 Cells

Human embryonic kidney (HEK-293) cells were co-transfected with mRNA encoding DYS-1/2 and DYS-3/4. mRNA was prepared by first producing a PCR template for an in vitro transcription reaction (SEQ ID NO: 139 and SEQ ID NO: 140. Each PCR product included a T7 promoter and 609 bp of vector sequence downstream of the meganuclease gene. The PCR product was gel purified to ensure a single template. Capped (m7G) RNA was generated using the RiboMAX T7 kit (Promega) according to the manufacturer's instructions and. Ribo m7G cap analog (Promega) was included in the reaction and 0.5 ug of the purified meganuclease PCR product served as the DNA template. Capped RNA was purified using the SV Total RNA Isolation System (Promega) according to the manufacturer's instructions.

1.5×10⁶ HEK-293 cells were nucleofected with 1.5×10¹² copies of DYS-1/2 mRNA and 1.5×10¹² copies of DYS-3/4 mRNA (2×10⁶ copies/cell) using an Amaxa Nucleofector II device (Lonza) according to the manufacturer's instructions. 48 hours post-transfection, genomic DNA was isolated from the cells using a FlexiGene kit (Qiagen) according to the manufacturer's instructions. The genomic DNA was then subjected to PCR using primers flanking the DYS-1/2 and DYS-3/4 cut sites (SEQ ID NO: 141 and SEQ ID NO:142). When PCR products were resolved by agarose gel electrophoresis, it was apparent that cells co-expressing DYS-1/2 and DYS-3/4 yielded two PCR products with apparent lengths of 1079 basepairs and 233 basepairs whereas genomic DNA from untransfected HEK-293 cells yielded only the larger product (FIG. 3B). The larger product is consistent with the expected size of a PCR fragment from cells with intact DMD Exon 44. The smaller product is consistent with the expected size of a PCR fragment from cells in which Exon 44 has been excised from the DMD gene.

The smaller PCR product was isolated from the gel and cloned into a bacterial plasmid (pUC-19) for sequence analysis. Three plasmid clones were sequenced, all of which were found to have Exon 44 deleted (FIG. 3C). Surprisingly, two of the three plasmids carried PCR products from cells in which the deletion consisted precisely of the region intervening the expected DYS-1/2 and DYS-3/4-induced DNA breaks. It appears that the two meganucleases cleaved their intended recognition sites, leaving compatible 5′-GTAC-3′ overhangs, the intervening fragment comprising Exon 44 was lost, and the two chromosome ends were then re-ligated. The third plasmid clone carried a PCR product from a cell in which the region intervening the two cleavage sites was excised along with 10 additional bases.

### 3. Conclusions

We have demonstrated that it is possible to use a pair of engineered single-chain meganucleases to excise a fragment from the human genome in a cultured cell line. The DNA removal and repair process appears to have proceeded via a mechanism that involves the 3′ overhangs produced by the nucleases, suggesting that the process is more efficient when the overhangs are complementary and able to anneal to one another.

### Example 2

### Deletion of DMD Exon 45 Using a Pair of Engineered, Single-Chain Meganucleases

1. Meganucleases that Recognize SEQ ID NO: 62 and SEQ ID NO: 74

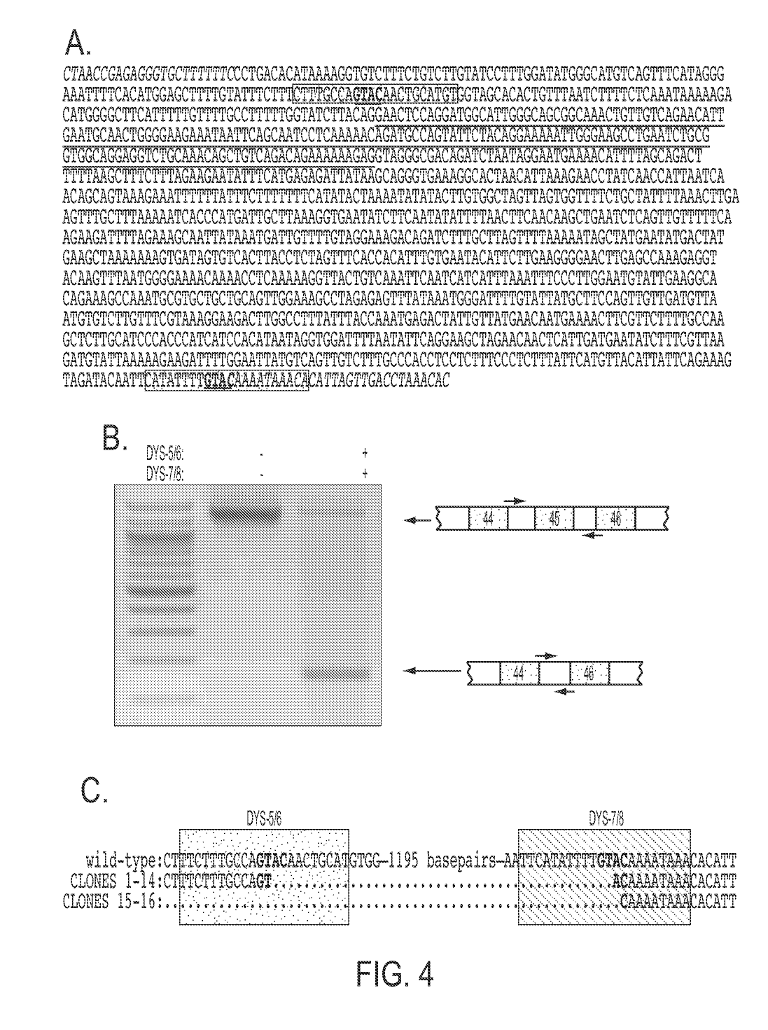

An engineered meganuclease (SEQ ID NO: 137) was produced which recognizes and cleaves SEQ ID NO: 62. This meganuclease is called “DYS-5/6”. A second engineered meganuclease (SEQ ID NO: 138) was produced which recognizes and cleaves SEQ ID NO: 74. This meganuclease is called “DYS-7/8” (FIG. 4A). Each meganuclease comprises an N-terminal nuclease-localization signal derived from SV40, a first meganuclease subunit, a linker sequence, and a second meganuclease subunit.

### 2. Deletion of DMD Exon 45 in HEK-293 Cells

Human embryonic kidney (HEK-293) cells were co-transfected with mRNA encoding DYS-5/6 and DYS-7/8. mRNA was prepared by first producing a PCR template for an in vitro transcription reaction (SEQ ID NO: 143(20) and SEQ ID NO: 144(21). Each PCR product included a T7 promoter and 609 bp of vector sequence downstream of the meganuclease gene. The PCR product was gel purified to ensure a single template. Capped (m7G) RNA was generated using the RiboMAX T7 kit (Promega) according to the manufacturer's instructions and. Ribo m7G cap analog (Promega) was included in the reaction and 0.5 ug of the purified meganuclease PCR product served as the DNA template. Capped RNA was purified using the SV Total RNA Isolation System (Promega) according to the manufacturer's instructions.

1.5×10⁶ HEK-293 cells were nucleofected with 1.5×10¹² copies of DYS-5/6 mRNA and 1.5×10¹² copies of DYS-7/8 mRNA (2×10⁶ copies/cell) using an Amaxa Nucleofector II device (Lonza) according to the manufacturer's instructions. 48 hours post-transfection, genomic DNA was isolated from the cells using a FlexiGene kit (Qiagen) according to the manufacturer's instructions. The genomic DNA was then subjected to PCR using primers flanking the DYS-5/6 and DYS-7/8 cut sites (SEQ ID NO: 145 and SEQ ID NO:146). When PCR products were resolved by agarose gel electrophoresis, it was apparent that cells co-expressing DYS-5/6 and DYS-7/8 yielded two PCR products with apparent lengths of 1384 basepairs and 161 basepairs whereas genomic DNA from untransfected HEK-293 cells yielded only the larger product (FIG. 4B). The larger product is consistent with the expected size of a PCR fragment from cells with intact DMD Exon 45. The smaller product is consistent with the expected size of a PCR fragment from cells in which Exon 45 has been excised from the DMD gene.

The smaller PCR product was isolated from the gel and cloned into a bacterial plasmid (pUC-19) for sequence analysis. 16 plasmid clones were sequenced, all of which were found to have Exon 45 deleted (FIG. 4C). Surprisingly, 14 of the 16 plasmids carried PCR products from cells in which the deletion consisted precisely of the region intervening the expected DYS-5/6 and DYS-7/8-induced DNA breaks. It appears that the two meganucleases cleaved their intended recognition sites, leaving compatible 5′-GTAC-3′ overhangs, the intervening fragment comprising Exon 45 was lost, and the two chromosome ends were then re-ligated. The two remaining plasmid clones carried PCR product from cells in which the region intervening the two cleavage sites was excised along with 36 additional bases.

### 3. Conclusions

We have demonstrated that it is possible to use a pair of engineered single-chain meganucleases to excise a fragment from the human genome in a cultured cell line. The DNA removal and repair process appears to have proceeded via a mechanism that involves the 3′ overhangs produced by the nucleases, suggesting that the process is more efficient when the overhangs are complementary and able to anneal to one another.

### Example 3

### Deletion of DMD Exon 23 in a Mouse Using AAV-Delivered Meganucleases

### 1. Development of Nucleases to Delete Mouse DMD Exon 23

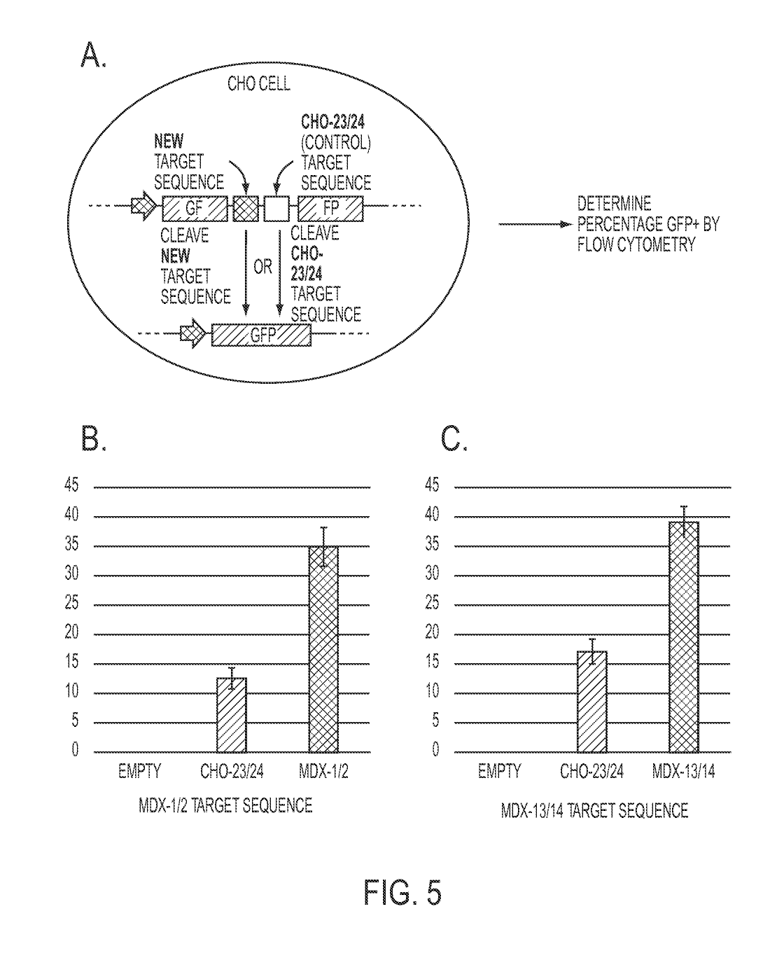

The standard mouse model of DMD is the mdx mouse, which has a point mutation in Exon 23 that introduces a premature stop codon (Sicinski et al. (1989) Science. 244:1578-80). In the mouse, DMD Exon 23 is 213 basepairs, equivalent to 71 amino acids. Thus, we reasoned that it should be possible to delete Exon 23 in its entirety and thereby remove the stop codon while maintaining the reading frame of the DMD gene. To this end, we developed a pair of single-chain meganucleases called “MDX-1/2” (SEQ ID NO: 147) and “MDX-13/14” (SEQ ID NO: 148). The former recognizes a DNA sequence upstream of mouse DMD Exon 23 (SEQ ID NO: 149) while the latter recognizes a DNA sequence downstream of mouse DMD Exon 23 (SEQ ID NO: 150). The nucleases were tested, initially, using a reporter assay called “iGFFP” in CHO cells as shown in FIG. 5. Both nucleases were found to efficiently cut their intended DNA sites using this assay.

### 2. Deletion of Mouse DMD Exon 23 in Mouse Myoblast Cells

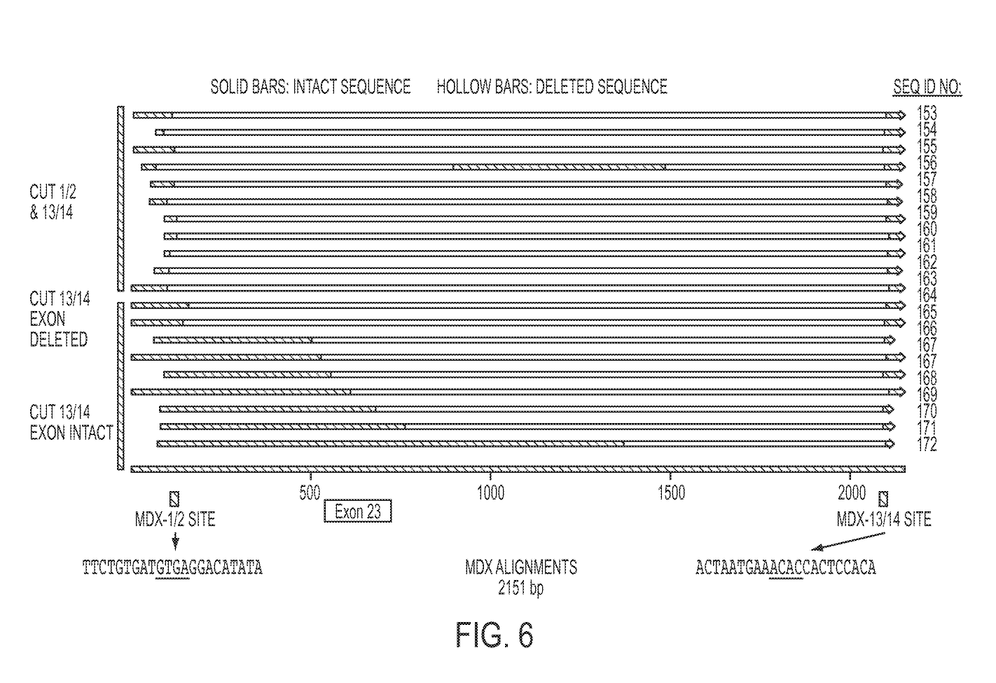

A mouse myoblast cell line (C2C12) was co-transfected with in vitro transcribed mRNA encoding the MDX-1/2 and MDX-13/14 nucleases. mRNA was produced using the RiboMAX T7 kit from Promega. 1e6 C2C12 cells were Nucleofected with a total of 2e6 copies/cell of mRNA encoding each MDX enzyme pairs (1 e6 copies of each mRNA) using an Amaxa 2b device and the B-032 program. After 96 hours, cells were cloned by limiting dilution in 96-well plates. After approximately 2 weeks growth, cells were harvested and genomic DNA was isolated using a FlexiGene kit from Qiagen. A PCR product was then generated for each clone using a forward primer in DMD Intron 22 (SEQ ID NO: 151) and a reverse primer in Intron 23 (SEQ ID NO: 152). 60 of the PCR products were then cloned and sequenced. 20 of the sequences had deletions consistent with meganuclease-induced cleavage of the DMD gene followed by mutagenic DNA repair (FIG. 6, SEQ ID NO:153-172). 11 of the sequences were missing at least a portion of the MDX-1/2 and MDX-13/14 recognition sites, as well as Exon 23 (SEQ ID NO:153-163). These sequences were likely derived from cells in which both nucleases cut their intended sites and the intervening sequence was deleted. 4 of the sequences were missing Exon 23 but had an intact MDX-1/2 recognition sequence (SEQ ID NO:164-167). These appear to be due to DNA cleavage by MDX-13/14 alone followed by the deletion of a large amount of sequence. Five of the sequences had an intact MDX-1/2 recognition site and all or a portion of Exon 23 but were missing all or a portion of the MDX-13/14 recognition site (SEQ ID NO:168-172). These sequences appear to be due to DNA cleavage by MDX-13/14 alone followed by the deletion of a smaller amount of sequence insufficient to eliminate all of Exon 23. In stark contrast to the experiments in Examples 1 and 2, we did not obtain a consistent DNA sequence following the deletion of DMD Exon 23 in the mouse cells. This is likely because the two MDX meganucleases do not generate DNA breaks with compatible 3′ overhangs. MDX-1/2 generates an overhang with the sequence 5′-GTGA-3′ and MDX-13/14 generates an overhang with the sequence 5′-ACAC-3′. Thus, we conclude that the consistent sequence results obtained in Examples 1 and 2 are due to the compatibility of the 3′ overhangs generated by the pair of meganucleases.

### 3. Generation of Recombinant AAV Vectors for Delivery of a Pair of Engineered Nucleases.

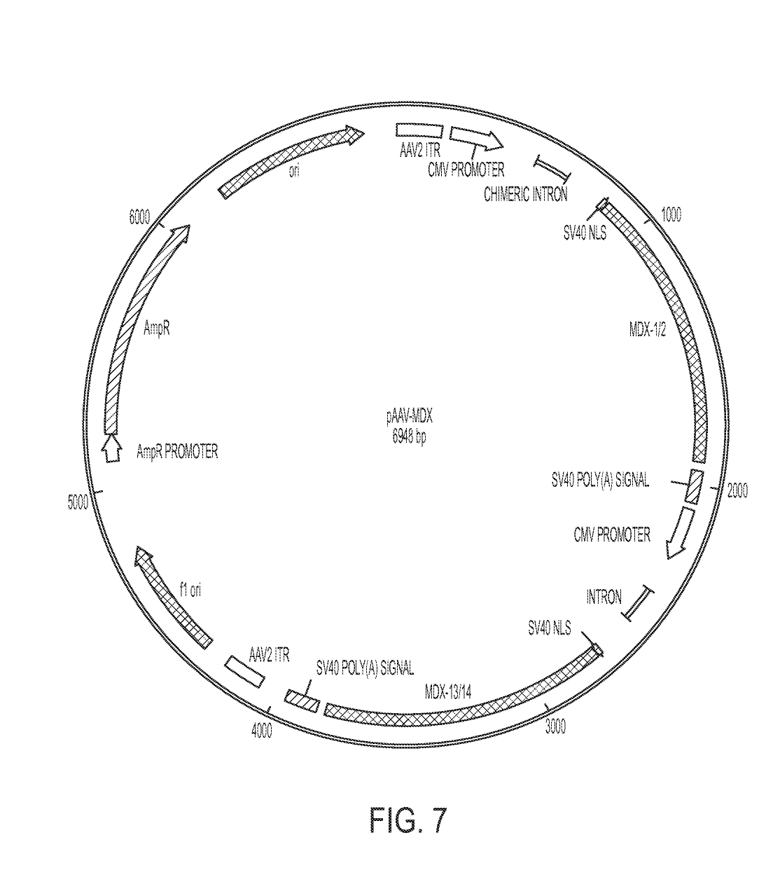

To produce AAV vectors for simultaneous delivery of MDX-1/2 and MDX-13/14 genes, we first produced a “packaging” plasmid called “pAAV-MDX” (FIG. 7, SEQ ID NO. 173) comprising a pair of inverted terminal repeat (ITR) sequences from AAV2, as well as the gene coding sequences for the MDX-1/2 and MDX-13/14 meganucleases, each under the control of a CMV Early promoter. This vector was used to produce recombinant AAV2 virus by co-transfection of HEK-293 cells with an Ad helper plasmid according to the method of Xiao, et at (Xiao, et al. (1998) J. Virology 72:2224-2232). Virus was then isolated by cesium-chloride gradient centrifugation as described by Grieger and Samulski (Grieger and Samulski (2012) Methods Enzymol. 507:229-254). To confirm that the resulting virus particles were infectious and capable of expressing both engineered meganucleases, they were added to cultured iGFFP CHO cells carrying reporter cassettes for either MDX-1/2 or MDX-13/14 (see FIG. 5A). The addition of recombinant virus particles to the CHO line carrying a reporter cassette for MDX-1/2 resulted in GFP gene expression in 7.1% of the cells. The addition of virus to the CHO line carrying a reporter for MDX-13/14 resulted in GFP gene expression in 10.2% of cells. Thus, we conclude that the virus was able to transduce CHO cells and that transduced cells expressed both nucleases.

### 4. Deletion of DMD Exon 23 in Mouse Muscle Following AAV Delivery of a Pair of Meganuclease Genes.

Recombinant AAV1 virus particles carrying the MDX-1/2 and MDX-13/14 genes were produced as described above. Three hindlimb TA muscles from a pair of mdx mice were injected with virus as described in Xiao, et at (Xiao, et al. (1998) J. Virology 72:2224-2232). One muscle from one mouse was not injected as a negative control. Muscles from the two mice were harvested at 4 days or 7 days post-injection and genomic DNA was isolated from the muscle tissue. The genomic region surrounding DMD Exon 23 was amplified by PCR using a first primer pair (SEQ ID NO:151 and SEQ ID NO: 152). This reaction was then used to template a second PCR reaction using a “nested” primer pair (SEQ ID NO:174 and SEQ ID NO: 175) to eliminate non-specific PCR products. PCR products were then visualized on an agarose gel and it was found that genomic DNA from the three AAV1 injected muscles, but not the un-injected control muscle, yielded smaller PCR products that were consistent in size with the product expected following deletion of DMD Exon 23 by the MDX-1/2 and MDX-13/14 meganucleases. The smaller PCR products were then cloned and sequenced. Three unique sequences were obtained, each of which comprised a portion of the mouse DMD gene including part of Intron 22 and Intron 23 but lacking Exon 23 and all of the sequence intervening the cut sites for the MDX-1/2 and MDX-13/14 meganucleases (SEQ ID NO: 176-178). Thus, we have demonstrated that a pair of meganucleases delivered by AAV can be used to delete a portion of the DMD gene in vivo from mouse muscle.

### 5. Conclusions

We have demonstrated that the genes encoding a pair of engineered single-chain meganucleases can be delivered to cells and organisms using recombinant AAV vectors and that meganucleases so delivered are able to cleave genomic DNA in the cell and delete fragments of DNA from the genome. We have further demonstrated that a pair of meganuclease-induced DNA breaks that do not generate compatible overhangs will not re-ligate to yield a defined sequence outcome following removal of the intervening sequence. Thus, for therapeutic applications in which a defined sequence outcome is desirable, it is preferable to use a pair of nucleases that generate identical overhangs.

SEQUENCE LISTING

SEQ ID NO: 1 (wild-type I-CreI, Genbank Accession # PO5725)

| | |
|---|---|
| 1 | MNTKYNKEFL LYLAGFVDGD GSIIAQIKPN QSYKFKHQLS LAFQVTQKTQ RRWFLDKLVD |
| 61 | EIGVGYVRDR GSVSDYILSE IKPLHNFLTQ LQPFLKLKQK QANLVLKIIW RLPSAKESPD |
| 121 | KFLEVCTWVD QIAALNDSKT RKTTSETVRA VLDSLSEKKK SSP |

SEQ ID NO: 135 (DYS-1/2)

| | |
|---|---|
| 1 | MAPKKKRKVH MNTKYNKEFL LYLAGFVDGD GSIYAWISPS QTCKFKHRLM LRFIVSQKTQ |
| 61 | RRWFLDKLVD EIGVGYVQDC GSVSEYRLSE IKPLHNFLTQ LQPFLKLKQK QANLVLKIIE |
| 121 | QLPSAKESPD KFLEVCTWVD QIAALNDSKT RKTTSETVRA VLDSLPGSVG GLSPSQASSA |
| 181 | ASSASSSPGS GISEALRAGA GSGTGYNKEF LLYLAGFVDG DGSIYACILP TQRQKFKHGL |
| 241 | TLYFRVTQKT QRRWFLDKLV DEIGVGYVLD FGSVSCYSLS QIKPLHNFLT QLQPFLKLKQ |
| 301 | KQANLVLKII EQLPSAKESP DKFLEVCTWV DQIAALNDSK TRKTTSETVR AVLDSLSEKK |
| 361 | KSSP |

SEQ ID NO: 136 (DYS-3/4)

| | |
|---|---|
| 1 | MAPKKKRKVH MNTKYNKEFL LYLAGFVDGD GSIFASIRPR QTSKFKHALA LFFVVGQKTQ |
| 61 | RRWFLDKLVD EIGVGYVYDR GSVSVYQLSQ IKPLHNFLTQ LQPFLKLKQK QANLVLKIIE |
| 121 | QLPSAKESPD KFLEVCTWVD QIAALNDSKT RKTTSETVRA VLDSLPGSVG GLSPSQASSA |
| 181 | ASSASSSPGS GISEALRAGA GSGTGYNKEF LLYLAGFVDG DGSIIACIRP HQAYKFKHQL |
| 241 | CLSFCVYQKT QRRWFLDKLV DEIGVGYVTD AGSVSSYRLS EIKPLHNFLT QLQPFLKLKQ |
| 301 | KQANLVLKII EQLPSAKESP DKFLEVCTWV DQIAALNDSK TRKTTSETVR AVLDSLSEKK |
| 361 | KSSP |

SEQ ID NO: 137 (DYS-5/6)

| | |
|---|---|
| 1 | MAPKKKRKVH MNTKYNKEFL LYLAGFVDGD GSIFACIQPD QRAKFKHTLR LSFEVGQKTQ |
| 61 | RRWFLDKLVD EIGVGYVNDS GSVSKYRLSQ IKPLHNFLTQ LQPFLKLKQK QANLVLKIIE |
| 121 | QLPSAKESPD KFLEVCTWVD QIAALNDSKT RKTTSETVRA VLDSLPGSVG GLSPSQASSA |
| 181 | ASSASSSPGS GISEALRAGA GSGTGYNKEF LLYLAGFVDG DGSIYATIQP TQCAKFKHQL |
| 241 | TLRFSVSQKT QRRWFLDKLV DEIGVGYVCD KGSVSEYMLS EIKPLHNFLT QLQPFLKLKQ |
| 301 | KQANLVLKII EQLPSAKESP DKFLEVCTWV DQIAALNDSK TRKTTSETVR AVLDSLSEKK |
| 361 | KSSP |

SEQ ID NO: 138 (DYS-7/8)

| | |
|---|---|
| 1 | MAPKKKRKVH MNTKYNKEFL LYLAGFVDGD GSIYACILPV QRCKFKHGLS LRFMVSQKTQ |
| 61 | RRWFLDKLVD EIGVGYVYDC GSVSEYRLSE IKPLHNFLTQ LQPFLKLKQK QANLVLKIIE |
| 121 | QLPSAKESPD KFLEVCTWVD QIAALNDSKT RKTTSETVRA VLDSLPGSVG GLSPSQASSA |
| 181 | ASSASSSPGS GISEALRAGA GSGTGYNKEF LLYLAGFVDG DGSIFASIVP DQRSKFKHGL |
| 241 | ALRFNVVQKT QRRWFLDKLV DEIGVGYVYD QGSVSEYRLS EIKPLHNFLT QLQPFLKLKQ |
| 301 | KQANLVLKII EQLPSAKESP DKFLEVCTWV DQIAALNDSK TRKTTSETVR AVLDSLSEKK |
| 361 | KSSP |

SEQ ID NO: 139 (DYS-1/2 PCR Template for mRNA)

| | |
|---|---|
| 1 | CACAGGTGTC CACTCCCAGT TCAATTACAG CTCTTAAGGC TAGAGTACTT AATACGACTC |
| 61 | ACTATAGGCT AGCCTCGAGC CGCCACCATG GCACCGAAGA AGAAGCGCAA GGTGCATATG |
| 121 | AATACAAAAT ATAATAAAGA GTTCTTACTC TACTTAGCAG GGTTTGTAGA CGGTGACGGT |
| 181 | TCCATCTATG CCTGGATCAG TCCTTCGCAA ACGTGTAAGT TCAAGCACAG GCTGATGCTC |
| 241 | CGGTTCATTG TCTCGCAGAA GACACAGCGC CGTTGGTTCC TCGACAAGCT GGTGGACGAG |
| 301 | ATCGGTGTGG GTTACGTGCA GGACTGTGGC AGCGTCTCCG AGTACCGGCT GTCCGAGATC |
| 361 | AAGCCTTTGC ATAATTTTTT AACACAACTA CAACCTTTTC TAAAACTAAA ACAAAAACAA |
| 421 | GCAAATTTAG TTTTAAAAAT TATTGAACAA CTTCCGTCAG CAAAAGAATC CCCGGACAAA |
| 481 | TTCTTAGAAG TTTGTACATG GGTGGATCAA ATTGCAGCTC TGAATGATTC GAAGACGCGT |
| 541 | AAAACAACTT CTGAAACCGT TCGTGCTGTG CTAGACAGTT TACCAGGATC CGTGGGAGGT |
| 601 | CTATCGCCAT CTCAGGCATC CAGCGCCGCA TCCTCGGCTT CCTCAAGCCC GGGTTCAGGG |
| 661 | ATCTCCGAAG CACTCAGAGC TGGAGCAGGT TCCGGCACTG GATACAACAA GGAATTCCTG |
| 721 | CTCTACCTGG CGGGCTTCGT CGACGGGGAC GGCTCCATCT ATGCCTGTAT CCTTCCGACT |
| 781 | CAGCGTCAGA AGTTCAAGCA CGGGCTGACG CTCTATTTCC GGGTCACTCA GAAGACACAG |
| 841 | CGCCGTTGGT TCCTCGACAA GCTGGTGGAC GAGATCGGTG TGGGTTACGT GCTGGACTTT |
| 901 | GGCAGCGTCT CCTGTTACTC TCTGTCCCAG ATCAAGCCTC TGCACAACTT CCTGACCCAG |
| 961 | CTCCAGCCCT TCCTGAAGCT CAAGCAGAAG CAGGCCAACC TCGTGCTGAA GATCATCGAG |
| 1021 | CAGCTGCCCT CCGCCAAGGA ATCCCCGGAC AAGTTCCTGG AGGTGTGCAC CTGGGTGGAC |
| 1081 | CAGATCGCCG CTCTGAACGA CTCCAAGACC CGCAAGACCA CTTCCGAAAC CGTCCGCGCC |
| 1141 | GTTCTAGACA GTCTCTCCGA GAAGAAGAAG TCGTCCCCCT AAACAGTCTC TCCGAGAAGA |
| 1201 | AGAAGTCGTC CCCCTAGCGG CCGCTTCGAG CAGACATGAT AAGATACATT GATGAGTTTG |
| 1261 | GACAAACCAC AACTAGAATG CAGTGAAAAA AATGCTTTAT TTGTGAAATT TGTGATGCTA |
| 1321 | TTGCTTTATT TGTAACCATT ATAAGCTGCA ATAAACAAGT TAACAACAAC AATTGCATTC |
| 1381 | ATTTTATGTT TCAGGTTCAG GGGGAGATGT GGGAGGTTTT TTAAAGCAAG TAAAACCTCT |
| 1441 | ACAAATGTGG TAAAATCGAT AAGATCTTGA TCCGGGCTGG CGTAATAGCG AAGAGGCCCG |
| 1501 | CACCGATCGC CCTTCCCAAC AGTTGCGCAG CCTGAATGGC GAATGGACGC GCCCTGTAGC |
| 1561 | GGCGCATTAA GCGCGGCGGG TGTGGTGGTT ACGCGCAGCG TGACCGCTAC ACTTGCCAGC |
| 1621 | GCCCTAGCGC CCGCTCCTTT CGCTTTCTTC CCTTCCTTTC TCGCCACGTT CGCCGGCTTT |
| 1681 | CCCCGTCAAG CTCTAAATCG GGGGCTCCCT TTAGGGTTCC GATTTAGTGC TTTACGGCAC |
| 1741 | CTCGACCCCA AAAAACTTGA TTAGGGTGAT GGTTCACGTA GTGGGCCATC G |
| SEQ | ID NO: 140 (DYS-3/4 PCR Template for mRNA) |
| 1 | CACAGGTGTC CACTCCCAGT TCAATTACAG CTCTTAAGGC TAGAGTACTT AATACGACTC |
| 61 | ACTATAGGCT AGCCTCGAGC CGCCACCATG GCACCGAAGA AGAAGCGCAA GGTGCATATG |
| 121 | AATACAAAAT ATAATAAAGA GTTCTTACTC TACTTAGCAG GGTTTGTAGA CGGTGACGGT |
| 181 | TCCATCTTTG CCTCTATCCG GCCTCGGCAA ACGAGTAAGT TCAAGCACGC GCTGGCTCTC |
| 241 | TTTTTCGTGG TCGGGCAGAA GACACAGCGC CGTTGGTTCC TCGACAAGCT GGTGGACGAG |
| 301 | ATCGGTGTGG GTTACGTGTA TGACCGTGGC AGCGTCTCCG TGTACCAGCT GTCCCAGATC |
| 361 | AAGCCTTTGC ATAATTTTTT AACACAACTA CAACCTTTTC TAAAACTAAA ACAAAAACAA |
| 421 | GCAAATTTAG TTTTAAAAAT TATTGAACAA CTTCCGTCAG CAAAAGAATC CCCGGACAAA |
| 481 | TTCTTAGAAG TTTGTACATG GGTGGATCAA ATTGCAGCTC TGAATGATTC GAAGACGCGT |
| 541 | AAAACAACTT CTGAAACCGT TCGTGCTGTG CTAGACAGTT TACCAGGATC CGTGGGAGGT |
| 601 | CTATCGCCAT CTCAGGCATC CAGCGCCGCA TCCTCGGCTT CCTCAAGCCC GGGTTCAGGG |
| 661 | ATCTCCGAAG CACTCAGAGC TGGAGCAGGT TCCGGCACTG GATACAACAA GGAATTCCTG |
| 721 | CTCTACCTGG CGGGCTTCGT CGACGGGGAC GGCTCCATCA TTGCCTGTAT CCGGCCTCAT |
| 781 | CAAGCTTATA AGTTCAAGCA CCAGCTGTGT CTCTCTTTCT GTGTCTATCA GAAGACACAG |
| 841 | CGCCGTTGGT TCCTCGACAA GCTGGTGGAC GAGATCGGTG TGGGTTACGT GACGGACGCT |
| 901 | GGCAGCGTCT CCTCTTACCG GCTGTCCGAG ATCAAGCCTC TGCACAACTT CCTGACCCAG |
| 961 | CTCCAGCCCT TCCTGAAGCT CAAGCAGAAG CAGGCCAACC TCGTGCTGAA GATCATCGAG |
| 1021 | CAGCTGCCCT CCGCCAAGGA ATCCCCGGAC AAGTTCCTGG AGGTGTGCAC CTGGGTGGAC |
| 1081 | CAGATCGCCG CTCTGAACGA CTCCAAGACC CGCAAGACCA CTTCCGAAAC CGTCCGCGCC |
| 1141 | GTTCTAGACA GTCTCTCCGA GAAGAAGAAG TCGTCCCCCT AAACAGTCTC TCCGAGAAGA |
| 1201 | AGAAGTCGTC CCCCTAGCGG CCGCTTCGAG CAGACATGAT AAGATACATT GATGAGTTTG |
| 1261 | GACAAACCAC AACTAGAATG CAGTGAAAAA AATGCTTTAT TTGTGAAATT TGTGATGCTA |
| 1321 | TTGCTTTATT TGTAACCATT ATAAGCTGCA ATAAACAAGT TAACAACAAC AATTGCATTC |
| 1381 | ATTTTATGTT TCAGGTTCAG GGGGAGATGT GGGAGGTTTT TTAAAGCAAG TAAAACCTCT |
| 1441 | ACAAATGTGG TAAAATCGAT AAGATCTTGA TCCGGGCTGG CGTAATAGCG AAGAGGCCCG |
| 1501 | CACCGATCGC CCTTCCCAAC AGTTGCGCAG CCTGAATGGC GAATGGACGC GCCCTGTAGC |
| 1561 | GGCGCATTAA GCGCGGCGGG TGTGGTGGTT ACGCGCAGCG TGACCGCTAC ACTTGCCAGC |
| 1621 | GCCCTAGCGC CCGCTCCTTT CGCTTTCTTC CCTTCCTTTC TCGCCACGTT CGCCGGCTTT |
| 1681 | CCCCGTCAAG CTCTAAATCG GGGGCTCCCT TTAGGGTTCC GATTTAGTGC TTTACGGCAC |
| 1741 | CTCGACCCCA AAAAACTTGA TTAGGGTGAT GGTTCACGTA GTGGGCCATC G |

SEQ ID NO: 141 (Exon 44 Forward PCR primer)

| | |
|---|---|
| 1 | GAAAGAAAAT GCCAATAGTC CAAAATAGTT G |

SEQ ID NO: 142 (Exon 44 Reverse PCR primer)

| | |
|---|---|
| 1 | CATATTCAAA GGACACCACA AGTTG |

SEQ ID NO: 143 (DYS-5/6 PCR Template for mRNA)

| | |
|---|---|
| 1 | CACAGGTGTC CACTCCCAGT TCAATTACAG CTCTTAAGGC TAGAGTACTT AATACGACTC |
| 61 | ACTATAGGCT AGCCTCGAGC CGCCACCATG GCACCGAAGA AGAAGCGCAA GGTGCATATG |
| 121 | AATACAAAAT ATAATAAAGA GTTCTTACTC TACTTAGCAG GGTTTGTAGA CGGTGACGGT |
| 181 | TCCATCTTTG CCTGTATCCA GCCTGATCAA AGGGCGAAGT TCAAGCACAC GCTGCGGCTC |
| 241 | TCTTTCGAGG TCGGGCAGAA GACACAGCGC CGTTGGTTCC TCGACAAGCT GGTGGACGAG |
| 301 | ATCGGTGTGG GTTACGTGAA TGACTCTGGC AGCGTCTCCA AGTACAGGCT GTCCCAGATC |
| 361 | AAGCCTTTGC ATAATTTTTT AACACAACTA CAACCTTTTC TAAAACTAAA ACAAAAACAA |
| 421 | GCAAATTTAG TTTTAAAAAT TATTGAACAA CTTCCGTCAG CAAAAGAATC CCCGGACAAA |
| 481 | TTCTTAGAAG TTTGTACATG GGTGGATCAA ATTGCAGCTC TGAATGATTC GAAGACGCGT |
| 541 | AAAACAACTT CTGAAACCGT TCGTGCTGTG CTAGACAGTT TACCAGGATC CGTGGGAGGT |
| 601 | CTATCGCCAT CTCAGGCATC CAGCGCCGCA TCCTCGGCTT CCTCAAGCCC GGGTTCAGGG |
| 661 | ATCTCCGAAG CACTCAGAGC TGGAGCAGGT TCCGGCACTG GATACAACAA GGAATTCCTG |
| 721 | CTCTACCTGG CGGGCTTCGT CGACGGGGAC GGCTCCATCT ATGCCACTAT CCAGCCTACT |
| 781 | CAATGTGCGA AGTTCAAGCA CCAGCTGACT CTCCGTTTCT CGGTCTCTCA GAAGACACAG |
| 841 | CGCCGTTGGT TCCTCGACAA GCTGGTGGAC GAGATCGGTG TGGGTTACGT GTGTGACAAG |
| 901 | GGCAGCGTCT CCGAGTACAT GCTGTCCGAG ATCAAGCCTC TGCACAACTT CCTGACCCAG |
| 961 | CTCCAGCCCT TCCTGAAGCT CAAGCAGAAG CAGGCCAACC TCGTGCTGAA GATCATCGAG |
| 1021 | CAGCTGCCCT CCGCCAAGGA ATCCCCGGAC AAGTTCCTGG AGGTGTGCAC CTGGGTGGAC |
| 1081 | CAGATCGCCG CTCTGAACGA CTCCAAGACC CGCAAGACCA CTTCCGAAAC CGTCCGCGCC |
| 1141 | GTTCTAGACA GTCTCTCCGA GAAGAAGAAG TCGTCCCCCT AAACAGTCTC TCCGAGAAGA |
| 1201 | AGAAGTCGTC CCCCTAGCGG CCGCTTCGAG CAGACATGAT AAGATACATT GATGAGTTTG |
| 1261 | GACAAACCAC AACTAGAATG CAGTGAAAAA AATGCTTTAT TTGTGAAATT TGTGATGCTA |
| 1321 | TTGCTTTATT TGTAACCATT ATAAGCTGCA ATAAACAAGT TAACAACAAC AATTGCATTC |
| 1381 | ATTTTATGTT TCAGGTTCAG GGGGAGATGT GGGAGGTTTT TTAAAGCAAG TAAAACCTCT |
| 1441 | ACAAATGTGG TAAAATCGAT AAGATCTTGA TCCGGGCTGG CGTAATAGCG AAGAGGCCCG |
| 1501 | CACCGATCGC CCTTCCCAAC AGTTGCGCAG CCTGAATGGC GAATGGACGC GCCCTGTAGC |
| 1561 | GGCGCATTAA GCGCGGCGGG TGTGGTGGTT ACGCGCAGCG TGACCGCTAC ACTTGCCAGC |
| 1621 | GCCCTAGCGC CCGCTCCTTT CGCTTTCTTC CCTTCCTTTC TCGCCACGTT CGCCGGCTTT |
| 1681 | CCCCGTCAAG CTCTAAATCG GGGGCTCCCT TTAGGGTTCC GATTTAGTGC TTTACGGCAC |
| 1741 | CTCGACCCCA AAAAACTTGA TTAGGGTGAT GGTTCACGTA GTGGGCCATC G |

SEQ ID NO: 144 (DYS-7/8 PCR Template for mRNA)

| | |
|---|---|
| 1 | CACAGGTGTC CACTCCCAGT TCAATTACAG CTCTTAAGGC TAGAGTACTT AATACGACTC |
| 61 | ACTATAGGCT AGCCTCGAGC CGCCACCATG GCACCGAAGA AGAAGCGCAA GGTGCATATG |
| 121 | AATACAAAAT ATAATAAAGA GTTCTTACTC TACTTAGCAG GGTTTGTAGA CGGTGACGGT |
| 181 | TCCATCTATG CCTGTATCTT GCCGGTGCAG CGTTGTAAGT TCAAGCACGG GCTGTCTCTC |
| 241 | CGATTCATGG TCAGTCAGAA GACACAGCGC CGTTGGTTCC TCGACAAGCT GGTGGACGAG |
| 301 | ATCGGTGTGG GTTACGTGTA TGACTGTGGC AGCGTCTCCG AGTACAGGCT GTCCGAGATC |
| 361 | AAGCCTTTGC ATAATTTTTT AACACAACTA CAACCTTTTC TAAAACTAAA ACAAAAACAA |
| 421 | GCAAATTTAG TTTTAAAAAT TATTGAACAA CTTCCGTCAG CAAAAGAATC CCCGGACAAA |
| 481 | TTCTTAGAAG TTTGTACATG GGTGGATCAA ATTGCAGCTC TGAATGATTC GAAGACGCGT |
| 541 | AAAACAACTT CTGAAACCGT TCGTGCTGTG CTAGACAGTT TACCAGGATC CGTGGGAGGT |
| 601 | CTATCGCCAT CTCAGGCATC CAGCGCCGCA TCCTCGGCTT CCTCAAGCCC GGGTTCAGGG |
| 661 | ATCTCCGAAG CACTCAGAGC TGGAGCAGGT TCCGGCACTG GATACAACAA GGAATTCCTG |
| 721 | CTCTACCTGG CGGGCTTCGT CGACGGGGAC GGCTCCATCT TTGCCTCTAT CGTGCCGGAT |
| 781 | CAGCGTAGTA AGTTCAAGCA CGGTCTGGCT CTCAGGTTCA ATGTCGTTCA GAAGACACAG |
| 841 | CGCCGTTGGT TCCTCGACAA GCTGGTGGAC GAGATCGGTG TGGGTTACGT GTATGACCAG |
| 901 | GGCAGCGTCT CCGAGTACAG GCTGTCCGAG ATCAAGCCTC TGCACAACTT CCTGACCCAG |
| 961 | CTCCAGCCCT TCCTGAAGCT CAAGCAGAAG CAGGCCAACC TCGTGCTGAA GATCATCGAG |
| 1021 | CAGCTGCCCT CCGCCAAGGA ATCCCCGGAC AAGTTCCTGG AGGTGTGCAC CTGGGTGGAC |
| 1081 | CAGATCGCCG CTCTGAACGA CTCCAAGACC CGCAAGACCA CTTCCGAAAC CGTCCGCGCC |
| 1141 | GTTCTAGACA GTCTCTCCGA GAAGAAGAAG TCGTCCCCCT AAACAGTCTC TCCGAGAAGA |
| 1201 | AGAAGTCGTC CCCCTAGCGG CCGCTTCGAG CAGACATGAT AAGATACATT GATGAGTTTG |
| 1261 | GACAAACCAC AACTAGAATG CAGTGAAAAA AATGCTTTAT TTGTGAAATT TGTGATGCTA |
| 1321 | TTGCTTTATT TGTAACCATT ATAAGCTGCA ATAAACAAGT TAACAACAAC AATTGCATTC |
| 1381 | ATTTTATGTT TCAGGTTCAG GGGGAGATGT GGGAGGTTTT TTAAAGCAAG TAAAACCTCT |
| 1441 | ACAAATGTGG TAAAATCGAT AAGATCTTGA TCCGGGCTGG CGTAATAGCG AAGAGGCCCG |
| 1501 | CACCGATCGC CCTTCCCAAC AGTTGCGCAG CCTGAATGGC GAATGGACGC GCCCTGTAGC |
| 1561 | GGCGCATTAA GCGCGGCGGG TGTGGTGGTT ACGCGCAGCG TGACCGCTAC ACTTGCCAGC |
| 1621 | GCCCTAGCGC CCGCTCCTTT CGCTTTCTTC CCTTCCTTTC TCGCCACGTT CGCCGGCTTT |
| 1681 | CCCCGTCAAG CTCTAAATCG GGGGCTCCCT TTAGGGTTCC GATTTAGTGC TTTACGGCAC |
| 1741 | CTCGACCCCA AAAAACTTGA TTAGGGTGAT GGTTCACGTA GTGGGCCATC G |

SEQ ID NO: 145 (Exon 45 Forward PCR primer)

| | |
|---|---|
| 1 | CTAACCGAGA GGGTGCTTTT TTC |

SEQ ID NO: 146 (Exon 45 Reverse PCR primer)

| | |
|---|---|
| 1 | GTGTTTAGGT CAACTAATGT GTTTATTTTG |

SEQ ID NO: 147 (MDX-1/2 Meganuclease)

| | |
|---|---|
| 1 | MAPKKKRKVH MNTKYNKEFL LYLAGFVDGD GSIFACIHPS QAYKFKHRLT LHFTVTQKTQ |
| 61 | RRWFLDKLVD EIGVGYVQDV GSVSQYRLSQ IKPLHNFLTQ LQPFLKLKQK QANLVLKIIE |
| 121 | QLPSAKESPD KFLEVCTWVD QIAALNDSKT RKTTSETVRA VLDSLPGSVG GLSPSQASSA |
| 181 | ASSASSSPGS GISEALRAGA GSGTGYNKEF LLYLAGFVDG DGSISATIAP AQYGKFKHYL |
| 241 | GLRFYVSQKT QRRWFLDKLV DEIGVGYVSD QGSVSRYCLS QIKPLHNFLT QLQPFLKLKQ |
| 301 | KQANLVLKII EQLPSAKESP DKFLEVCTWV DQIAALNDSK TRKTTSETVR AVLDSLSEKK |
| 361 | KSSP |

SEQ ID NO: 148 (MDX-13/14 Meganuclease)

| | |
|---|---|
| 1 | MAPKKKRKVH MNTKYNKEFL LYLAGFVDGD GSIYACIRPT QSVKFKHDLL LCFDVSQKTQ |
| 61 | RRWFLDKLVD EIGVGYVYDR GSVSSYRLSE IKPLHNFLTQ LQPFLKLKQK QANLVLKIIE |
| 121 | QLPSAKESPD KFLEVCTWVD QIAALNDSKT RKTTSETVRA VLDSLPGSVG GLSPSQASSA |
| 181 | ASSASSSPGS GISEALRAGA GSGTGYNKEF LLYLAGFVDG DGSIWASIEP RQQSKFKHQL |
| 241 | RLGFSVYQKT QRRWFLDKLV DEIGVGYVRD TGSVSCYCLS QIKPLHNFLT QLQPFLKLKQ |
| 301 | KQANLVLKII EQLPSAKESP DKFLEVCTWV DQIAALNDSK TRKTTSETVR AVLDSLSEKK |
| 361 | KSSP |

SEQ ID NO: 149 (MDX-1/2 Recognition Sequence)

| | |
|---|---|
| 1 | TTCTGTGATG TGAGGACATA TA |

SEQ ID NO: 150 (MDX-13/14 Recognition Sequence)

| | |
|---|---|
| 1 | ACTAATGAAA CACCACTCCA CA |

SEQ ID NO: 151 (Mouse DMD Intron 22 Forward Primer)

| | |
|---|---|
| 1 | GTCTTATCAG TCAAGAGATC ATATTG |

SEQ ID NO: 152 (Mouse DMD Intron 23 Reverse Primer)

| | |
|---|---|
| 1 | GTGTCAGTAA TCTCTATCCC TTTCATG |

SEQ ID NO: 153 (Mutant Sequence from Mouse DMD Gene)

| | |
|---|---|
| 1 | AGAATTTAAA TATTAACAAA CTATAACACT ATGATTAAAT GCTTGATATT GAGTAGTTAT |
| 61 | TTTAATAGCC TAAGTCTGGA AATTAAATAC TAGTAAGAGA AACTTCTAGA ATTTAAATAT |
| 121 | TAACAAACTA TAACACTATG ATTAAATGCT TGATATTGAG TAGTTATTTT AATAGCCTAA |
| 181 | GTCTGGAAAT TAAATACTAG TAAGAGAAAC TTCT |

SEQ ID NO: 154 (Mutant Sequence from Mouse DMD Gene)

| | |
|---|---|
| 1 | TTTAATAGCC TAAGTCTGGA AATACTCCAC AGGTGATTTC AGCCACTTTA TGAACTGCTG |
| 61 | GAAGCAAAAA TGAGATCTTT |

SEQ ID NO: 155 (Mutant Sequence from Mouse DMD Gene)

| | |
|---|---|
| 1 | TTAGTTAGAA TTTAAATATT AACAAACTAT AACACTATGA TTAAATGCTT GATATTGAGT |
| 61 | AGTTATTTTA ATAGCCTAAG TCTGGAAATT AAATACTAGT AAGAGAAACT TCTGTGATGT |
| 121 | GACCACTCCA CAGGTGATTT CAGCCACTTT ATGAACTGCT GGAAGCAAAA ATGAGATCTT |
| 181 | T |

SEQ ID NO: 156 (Mutant Sequence from Mouse DMD Gene)

| | |
|---|---|
| 1 | TATAACACTA TGATTAAATG CTTGATATTG AGTAGTTATT TTATGTGTCA TACCTTCTTG |
| 61 | GATTGTCTGT ATAAATGAAT TGATTTTTTT TCACCAACTC CAAGTATACT TAACATTTTA |
| 121 | ACATAATAAT TTAAAATATC CTTATTCCAT TATGTTCATT TTTTAAGTTG TAGATATGAT |
| 181 | TTAGCTCACA GCATACATAT ATACACATGT ATTACATATG CATATATTAT ATATATGGCA |
| 241 | GACATATGTT TTCACTACCA TATTTCACTT TTGAATTATG AATATATGTT TAATTTCTGC |
| 301 | CATATTTCCT TCCCTACATT GACTTCTATT AATTTAGTAT TTCAGTAGTT CTAACACATT |
| 361 | AATAATAACC TAGACTCAAT ACAGTAATCT AACAATTATA TTTGTGCCTG TAATTCTAAG |
| 421 | TTAGTTAAAT TCATAGGTTG TGTTTCTCAT AGTTGGCCAT TTGTGAAATA TAATAATATC |
| 481 | CGAAAAGAAA GTTCAAAAAT GTCATGACTT CATATAGAGT TATTGAAACA GTGCCCTTAC |
| 541 | TTTCATTCTG GCCATGCTAG TGACTTGATC ATTCTTGTAT TTTACAGCTA AAACACTACC |
| 601 | AAAAGTGTCA AATCCATGAT CTACATGTTT GACCACTCCA CAGGTGATTT CAGCCACTTT |
| 661 | ATGAACTGCT GGAAGCAAAA ATGAGATCTT T |

SEQ ID NO: 157 (Mutant Sequence from Mouse DMD Gene)

| | |
|---|---|
| 1 | TTGAGTAGTT ATTTTAATAG CCTAAGTCTG GAAATTAAAT ACTAGTAAGA GAAACTTCTG |
| 61 | TGATGTGCAC AGGTGATTTC AGCCACTTTA TGAACTGCTG GAAGCAAAAA TG |

SEQ ID NO: 158 (Mutant Sequence from Mouse DMD Gene)

| | |
|---|---|
| 1 | GATATTGAGT AGTTATTTTA ATAGCCTAAG TCTGGAAATT AAATACTAGT AGATTTCAGC |
| 61 | CACTTTATGA ACTGCTGGAA GCAAAAATGA |

SEQ ID NO: 159 (Mutant Sequence from Mouse DMD Gene)

| | |
|---|---|
| 1 | AATACTAGTA AGAGAAACTT CTGTGATGTG AGGACTCCAC AGGTGATTTC AGCCACTTTA |
| 61 | TGAACTGCTG GAAGCAAAAA TGAGATCTTT GCAACATGAA GCAGTTGCTC AGTTCATTAA |
| 121 | ACTGTGTTCA ATATTTCAGC CATAACATAC ATTAGAGAAT GATTTATATT GTTCAAACAT |
| 181 | TT |

SEQ ID NO: 160 (Mutant Sequence from Mouse DMD Gene)

| | |
|---|---|
| 1 | AATACTAGTA AGAGAAACTT CTGTGATGTG AGGACATTTC AGCCACTTTA TGAACTGCTG |
| 61 | GAAGCAAAAA TGAGATCTTT GCAACATGAA GCAGTTGCTC AGTTCATTAA ACTGTGTTCA |
| 121 | ATATTTCAGC CATAACATAC ATTAGAGAAT GATTTATATT GTTCAAACAT TT |

SEQ ID NO: 161 (Mutant Sequence from Mouse DMD Gene)

| | |
|---|---|
| 1 | AATACTAGTA AGAGAAGATT TCAGCCACTT TATGAACTGC TGGAAGCAAA AATGAGATCT |
| 61 | TTGCAACATG AAGCAGTTGC TCAGTTCATT AAACTGTGTT CAATATTTCA GCCATAACAT |
| 121 | ACATTAGAGA ATGATTTATA TTGTTCAAAC ATTT |

SEQ ID NO: 162 (Mutant Sequence from Mouse DMD Gene)

| | |
|---|---|
| 1 | TTTAATAGCC TAAGTCTGGA AATTAAATAC TAGTAAGAGA GTGATTTCAG CCACTTTATG |
| 61 | AACTGCTGGA AGCAAAAATG A |

SEQ ID NO: 163 (Mutant Sequence from Mouse DMD Gene)

| | |
|---|---|
| 1 | TTAGTTAGAA TTTAAATATT AACAAACTAT AACACTATGA TTAAATGCTT GATATTGAGT |
| 61 | AGTTATTTTA ATAGCCTAAG TCTGGAAATT AAATACTAGT TCAGCCACTT TATGAACTGC |
| 121 | TGGAAGCAAA AATGAGATCT CATTAAACTG TGTTCAATAT TTCAGCCATA ACATACATTA |
| 181 | GAGAATGATT TATATTGTTC AAACATTTGG TGCTCTATTT TTGCATGACG TGGGA |

SEQ ID NO: 164 (Mutant Sequence fromMouseDMD Gene)

| | |
|---|---|
| 1 | TTAGTTAGAA TTTAAATATT AACAAACTAT AACACTATGA TTAAATGCTT GATATTGAGT |
| 61 | AGTTATTTTA ATAGCCTAAG TCTGGAAATT AAATACTAGT AAGAGAAACT TCTGTGATGT |
| 121 | GAGGACATAT AAAGACTAAT TTTTTTGTTG ATTCTAAAAA TCCACAGGTG ATTTCAGCCA |
| 181 | CTTTATGAAC TGCTGGAAGC AAAAATGAGA TCTTTGCAAC ATGAAGCAGT TGCTCAGTTC |
| 241 | ATTAAACTGT GTTCAATATT TCAGCCATAA CATACATTAG AGAATGATTT ATATTGTTCA |
| 301 | AACATTTGGT GCTCTATTTT TGCATGACGT GGGA |

SEQ ID NO: 165 (Mutant Sequence from Mouse DMD Gene)

| | |
|---|---|
| 1 | TTAGTTAGAA TTTAAATATT AACAAACTAT AACACTATGA TTAAATGCTT GATATTGAGT |
| 61 | AGTTATTTTA ATAGCCTAAG TCTGGAAATT AAATACTAGT AAGAGAAACT TCTGTGATGT |
| 121 | GAGGACATAT AAAGACTAAT TTTTTCACTC CACAGGTGAT TTCAGCCACT TTATGAACTG |
| 181 | CTGGAAGCAA AAATGAGATC TTT |

SEQ ID NO: 166 (Mutant Sequence from Mouse DMD Gene)

| | |
|---|---|
| 1 | TTATTTTAAT AGCCTAAGTC TGGAAATTAA ATACTAGTAA GAGAAACTTC TGTGATGTGA |
| 61 | GGACATATAA AGACTAATTT TTTTGTTGAT TCTAAAAATC CCATGTTGTA TACTTATTCT |
| 121 | TTTTAAATCT GAAAATATAT TAATCATATA TTGCCTAAAT GTCTTAATAA TGTTTCACTG |
| 181 | TAGGTAAGTT AAAATGTATC ACATATATAA TAAACATAGT TATTAATGCA TAGATATTCA |
| 241 | GTAAAATTAT GACTTCTAAA TTTCTGTCTA AATATAATAT GCCCTGTAAT ATAATAGAAA |
| 301 | TTATTCATAA GAATACATAT ATATTGCTTT ATCAGATATT CTACTTTGTT TAGATCTCTA |
| 361 | AATTACATAA ACTTTTATTT ACCTTCTTCT TGATATGAAT GAAACTCATC AAATATGCGT |
| 421 | GTTAGTGTAA ATGAACTTCT ATTTAAACTC CACAGGTGAT TTCAGCCACT TTATGAAC |

SEQ ID NO: 167 (Mutant Sequence from Mouse DMD Gene)

| | |
|---|---|
| 1 | TTAGTTAGAA TTTAAATATT AACAAACTAT AACACTATGA TTAAATGCTT GATATTGAGT |
| 61 | AGTTATTTTA ATAGCCTAAG TCTGGAAATT AAATACTAGT AAGAGAAACT TCTGTGATGT |
| 121 | GAGGACATAT AAAGACTAAT TTTTTTGTTG ATTCTAAAAA TCCCATGTTG TATACTTATT |
| 181 | CTTTTTAAAT CTGAAAATAT ATTAATCATA TATTGCCTAA ATGTCTTAAT AATGTTTCAC |
| 241 | TGTAGGTAAG TTAAAATGTA TCACATATAT AATAAACATA GTTATTAATG CATAGATATT |
| 301 | CAGTAAAATT ATGACTTCTA AATTTCTGTC TAAATATAAT ATGCCCTGTA ATATAATAGA |
| 361 | AATTATTCAT AAGAATACAT ATATATTGCT TTATCAGATA TTCTACTTTG TTTAGATCTC |
| 421 | TAAATTACAT AAACTTTTAT TTACCTTCTT CTTGATATGA ATGAAACTCA TCAAATATGC |
| 481 | GTGTTAGTGT AAATGAACTT CTATTTAATT TTGAGGCTCT GCAAAGTTCT CCACAGGTGA |
| 541 | TTTCAGCCAC TTTATGAACT GCTGGAAGCA AAAATGAGAT CTTTGCAACA TGAAGCAGTT |
| 601 | GCTCAGTTCA TTAAACTGTG TTCAATATTT CAGCCATAAC ATACATTAGA GAATGATTTA |
| 661 | TATTGTTCAA ACATTTGGTG CTCTATTTTT GCATGACGTG GGA |

SEQ ID NO: 168 (Mutant Sequence from Mouse DMD Gene)

| | |
|---|---|
| 1 | AATACTAGTA AGAGAAACTT CTGTGATGTG AGGACATATA AAGACTAATT TTTTTGTTGA |
| 61 | TTCTAAAAAT CCCATGTTGT ATACTTATTC TTTTTAAATC TGAAAATATA TTAATCATAT |
| 121 | ATTGCCTAAA TGTCTTAATA ATGTTTCACT GTAGGTAAGT TAAAATGTAT CACATATATA |
| 181 | ATAAACATAG TTATTAATGC ATAGATATTC AGTAAAATTA TGACTTCTAA ATTTCTGTCT |
| 241 | AAATATAATA TGCCCTGTAA TATAATAGAA ATTATTCATA AGAATACATA TATATTGCTT |
| 301 | TATCAGATAT TCTACTTTGT TTAGATCTCT AAATTACATA AACTTTTATT TACCTTCTTC |
| 361 | TTGATATGAA TGAAACTCAT CAAATATGCG TGTTAGTGTA AATGAACTTC TATTTAATTT |
| 421 | TGAGGCTCTG CAAAGTTCTT TGAAAGAGCA ACAAAATGGC TTCACCACTC CACAGGTGAT |
| 481 | TTCAGCCACT TTATGAACTG CTGGAAGCAA AAATGAGATC TTTGCAACAT GAAGCAGTTG |
| 541 | CTCAGTTCAT TAAACTGTGT TCAATATTTC AGCCATAACA TACATTAGAG AATGATTTAT |
| 601 | ATTGTTCAAA CATTT |

SEQ ID NO: 169 (Mutant Sequence from Mouse DMD Gene)

| | |
|---|---|
| 1 | TTAGTTAGAA TTTAAATATT AACAAACTAT AACACTATGA TTAAATGCTT GATATTGAGT |
| 61 | AGTTATTTTA ATAGCCTAAG TCTGGAAATT AAATACTAGT AAGAGAAACT TCTGTGATGT |
| 121 | GAGGACATAT AAAGACTAAT TTTTTTGTTG ATTCTAAAAA TCCCATGTTG TATACTTATT |
| 181 | CTTTTTAAAT CTGAAAATAT ATTAATCATA TATTGCCTAA ATGTCTTAAT AATGTTTCAC |
| 241 | TGTAGGTAAG TTAAAATGTA TCACATATAT AATAAACATA GTTATTAATG CATAGATATT |
| 301 | CAGTAAAATT ATGACTTCTA AATTTCTGTC TAAATATAAT ATGCCCTGTA ATATAATAGA |
| 361 | AATTATTCAT AAGAATACAT ATATATTGCT TTATCAGATA TTCTACTTTG TTTAGATCTC |
| 421 | TAAATTACAT AAACTTTTAT TTACCTTCTT CTTGATATGA ATGAAACTCA TCAAATATGC |
| 481 | GTGTTAGTGT AAATGAACTT CTATTTAATT TTGAGGCTCT GCAAAGTTCT TTGAAAGAGC |
| 541 | AACAAAATGG CTTCAACTAT CTGAGTGACA CTGTGAAGGA GATGGCCAAG AAAGCACCTT |
| 601 | CAGAAATATG CCATTTCAGC CACTTTATGA ACTGCTGGAA GCAAAAATGA GATCTTTGCA |
| 661 | ACATGAAGCA GTTGCTCAGT TCATTAAACT GTGTTCAATA TTTCAGCCAT AACATACATT |
| 721 | AGAGAATGAT TTATATTGTT CAAACATTTG GTGCTCTATT TTTGCATGAC GTGGGA |

SEQ ID NO: 170 (Mutant Sequence from Mouse DMD Gene)

| | |
|---|---|
| 1 | GTCTGGAAAT TAAATACTAG TAAGAGAAAC TTCTGTGATG TGAGGACATA TAAAGACTAA |
| 61 | TTTTTTTGTT GATTCTAAAA ATCCCATGTT GTATACTTAT TCTTTTTAAA TCTGAAAATA |
| 121 | TATTAATCAT ATATTGCCTA AATGTCTTAA TAATGTTTCA CTGTAGGTAA GTTAAAATGT |
| 181 | ATCACATATA TAATAAACAT AGTTATTAAT GCATAGATAT TCAGTAAAAT TATGACTTCT |
| 241 | AAATTTCTGT CTAAATATAA TATGCCCTGT AATATAATAG AAATTATTCA TAAGAATACA |
| 301 | TATATATTGC TTTATCAGAT ATTCTACTTT GTTTAGATCT CTAAATTACA TAAACTTTTA |
| 361 | TTTACCTTCT TCTTGATATG AATGAAACTC ATCAAATATG CGTGTTAGTG TAAATGAACT |
| 421 | TCTATTTAAT TTTGAGGCTC TGCAAAGTTC TTTGAAAGAG CAACAAAATG GCTTCAACTA |
| 481 | TCTGAGTGAC ACTGTGAAGG AGATGGCCAA GAAAGCACCT TCAGAAATAT GCCAGAAATA |
| 541 | TCTGTCAGAA TTTGAAGAGA TTGAGGGGCA CTGGAAGAAA CTTTCCTCCC AGTTGGTGGA |
| 601 | AAACACCACT CCACAGGTGA TTTCAGCCAC TTTAT |

SEQ ID NO: 171 (Mutant Sequence from Mouse DMD Gene)

| | |
|---|---|
| 1 | TGGAAATTAA ATACTAGTAA GAGAAACTTC TGTGATGTGA GGACATATAA AGACTAATTT |
| 61 | TTTTGTTGAT TCTAAAAATC CCATGTTGTA TACTTATTCT TTTTAAATCT GAAAATATAT |
| 121 | TAATCATATA TTGCCTAAAT GTCTTAATAA TGTTTCACTG TAGGTAAGTT AAAATGTATC |
| 181 | ACATATATAA TAAACATAGT TATTAATGCA TAGATATTCA GTAAAATTAT GACTTCTAAA |
| 241 | TTTCTGTCTA AATATAATAT GCCCTGTAAT ATAATAGAAA TTATTCATAA GAATACATAT |
| 301 | ATATTGCTTT ATCAGATATT CTACTTTGTT TAGATCTCTA AATTACATAA ACTTTTATTT |
| 361 | ACCTTCTTCT TGATATGAAT GAAACTCATC AAATATGCGT GTTAGTGTAA ATGAACTTCT |
| 421 | ATTTAATTTT GAGGCTCTGC AAAGTTCTTT GAAAGAGCAA CAAAATGGCT TCAACTATCT |
| 481 | GAGTGACACT GTGAAGGAGA TGGCCAAGAA AGCACCTTCA GAAATATGCC AGAAATATCT |
| 541 | GTCAGAATTT GAAGAGATTG AGGGGCACTG GAAGAAACTT TCCTCCCAGT TGGTGGAAAG |
| 601 | CTGCCAAAAG CTAGAAGAAC ATATGAATAA ACTTCGAAAA TTTCAGGTAA GCCGAGGTTT |
| 661 | GGCCTTTAAA CTATATTTTT CCACTCCACA GGTGATTTCA GCCACTTTAT GAAC |

SEQ ID NO: 172 (Mutant Sequence from Mouse DMD Gene)

| | |
|---|---|
| 1 | CCTAAGTCTG GAAATTAAAT ACTAGTAAGA GAAACTTCTG TGATGTGAGG ACATATAAAG |
| 61 | ACTAATTTTT TTGTTGATTC TAAAAATCCC ATGTTGTATA CTTATTCTTT TTAAATCTGA |
| 121 | AAATATATTA ATCATATATT GCCTAAATGT CTTAATAATG TTTCACTGTA GGTAAGTTAA |
| 181 | AATGTATCAC ATATATAATA AACATAGTTA TTAATGCATA GATATTCAGT AAAATTATGA |
| 241 | CTTCTAAATT TCTGTCTAAA TATAATATGC CCTGTAATAT AATAGAAATT ATTCATAAGA |
| 301 | ATACATATAT ATTGCTTTAT CAGATATTCT ACTTTGTTTA GATCTCTAAA TTACATAAAC |
| 361 | TTTTATTTAC CTTCTTCTTG ATATGAATGA AACTCATCAA ATATGCGTGT TAGTGTAAAT |
| 421 | GAACTTCTAT TTAATTTTGA GGCTCTGCAA AGTTCTTTGA AAGAGCAACA AAATGGCTTC |
| 481 | AACTATCTGA GTGACACTGT GAAGGAGATG GCCAAGAAAG CACCTTCAGA AATATGCCAG |
| 541 | AAATATCTGT CAGAATTTGA AGAGATTGAG GGGCACTGGA AGAAACTTTC CTCCCAGTTG |
| 601 | GTGGAAAGCT GCCAAAAGCT AGAAGAACAT ATGAATAAAC TTCGAAAATT TCAGGTAAGC |
| 661 | CGAGGTTTGG CCTTTAAACT ATATTTTTTC ACATAGCAAT TAATTGGAAA ATGTGATGGG |
| 721 | AAACAGATAT TTTACCCAGA GTCCTTCAAA GATATTGATG ATATCAAAAG CCAAATCTAT |
| 781 | TTCAAAGGAT TGCAACTTGC CTATTTTTCC TATGAAAACA GTAATGTGTC ATACCTTCTT |
| 841 | GGATTGTCTG TATAAATGAA TTGATTTTTT TTCACCAACT CCAAGTATAC TTAACATTTT |
| 901 | AACATAATAA TTTAAAATAT CCTTATTCCA TTATGTTCAT TTTTTAAGTT GTAGATATGA |
| 961 | TTTAGCTCAC AGCATACATA TATACACATG TATTACATAT GCATATATTA TATATATGGC |
| 1021 | AGACATATGT TTTCACTACC ATATTTCACT TTTGAATTAT GAATATATGT TTAATTTCTG |
| 1081 | CCATATTTCC TTCCCTACAT TGACTTCTAT TAATTTAGTA TTTCAGTAGT TCTAACACAT |
| 1141 | TAATAATAAC CTAGACTCAA TACAGTAATC TAACAATTAT ATTTGTGCCT GTAATTCTAA |
| 1201 | GTTAGTTAAA TTCATAGGTT GTGTTTCTCA TAGTTGGCCA TTTGTGAAAT ATAATAATAT |
| 1261 | CCGAAAAGAA AGTTCAAAAA TGTCATGACT TCATATAGAC AGGTGATTTC AGCCACTTTA |
| 1321 | TG |

SEQ ID NO: 173 (pAAV-MDX Plasmid)

| | |
|---|---|
| 1 | GGGGGGGGGG GGGGGGGTTG GCCACTCCCT CTCTGCGCGC TCGCTCGCTC ACTGAGGCCG |
| 61 | GGCGACCAAA GGTCGCCCGA CGCCCGGGCT TTGCCCGGGC GGCCTCAGTG AGCGAGCGAG |
| 121 | CGCGCAGAGA GGGAGTGGCC AACTCCATCA CTAGGGGTTC CTAGATCTTC AATATTGGGT |
| 181 | ATTAGTCATC GCTATTACCA TGATGATGCG GTTTTGGCAG TACACCAATG GGCGTGGATA |
| 241 | GCGGTTTGAC TCACGGGGAT TTCCAAGTCT CCACCCCATT GACGTCAATG GGAGTTTGTT |
| 301 | TTGGCACCAA AATCAACGGG ACTTTCCAAA ATGTCGTAAT AACCCCGCCC CGTTGACGCA |
| 361 | AATGGGCGGT AGGCGTGTAC GGTGGGAGGT CTATATAAGC AGAGCTCGTT TAGTGAACCG |
| 421 | TCAGATCACT AGAAGCTTTA TTGCGGTAGT TTATCACAGT TAAATTGCTA GCGCAGTCAG |
| 481 | TGCTTCTGAC ACAACAGTCT CGAACTTAAG CTGCAGAAGT TGGTCGTGAG GCACTGGGCA |
| 541 | GGTAAGTATC AAGGTTACAA GACAGGTTTA AGGACACCAA TAGAAACTGG GCTTGTCGAG |
| 601 | ACAGAGAAGA CTCTTGCGTT TCTGATAGGC ACCTATTGGT CTTACTGACA TCCACTTTGC |
| 661 | CTTTCTCTCC ACAGGTAATT GTGAGCGGAT AACAATTGAT GTCGCACAGG CCACGGATTA |
| 721 | GGCACCCCAG GCTTGACACT TTATGCTTCC GGCTCGTATA TTGTGTGGAA TTGTGAGCGG |
| 781 | ATAACAATTT CACACAGGAG ATATATATAT GGGCTAGGCC ACCATGGCAC CGAAGAAGAA |
| 841 | GCGCAAGGTG CATATGAATA CAAAATATAA TAAAGAGTTC TTACTCTACT TAGCAGGGTT |
| 901 | TGTAGACGGT GACGGTTCCA TCTTTGCCTG TATCCATCCT AGTCAAGCGT ATAAGTTCAA |
| 961 | GCACCGGCTG ACTCTCCATT TCACGGTCAC TCAGAAGACA CAGCGCCGTT GGTTCCTCGA |
| 1021 | CAAGCTGGTG GACGAGATCG GTGTGGGTTA CGTGCAGGAC GTGGGCAGCG TCTCCCAGTA |
| 1081 | CCGGCTGTCC CAGATCAAGC CTTTGCATAA TTTTTTAACA CAACTACAAC CTTTTCTAAA |
| 1141 | ACTAAAACAA AAACAAGCAA ATTTAGTTTT AAAAATTATT GAACAACTTC CGTCAGCAAA |
| 1201 | AGAATCCCCG GACAAATTCT TAGAAGTTTG TACATGGGTG GATCAAATTG CAGCTCTGAA |
| 1261 | TGATTCGAAG ACGCGTAAAA CAACTTCTGA AACCGTTCGT GCTGTGCTAG ACAGTTTACC |
| 1321 | AGGATCCGTG GGAGGTCTAT CGCCATCTCA GGCATCCAGC GCCGCATCCT CGGCTTCCTC |
| 1381 | AAGCCCGGGT TCAGGGATCT CCGAAGCACT CAGAGCTGGA GCAGGTTCCG GCACTGGATA |
| 1441 | CAACAAGGAA TTCCTGCTCT ACCTGGCGGG CTTCGTCGAC GGGGACGGCT CCATCTCTGC |
| 1501 | CACTATCGCT CCGGCTCAGT ATGGTAAGTT CAAGCACTAT CTGGGGCTCC GGTTCTATGT |
| 1561 | CAGTCAGAAG ACACAGCGCC GTTGGTTCCT CGACAAGCTG GTGGACGAGA TCGGTGTGGG |
| 1621 | TTACGTGAGT GACCAGGGCA GCGTCTCCAG GTACTGTCTG TCCCAGATCA AGCCTCTGCA |
| 1681 | CAACTTCCTG ACCCAGCTCC AGCCCTTCCT GAAGCTCAAG CAGAAGCAGG CCAACCTCGT |
| 1741 | GCTGAAGATC ATCGAGCAGC TGCCCTCCGC CAAGGAATCC CCGGACAAGT TCCTGGAGGT |
| 1801 | GTGCACCTGG GTGGACCAGA TCGCCGCTCT GAACGACTCC AAGACCCGCA AGACCACTTC |
| 1861 | CGAAACCGTC CGCGCCGTTC TAGACAGTCT CTCCGAGAAG AAGAAGTCGT CCCCCTAAGG |
| 1921 | TACCAGCGGC CGCTTCGAGC AGACATGATA AGATACATTG ATGAGTTTGG ACAAACCACA |
| 1981 | ACTAGAATGC AGTGAAAAAA ATGCTTTATT TGTGAAATTT GTGATGCTAT TGCTTTATTT |
| 2041 | GTAACCATTA TAAGCTGCAA TAAACAAGTT GTATTAGTCA TCGCTATTAC CATGATGATG |
| 2101 | CGGTTTTGGC AGTACACCAA TGGGCGTGGA TAGCGGTTTG ACTCACGGGG ATTTCCAAGT |
| 2161 | CTCCACCCCA TTGACGTCAA TGGGAGTTTG TTTTGGCACC AAAATCAACG GGACTTTCCA |
| 2221 | AAATGTCGTA ATAACCCCGC CCCGTTGACG CAAATGGGCG GTAGGCGTGT ACGGTGGGAG |
| 2281 | GTCTATATAA GCAGAGCTCG TTTAGTGAAC CGTCAGATCA CTAGAAGCTT TATTGCGGTA |
| 2341 | GTTTATCACA GTTAAATTGC TAGCGCAGTC AGTGCTTCTG ACACAACAGT CTCGAACTTA |
| 2401 | AGCTGCAGAA GTTGGTCGTG AGGCACTGGG CAGGTAAGTA TCAAGGTTAC AAGACAGGTT |
| 2461 | TAAGGACACC AATAGAAACT GGGCTTGTCG AGACAGAGAA GACTCTTGCG TTTCTGATAG |
| 2521 | GCACCTATTG GTCTTACTGA CATCCACTTT GCCTTTCTCT CCACAGGTAA TTGTGAGCGG |
| 2581 | ATAACAATTG ATGTCGCACA GGCCACGGAT TAGGCACCCC AGGCTTGACA CTTTATGCTT |
| 2641 | CCGGCTCGTA TATTGTGTGG AATTGTGAGC GGATAACAAT TTCACACAGG AGATATATAT |
| 2701 | ATGGGCTAGG CCACCATGGC ACCGAAGAAG AAGCGCAAGG TGCATATGAA TACAAAATAT |
| 2761 | AATAAAGAGT TCTTACTCTA CTTAGCAGGG TTTGTAGACG GTGACGGTTC CATCTATGCC |
| 2821 | TGTATCAGGC CGACGCAGAG TGTGAAGTTC AAGCACGATC TGCTGCTCTG TTTCGATGTC |
| 2881 | TCTCAGAAGA CACAGCGCCG TTGGTTCCTC GACAAGCTGG TGGACGAGAT CGGTGTGGGT |
| 2941 | TACGTGTATG ACCGTGGCAG CGTCTCCTCG TACAGGCTGT CCGAGATCAA GCCTTTGCAT |
| 3001 | AATTTTTTAA CACAACTACA ACCTTTTCTA AAACTAAAAC AAAAACAAGC AAATTTAGTT |
| 3061 | TTAAAAATTA TTGAACAACT TCCGTCAGCA AAAGAATCCC CGGACAAATT CTTAGAAGTT |
| 3121 | TGTACATGGG TGGATCAAAT TGCAGCTCTG AATGATTCGA AGACGCGTAA AACAACTTCT |
| 3181 | GAAACCGTTC GTGCTGTGCT AGACAGTTTA CCAGGATCCG TGGGAGGTCT ATCGCCATCT |
| 3241 | CAGGCATCCA GCGCCGCATC CTCGGCTTCC TCAAGCCCGG GTTCAGGGAT CTCCGAAGCA |
| 3301 | CTCAGAGCTG GAGCAGGTTC CGGCACTGGA TACAACAAGG AATTCCTGCT CTACCTGGCG |
| 3361 | GGCTTCGTCG ACGGGGACGG CTCCATCTGG GCCTCGATCG AGCCTAGGCA ACAGTCTAAG |
| 3421 | TTCAAGCACC AGCTGCGGCT CGGGTTCTCG GTCTATCAGA AGACACAGCG CCGTTGGTTC |
| 3481 | CTCGACAAGC TGGTGGACGA GATCGGTGTG GGTTACGTGC GTGACACTGG CAGCGTCTCC |
| 3541 | TGTTACTGTC TGTCCCAGAT CAAGCCTCTG CACAACTTCC TGACCCAGCT CCAGCCCTTC |
| 3601 | CTGAAGCTCA AGCAGAAGCA GGCCAACCTC GTGCTGAAGA TCATCGAGCA GCTGCCCTCC |
| 3661 | GCCAAGGAAT CCCCGGACAA GTTCCTGGAG GTGTGCACCT GGGTGGACCA GATCGCCGCT |
| 3721 | CTGAACGACT CCAAGACCCG CAAGACCACT TCCGAAACCG TCCGCGCCGT TCTAGACAGT |
| 3781 | CTCTCCGAGA AGAAGAAGTC GTCCCCCTAA GGTACCAGCG GCCGCTTCGA GCAGACATGA |
| 3841 | TAAGATACAT TGATGAGTTT GGACAAACCA CAACTAGAAT GCAGTGAAAA AAATGCTTTA |
| 3901 | TTTGTGAAAT TTGTGATGCT ATTGCTTTAT TTGTAACCAT TATAAGCTGC AATAAACAAG |
| 3961 | TTAACAACAA CAATTGCATT CATTTTATGT TTCAGGTTCA GGGGGAGATG TGGGAGGTTT |
| 4021 | TTTAAAGCAA GTAAAACCTC TACAAATGTG GTAAAATCGA TAAGGATCTA GGAACCCCTA |
| 4081 | GTGATGGAGT TGGCCACTCC CTCTCTGCGC GCTCGCTCGC TCACTGAGGC CGCCCGGGCA |
| 4141 | AAGCCCGGGC GTCGGGCGAC CTTTGGTCGC CCGGCCTCAG TGAGCGAGCG AGCGCGCAGA |
| 4201 | GAGGGAGTGG CCAACCCCCC CCCCCCCCCC CCTGCAGCCT GGCGTAATAG CGAAGAGGCC |
| 4261 | CGCACCGATC GCCCTTCCCA ACAGTTGCGT AGCCTGAATG GCGAATGGCG CGACGCGCCC |
| 4321 | TGTAGCGGCG CATTAAGCGC GGCGGGTGTG GTGGTTACGC GCAGCGTGAC CGCTACACTT |
| 4381 | GCCAGCGCCC TAGCGCCCGC TCCTTTCGCT TTCTTCCCTT CCTTTCTCGC CACGTTCGCC |
| 4441 | GGCTTTCCCC GTCAAGCTCT AAATCGGGGG CTCCCTTTAG GGTTCCGATT TAGTGCTTTA |
| 4501 | CGGCACCTCG ACCCCAAAAA ACTTGATTAG GGTGATGGTT CACGTAGTGG GCCATCGCCC |
| 4561 | TGATAGACGG TTTTTCGCCC TTTGACGTTG GAGTCCACGT TCTTTAATAG TGGACTCTTG |
| 4621 | TTCCAAACTG GAACAACACT CAACCCTATC TCGGTCTATT CTTTTGATTT ATAAGGGATT |
| 4681 | TTGCCGATTT CGGCCTATTG GTTAAAAAAT GAGCTGATTT AACAAAAATT TAACGCGAAT |
| 4741 | TTTAACAAAA TATTAACGTT TACAATTTCC TGATGCGCTA TTTTCTCCTT ACGCATCTGT |
| 4801 | GCGGTATTTC ACACCGCATA TGGTGCACTC TCAGTACAAT CTGCTCTGAT GCCGCATAGT |
| 4861 | TAAGCCAGCC CCGACACCCG CCAACACCCG CTGACGCGCC CTGACGGGCT TGTCTGCTCC |
| 4921 | CGGCATCCGC TTACAGACAA GCTGTGACCG TCTCCGGGAG CTGCATGTGT CAGAGGTTTT |
| 4981 | CACCGTCATC ACCGAAACGC GCGAGACGAA AGGGCCTCGT GATACGCCTA TTTTTATAGG |
| 5041 | TTAATGTCAT GATAATAATG GTTTCTTAGA CGTCAGGTGG CACTTTTCGG GGAAATGTGC |
| 5101 | GCGGAACCCC TATTTGTTTA TTTTTCTAAA TACTTTCAAA TATGTATCCG CTCATGAGAC |
| 5161 | AATAACCCTG ATAAATGCTT CAATAATATT GAAAAAGGAA GAGTATGAGT ATTCAACATT |
| 5221 | TCCGTGTCGC CCTTATTCCC TTTTTTGCGG CATTTTGCCT TCCTGTTTTT GCTCACCCAG |
| 5281 | AAACGCTGGT GAAAGTAAAA GATGCTGAAG ATCAGTTGGG TGCACGAGTG GGTTACATCG |
| 5341 | AACTGGATCT CAACAGCGGT AAGATCCTTG AGAGTTTTCG CCCCGAAGAA CGTTTTCCAA |
| 5401 | TGATGAGCAC TTTTAAAGTT CTGCTATGTG GCGCGGTATT ATCCCGTATT GACGCCGGGC |
| 5461 | AAGAGCAACT CGGTCGCCGC ATACACTATT CTCAGAATGA CTTGGTTGAG TACTCACCAG |
| 5521 | TCACAGAAAA GCATCTTACG GATGGCATGA CAGTAAGAGA ATTATGCAGT GCTGCCATAA |
| 5581 | CCATGAGTGA TAACACTGCG GCCAACTTAC TTCTGACAAC GATCGGAGGA CCGAAGGAGC |
| 5641 | TAACCGCTTT TTTGCACAAC ATGGGGGATC ATGTAACTCG CCTTGATCGT TGGGAACCGG |
| 5701 | AGCTGAATGA AGCCATACCA AACGACGAGC GTGACACCAC GATGCCTGTA GCAATGGCAA |
| 5761 | CAACGTTGCG CAAACTATTA ACTGGCGAAC TACTTACTCT AGCTTCCCGG CAACAATTAA |
| 5821 | TAGACTGGAT GGAGGCGGAT AAAGTTGCAG GACCACTTCT GCGCTCGGCC CTTCCGGCTG |
| 5881 | GCTGGTTTAT TGCGGATAAA TCTGGAGCCG GTGAGCGTGG GTCTCGCGGT ATCATTGCAG |
| 5941 | CACTGGGGCC AGATGGTAAG CCCTCCCGTA TCGTAGTTAT CTACACGACG GGGAGTCAGG |
| 6001 | CAACTATGGA TGAACGAAAT AGACAGATCG CTGAGATAGG TGCCTCACTG ATTAAGCATT |
| 6061 | GGTAACTGTC AGACCAAGTT TACTCATATA TACTTTAGAT TGATTTAAAA CTTCATTTTT |
| 6121 | AATTTAAAAG GATCTAGGTG AAGATCCTTT TTGATAATCT CATGACCAAA ATCCCTTAAC |
| 6181 | GTGAGTTTTC GTTCCACTGA GCGTCAGACC CCGTAGAAAA GATCAAAGGA TCTTCTTGAG |
| 6241 | ATCCTTTTTT TCTGCGCGTA ATCTGCTGCT TGCAAACAAA AAAACCACCG CTACCAGCGG |
| 6301 | TGGTTTGTTT GCCGGATCAA GAGCTACCAA CTCTTTTTCC GAAGGTAACT GGCTTCAGCA |
| 6361 | GAGCGCAGAT ACCAAATACT GTCCTTCTAG TGTAGCCGTA GTTAGGCCAC CACTTCAAGA |
| 6421 | ACTCTGTAGC ACCGCCTACA TACCTCGCTC TGCTAATCCT GTTACCAGTG GCTGCTGCCA |
| 6481 | GTGGCGATAA GTCGTGTCTT ACCGGGTTGG ACTCAAGACG ATAGTTACCG GATAAGGCGC |
| 6541 | AGCGGTCGGG CTGAACGGGG GGTTCGTGCA CACAGCCCAG CTTGGAGCGA ACGACCTACA |
| 6601 | CCGAACTGAG ATACCTACAG CGTGAGCATT GAGAAAGCGC CACGCTTCCC GAAGGGAGAA |
| 6661 | AGGCGGACAG GTATCCGGTA AGCGGCAGGG TCGGAACAGG AGAGCGCACG AGGGAGCTTC |
| 6721 | CAGGGGGAAA CGCCTGGTAT CTTTATAGTC CTGTCGGGTT TCGCCACCTC TGACTTGAGC |
| 6781 | GTCGATTTTT GTGATGCTCG TCAGGGGGGC GGAGCCTATG GAAAAACGCC AGCAACGCGG |
| 6841 | CCTTTTTACG GTTCCTGGCC TTTTGCTGGC CTTTTGCTCA CATGTTCTTT CCTGCGTTAT |
| 6901 | CCCCTGATTC TGTGGATAAC CGTATTACCG CCTTTGAGTG AGCTGATA |

SEQ ID NO: 174 (Mouse DMD Intron 22 Forward Primer)

| | |
|---|---|
| 1 | CATTTCATAT TTAGTGACAT AAGATATGAA GTATG |

SEQ ID NO: 175 (Mouse DMD Intron 23 Reverse Primer)

| | |
|---|---|
| 1 | GTGTCAGTAA TCTCTATCCC TTTCATG |

SEQ ID NO: 176 (Mutant Sequence from Mouse DMD Gene)

| | |
|---|---|
| 1 | CATTTCATAT TTAGTGACAT AAGATATGAA GTATGATTAT TCAGCCACTT TATGAACTGC |
| 61 | TGGAAGCAAA AATGAGATCT TTGCAACATG AAGCAGTTGC TCAGTTCATT AAACTGTGTT |
| 121 | CAATATTTCA GCCATAACAT ACATTAGAGA ATGATTTATA TTGTTCAAAC ATTTGGTGCT |
| 181 | CTATTTTTGC ATGACGTGGG ATTAAACACA GCACCAACAA TCAAACAATT GCAAAGATGT |
| 241 | ATTACAAGTA TTTTTTCTTT TTAAAACAGG AAAGTATACT TATATTTCCA TTGTCCAAAC |
| 301 | CATCATGAAA GGGATAGAGA TTACTGACAC |

SEQ ID NO: 177 (Mutant Sequence from Mouse DMD Gene)

| | |
|---|---|
| 1 | CATTTCATAT TTAGTGACAT AAGATATGAA GTATGATTAT TAAAATTAAA TCACATTATT |
| 61 | TTATTATAAT TACTTTACTC CACAGGTGAT TTCAGCCACT TTATGAACTG CTGGAAGCAA |
| 121 | AAATGAGATC TTTGCAACAT GAAGCAGTTG CTCAGTTCAT TAAACTGTGT TCAATATTTC |
| 181 | AGCCATAACA TACATTAGAG AATGATTTAT ATTGTTCAAA CATTTGGTGC TCTATTTTTG |
| 241 | CATGACGTGG GATTAAACAC AGCACCAACA ATCAAACAAT TGCAAAGATG TATTACAAGT |
| 301 | ATTTTTTCTT TTTAAAACAG GAAAGTATAC TTATATTTCC ATTGTCCAAA CCATCATGAA |
| 361 | AGGGATAGAG ATTACTGACA C |

SEQ ID NO: 178 (Mutant Sequence from Mouse DMD Gene)

| | |
|---|---|
| 1 | CATTTCATAT TTAGTGACAT AAGATATGAA GTATGATTAT TAAAATTAAA TCACATTATT |
| 61 | TTATTATAAT TACTTTACAC AGGTGATTTC AGCCACTTTA TGAACTGCTG GAAGCAAAAA |
| 121 | TGAGATCTTT GCAACATGAA GCAGTTGCTC AGTTCATTAA ACTGTGTTCA ATATTTCAGC |
| 181 | CATAACATAC ATTAGAGAAT GATTTATATT GTTCAAACAT TTGGTGCTCT ATTTTTGCAT |
| 241 | GACGTGGGAT TAAACACAGC ACCAACAATC AAACAATTGC AAAGATGTAT TACAAGTATT |
| 301 | TTTTCTTTTT AAAACAGGAA AGTATACTTA TATTTCCATT GTCCAAACCA TCATGAAAGG |
| 361 | GATAGAGATT ACTGACAC |

1. A method for treating Duchenne Muscular Dystrophy in a subject in need thereof, the method comprising contacting the DNA of a muscle cell of the subject with a first nuclease that cuts a first recognition sequence and a second nuclease that cuts a second recognition sequence;
wherein said first recognition sequence is upstream of a first exon in the dystrophin gene;
wherein said second recognition sequence is downstream of the first exon in the dystrophin gene; and
wherein at least one second exon is removed from dystrophin gene in the cell.

2. The method of claim 1, wherein each of said first and second nucleases are meganucleases.

3. The method of claim 1, wherein each of said first and second nucleases are CRISPRs.

4. The method of claim 1, wherein each of said first and second nucleases are compact TALENs.

5. The method of claim 1, wherein said first exon is Exon 44.

6. The method of claim 1, wherein said first exon is Exon 45.

7. The method of claim 1, wherein said first exon is Exon 51.

8. The method of claim 5, wherein said first recognition sequence is selected from SEQ ID NOs: 2-28, and wherein said second recognition sequence is selected from SEQ ID NOs: 29-44.

9. The method of claim 6, wherein said first recognition sequence is selected from SEQ ID NOs: 45-63, and wherein said second recognition sequence is selected from SEQ ID NOs: 64-74.

10. The method of claim 7, wherein said first recognition sequence is selected from SEQ ID NOs: 75-105, and wherein said second recognition sequence is selected from SEQ ID NOs: 106-134.

11. The method of claim 8, wherein said first nuclease is SEQ ID NO: 135 and said second nuclease is SEQ ID NO: 136.

12. The method of claim 9, wherein said first nuclease is SEQ ID NO: 137 and said second nuclease is SEQ ID NO: 138.

13. The method of claim 1, wherein the genes encoding said first and second nucleases are delivered to the cell using a recombinant adeno-associated virus (AAV).

14. The method of claim 1, wherein said first and second recognition sites are selected to have identical overhangs if cut by the first and second nucleases.

15. A method for removing a DNA sequence from the genome of a cell, the method comprising contacting the DNA with a first nuclease that cuts a first recognition site and a second nuclease that cuts a second recognition site, wherein said first and second recognition sites are selected to have identical overhangs if cut by the first and second nucleases.

16. The method of claim 15 wherein, the genes encoding said first and second nucleases are delivered to the cell using a recombinant adeno-associated virus (AAV).

17. The method of claim 15, wherein said first and second nucleases are meganucleases.

18. The method of claim 15, wherein said first and second nucleases are CRISPRs.

19. The method of claim 15, wherein said first and second nucleases are compact TALENs.
